(19) United States
(12) Patent Application Publication
(10) Pub. No.: US 2022/0267444 A1
(43) Pub. Date: Aug. 25, 2022

(54) ANTI-PD-1 ANTIBODY AND MEDICAL USE THEREOF

(71) Applicant: CTTQ-AKESO (SHANGHAI) BIOMED. TECH. CO., LTD., Shanghai (CN)

(72) Inventors: Zhongmin WANG, Zhongshan, Guangdong (CN); Peng ZHANG, Zhongshan, Guangdong (CN); Baiyong LI, Zhongshan, Guangdong (CN); Yu XIA, Zhongshan, Guangdong (CN)

(21) Appl. No.: 17/630,477

(22) PCT Filed: Jul. 31, 2020
(86) PCT No.: PCT/CN2020/106219

§ 371 Date: Jan. 26, 2022

(30) Foreign Application Priority Data
Aug. 2, 2019 (CN) 201910711138.5
Nov. 13, 2019 (CN) 201911105711.4
Nov. 13, 2019 (CN) 201911105715.2
Nov. 19, 2019 (CN) 201911133858.4

(51) Int. Cl. C07K 16/28; G01N 33/574; A61K 31/47; A61K 31/475; A61K 39/395; A61P 35/00

(52) U.S. Cl. CPC C07K 16/2818; G01N 33/57492; A61K 31/47; A61K 31/475; A61K 39/3955; A61K 2039/545; C07K 2317/71; C07K 2317/52; C07K 2317/92; C07K 2317/622; A61P 35/00

(57) ABSTRACT

The present invention relates to the field of tumor treatment and molecular immunology, and particularly, to an anti-PD-1 antibody and pharmaceutical use thereof. Specifically, the present invention relates to a monoclonal antibody, wherein a heavy chain variable region of the monoclonal antibody comprises CDRs with amino acid sequences of SEQ ID NOs: 19-21, and/or a light chain variable region of the monoclonal antibody comprises CDRs with amino acid sequences of SEQ ID NOs: 22-24; wherein according to the EU numbering system, a heavy chain constant region of the antibody comprises mutations at any 2 or 3 of positions 234, 235 and 237, and an affinity constant of the antibody to FcγRIIIa and/or C1q is reduced after the mutation as compared to that before the mutation. The monoclonal antibody of the present invention can be well and specifically bind to PD-1, specifically relieve immunosuppression of PD-1 in an organism and activate T lymphocytes.

### TECHNICAL FIELD

The present invention relates to the field of tumor treatment and molecular immunology, and particularly, to an anti-PD-1 antibody and pharmaceutical use thereof. More particularly, the present invention relates to mutant anti-PD-1 antibodies.

### BACKGROUND

The transmembrane receptor PD-1 (programmed cell death protein 1) is a member of the CD28 family, and is expressed in activated T cells, B cells and myeloid cells. Both ligands of PD-1, PDL1 (programmed cell death 1 ligand 1, or PDL-1) and PDL2 (programmed cell death 1 ligand 2, or PDL-2), are members of the B7 superfamily. PDL1 is expressed in a variety of cells including T cells, B cells, endothelial cells and epithelial cells, and PDL2 is expressed only in antigen presenting cells such as dendritic cells and macrophages.

The PD-1/PDL1 signaling pathway plays an important role in regulating immune tolerance, microbial infection and tumor immune escape. PD-1 is mainly expressed in immune cells such as T cells, and the ligand PDL1 of PD-1 is highly expressed in a plurality of human tumor tissues. Blocking the PD-1/PDL1 signaling pathway may activate inhibited T cells, which thus attack cancer cells. Blocking the PD-1/PDL1 signaling can promote the proliferation of tumor antigen-specific T cells, activate tumor cell killing process and further inhibit local tumor growth (Julie R et al., 2012, N Engl J Med., 366:2455-2465). PD-1/PD-L1 is an important specific immune checkpoint. The formation of a PD-1/PD-L1 complex transmits inhibitory signals and negatively regulates the immune response of T cells. It inhibits TCR-mediated T cell activation, cytokine production and T cell proliferation (Fife et al., (2011) Nature Immunology 10:1185-1193), induces the depletion or anergy in homologous antigen-specific T cells (Hofmeyer et al., (2011) Journal of Biomedicine and Biotechnology, 2011:1-9), promotes the differentiation of Th1 cells into Foxp3+ regulatory T cells (Armanath et al., (2011) Science Trans. Med., 3:1-13; Francisco et al., (2009) J. Exp. Med., 206:3015-3029), and induces the apoptosis of effector T cells. The disruption of PD-L1 genes results in an upregulated T cell response and the production of autoreactive T cells (Latchman et al., (2004) PNAS, 101:10691-10696). The blockade of PD-1 or PD-L1 by antibody leads to elevated anti-tumor immunity (Iwai et al., (2002) PNAS, 99:12293-12297). In the past nearly 20 years, researchers have made great efforts to develop a specific immune checkpoint inhibitor, expecting to provide new immunotherapeutic regimens for treating cancer. Among these, the innate T-lymphocyte immune system can respond to a variety of tumor antigens owning to its high anti-cancer capacity and broad and precise specificity. This emerging cancer immunotherapy enhances the anti-tumor immune response by the adoptive transfer of activated effector cells, the immunization against relevant antigens, or the provision of non-specific immunostimulants. Thus, PD-1/PD-L1-specific immune checkpoint inhibitors have potential for treating related cancers.

The mechanism of action of anti-PD-1 antibodies is to block the binding of PD-1 proteins on the surfaces of immune cells to ligands PDL1 or PDL2 thereof, and to activate the immune cells to kill a tumor. At present, there is still a need for developing a novel anti-PD-1 antibody to reduce or eliminate the damage caused by antibody-mediated ADCC, ADCP and/or CDC activity on immune cells to which the anti-PD-1 antibody binds, and to improve the efficacy of the antibody therapy. ADCC (antibody-dependent cell-mediated cytotoxicity) refers to killing of a target cell by a killer cell (NK cell, macrophage, etc.) that is mediated by binding of the Fab fragment of an antibody to an epitope of a virus-infected cell or a tumor cell and binding of the Fc fragment of the antibody to an Fc receptor (FcR) on the surface of the killer cell.

CDC (Complement-dependent cytotoxicity) refers to a lytic effect on target cells by a membrane-attacking complex that is formed by serial bindings of an antibody to corresponding antigens on the surfaces of the cell membranes and the complement C1q and activation of C2-C9.

Fc receptors belong to an immunoglobulin family that are expressed on the surface of specific immune cells to recognize antibody Fc regions and mediate immune responses. After the Fab region recognizes an antigen, the Fc region of the antibody binds to the Fc receptor on the immune cell (e.g., a killer cell) to initiate the response function of the immune cell, such as phagocytosis and ADCC.

According to the type of antibody recognized by the Fc receptor and the type of expression cells, Fc receptors are mainly classified into three types, FcγR, FcαR and FcεR. FcγR can be further classified into four subtypes, FcγRI (CD64), FcγRII (CD32), FcγRIII (CD16) and FcRn (neonatal Fc receptor). Among these, FcγRI, FcγRII and FcγRIII are closely associated with ADCC effect. FcγRIII is the most predominant molecule mediating ADCC, with two highly homologous subtypes, FcγRIIIa and FcγRIIIb, in different cell types. In FcγRIIIa populations, two subtypes distinguished by sites of single-nucleotide polymorphism (SNP), FcγRIIIa_V158 with high affinity and FcγRIIIa_F158 with low affinity, are present. FcγRI has higher affinity for the Fc region of IgG and participates in ADCC process; FcγRII comprises three subtypes, FcγRIIa, FcγRIIb and FcγRIIc (also referred to as CD32a, CD32b and CD32c, respectively), among which FcγRIIa has ADCC activity; for FcγRIIa, two subtypes, FcγRIIa_H131 and FcγRIIa_R131, are present in humans due to single nucleotide mutation; FcγRIIb is an inhibitory receptor, and is a typical inhibitory FcγR that inhibits nearby ITAM pathways. For example, after the binding of the immune complex to BCR, the Fc fragment binds to FcγRIIb on the same cell, negatively regulating B cell activation and decreasing secretion of antibodies and cytokines (Hogarth P M, Pietersz G A., 2012, NATURE REVIEWS DRUG DISCOVERY, 11(4):311-331).

The IgG family comprises four members, IgG1, IgG2, IgG3 and IgG4, which differ in amino acids in the fragment crystallizable (Fc) region of the heavy chain constant region, resulting in their varying affinities for FcγRs. IgG1 is the most abundant subtype in humans and is also the most common subtype used in monoclonal antibody medication. IgG1 is capable of binding various FcγRs and is able to induce ADCC and CDC effects. IgG2 has the lowest affinity for FcγRs, but is still able to induce monocyte-mediated ADCC by binding to FcγRIIa. IgG3 features the highest binding capacity to FcγRs, and can induce ADCC and a greater CDC effect than IgG1. IgG4 molecules demonstrate a weak binding to FcγRs other than FcγRI, having a lower probability of causing CDC and NK cell-mediated ADCC. However, antibodies of the IgG4 subtype can mediate ADCP effects through binding to FcγRI, and the ADCP effects, present in antibody therapies targeting immune cells, may cause damage to immune cells, posing pharmacological adverse effects.

Zhang et al. (Zhang T et al, Cancer Immunol Immunother., 2018; 67(7):1079-1090) and Dahan et al. (Dahan R et al., Cancer cell, 2015, 28(3):285-95) reported that the binding of Fc fragments of antibodies targeting immune checkpoints such as PD-1 and CTLA-4 to Fc receptors negatively affects antibody-mediated anti-cancer activity, possibly due to Fc-dependent effector function-induced immune cell damage including antibody-dependent cell-mediated cytotoxicity, where antibody-dependent cellular phagocytosis (ADCP) is an important mechanism leading to immune cell damage.

Non-squamous non-small cell lung cancer (NSCLC) and squamous non-small cell lung cancer (sNSCLC) are both lung tissue malignancies. Current therapeutic strategies include early stage surgery. However, most diagnosed lung cancer patients are at the advanced stage, showing poor response to surgery and radiotherapy. Thus chemotherapy has become an important treatment. Currently, combined chemotherapy of platinum and other chemotherapeutics is still the first-line chemotherapy for lung cancer including advanced sNSCLC and NSCLC (Pfister D G. et al., J. Clin. Oncol., 2003, 22:330; De Ruysscher et al., (2006) Annals of Oncology, 17:543-552).

Chemotherapies are currently mainly classified into the following nine classes (He Jie, et al., Clinical Oncology, Beijing, People's Medical Publishing House, 2016:230-237). The first class are drugs that directly bind to DNA and prevent DNA replication, including various alkylating agents, mitomycin, bleomycin, dacarbazine, platinum-based drugs (e.g., cisplatin and carboplatin), camptothecins, and derivatives thereof. The second class are drugs for preventing nucleic acid biosynthesis, which mainly affect the enzyme system of tumor cells and block the synthesis of precursors of DNA and RNA, thereby inhibiting the formation of DNA or RNA, including methotrexate, fluorouracil, 6-mercaptopurine, hydroxyurea and cytarabine; such drugs mainly act on cells in S phase, and are antimetabolite chemotherapeutic drugs and cell cycle-specific anticancer drugs. The third class are chemotherapeutic drugs which affect transcription through the pharmacological mechanism that the drugs are inserted into the DNA double helix to form non-covalent binding with the DNA double helix, interfering with the transcription of genetic information on DNA to the DNA-dependent mRNA and causing compromised template function and hindered transcription. The fourth class are those affecting tubulin and mitosis, including vinca alkaloids, podophyllotoxins and taxanes. The fifth class are drugs affecting the function of ribosomes and blocking protein synthesis; representatives of such drugs are harringtonines, which inhibit the initiation of protein synthesis, decompose the ribosome and release new peptide chain, but do not block the binding of mRNA and tRNA to ribosomes; such drugs cause the reduction of nuclear DNA and cytoplasmic RNA and depolymerization of polysomes, and inhibit mitosis. The sixth class are drugs that affect the tumor cell membrane such as concanavalin (Con-A) and phytohemagglutinin (PHA); they can bind to glycoprotein receptors on the cell membrane, thereby affecting DNA synthesis in tumor cells and preventing tumor cell from dividing. The seventh class are drugs that induce apoptosis, such as arsenic trioxide. The eighth class are hormones that treat tumors by regulating the endocrine system, including estrogens, antiestrogens, progestogens, androgens, antiandrogens, corticosteroids, and anticorticosteroids (including dichlorodiphenyldichloroethane and aminoglutethimide). The ninth class are anticancer targeted therapies, including monoclonal antibodies, epidermal growth factor signaling inhibitors (e.g., targeted drugs against receptor tyrosine kinase pathway), ubiquitin-proteasome inhibitors, and angiogenesis inhibitors. However, in addition to killing tumor cells, chemotherapeutics also damage normal human cells, so conventional chemotherapy regimens for cancer patients often cause serious toxic and side effects. More importantly, in addition to obvious toxicity, chemotherapeutics only demonstrate a short-term control over the diseases and a low 5-year survival rate in patients receiving chemotherapeutics. Therefore, developing a medication or combination therapy with lower toxicity and higher efficacy is of great meaning.

Anlotinib is a quinoline derivative tyrosine kinase inhibitor. As a multi-target tyrosine kinase inhibitor (TKI), it affects tumor angiogenesis and proliferation signal transduction. The major targets include: receptor tyrosine kinases vascular endothelial growth factor receptors (VEGFRs) 1 to 3, epidermal growth factor receptor (EGFR), fibroblast growth factor receptors (FGFRs) 1 to 4, platelet-derived growth factor receptors (PDGFRs) a and 13, and stem cell factor receptors (SCFRs) 7, 8 and 9. A phase 2 trial showed that anlotinib improved progression-free survival with the potential benefit for overall survival (Han B, et al., Br J Cancer, 2018; 118(5):654-661). A multicenter, double-blind, randomized phase 3 clinical trial showed that anlotinib resulted in extended overall and progression-free survivals in Chinese patients. The finding suggested that anlotinib is well tolerated and is a potential third-line or further treatment for patients with advanced NSCLC (Han B, et al., JAMA Oncol., 2018 Nov.; 4(11):1569-1575).

Example 24 of Patent No. WO2008112407 discloses a quinoline-derived tyrosine kinase inhibitor 1-[[[4-(4-fluoro-2-methyl-1H-indol-5-yl)oxy-6-methoxyquinolin-7-yl]oxy]methyl]cyclopropylamine and a method for preparing the same. The structural formula of the quinoline-derived tyrosine kinase inhibitor is shown in formula I. Anlotinib hydrochloride is the hydrochloride salt of the compound of formula I.

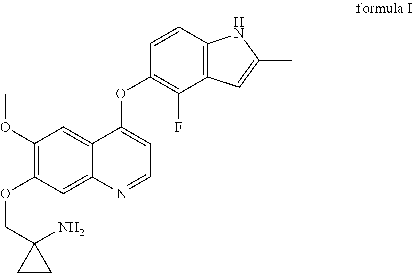 [SMILES: COc1cc2c(Oc3ccc4[nH]c(C)cc4c3F)ccnc2cc1OCC1(N)CC1]

Lenvatinib, an oral multiple tyrosine kinase inhibitor developed by Eisai (Japan), is a multi-target receptor tyrosine kinase inhibitor that inhibits the kinase activity of VEGFR1 (FLT1), VEGFR2 (KDR) and VEGFR3 (FLT4). In addition to normal cellular function, lenvatinib also inhibits other receptor tyrosine kinases involved in pathogenic angiogenesis, tumor growth and cancer progression, including fibroblast growth factor (FGF) receptors FGFR1, FGFR2, FGFR3 and FGFR4, “rearranged during transfection” (RET) receptor, KIT and platelet-derived growth factor receptor α (PDGFRα). Lenvatinib also exhibits antiproliferative activity in hepatocellular carcinoma cell lines, which is dependent on activated FGFR signaling and simultaneous inhibition of phosphorylation of FGF receptor substrate 2α (FRS2α).

The structure of lenvatinib, 4-(3-chloro-4(cyclopropylaminocarbonyl)aminophenoxy)-7-methoxy-6-quinolinecarboxamide, is disclosed in Example 368 of U.S. Pat. No. 7,612,208. U.S. Pat. No. 7,253,286 discloses the mesylate salt form of lenvatinib (i.e., lenvatinib mesylate), named 4-[3-chloro-4-(cyclopropylureido)phenoxy]-7-methoxyquinoline-6-carboxamide mesylate, the chemical structure of which is provided below (formula II):

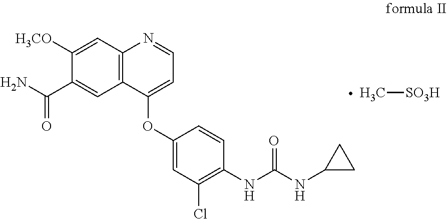

However, for a variety of tumors, the disease is still uncontrollable for a long term after chemotherapy, and the 5-year survival rate is still very low. Therefore, developing a medication or combination therapy with lower toxicity and higher efficacy is of great meaning.

### SUMMARY

By intensive research and creative efforts, the inventor correspondingly modified the Fc fragment of the anti-PD-1 antibody structure to reduce the binding capacity of the Fc region to Fc receptors, thereby reducing ADCC, ADCP and/or CDC effects on T cells and increasing the efficacy of the anti-PD-1 antibody. The present invention is detailed below.

One aspect of the present invention relates to an antibody, wherein a heavy chain variable region of the antibody comprises HCDR1-HCDR3 with amino acid sequences set forth in SEQ ID NOs: 19-21, respectively, and a light chain variable region of the antibody comprises LCDR1-LCDR3 with amino acid sequences set forth in SEQ ID NOs: 22-24, respectively;

the antibody is of human IgG1 subtype;

wherein, according to the EU numbering system, a heavy chain constant region of the antibody comprises mutations at any 2 or 3 of positions 234,235 and 237, and an affinity constant of the antibody to FcγRIIIa and/or C1q is reduced after the mutation as compared to that before the mutation; preferably, the affinity constant is measured by a Fortebio Octet system.

In one embodiment of the present invention, the antibody is a monoclonal antibody. In one embodiment of the present invention, the antibody is an anti-PD-1 antibody, preferably an anti-PD-1 monoclonal antibody.

In some embodiments of the present invention, for the antibody, according to the EU numbering system, the heavy chain constant region of the antibody comprises the following mutations at positions 234,235 and/or 237:

L234A and L235A;

L234A and G237A;

L235A and G237A;

or

L234A, L235A and G237A.

In the present invention, letters before the position number represent amino acids before mutation, and letters after the position number represent amino acids after mutation, unless otherwise specified.

The present invention also relates to an antibody, wherein a heavy chain variable region of the antibody comprises HCDR1-HCDR3 with amino acid sequences set forth in SEQ ID NOs: 19-21, respectively, and a light chain variable region of the antibody comprises LCDR1-LCDR3 with amino acid sequences set forth in SEQ ID NOs: 22-24, respectively;

the antibody is of human IgG1 subtype;

wherein according to the EU numbering system, a heavy chain constant region of the antibody comprises the following mutations at positions 234,235 and/or 237:

L234A and L235A;

L234A and G237A;

L235A and G237A;

or

L234A, L235A and G237A.

In some embodiments of the present invention, according to the EU numbering system, the heavy chain constant region of the antibody further comprises one or more mutations selected from:

N297A, D265A, D270A, P238D, L328E, E233D, I1268D, P271G, A330R, C226S, C229S, E233P, P331S, S267E, L328F, A330L, M252Y, S254T, T256E, N297Q, P238S, P238A, A327Q, A327G, P329A, K322A, T394D, G236R, G236A, L328R, A330S, P331S, H268A, E318A and K320A.

In some embodiments of the present invention, for the antibody,

the heavy chain variable region of the antibody comprises an amino acid sequence selected from SEQ ID NO: 2 and SEQ ID NO: 6; and

the light chain variable region of the antibody comprises an amino acid sequence selected from SEQ ID NO: 4 and SEQ ID NO: 8.

In some embodiments of the present invention, for the antibody, the heavy chain variable region of the antibody comprises an amino acid sequence set forth in SEQ ID NO: 2, and the light chain variable region of the antibody comprises an amino acid sequence set forth in SEQ ID NO: 4;

the heavy chain variable region of the antibody comprises an amino acid sequence set forth in SEQ ID NO: 2, and the light chain variable region of the antibody comprises an amino acid sequence set forth in SEQ ID NO: 8;

the heavy chain variable region of the antibody comprises an amino acid sequence set forth in SEQ ID NO: 6, and the light chain variable region of the antibody comprises an amino acid sequence set forth in SEQ ID NO: 4; or

the heavy chain variable region of the antibody comprises an amino acid sequence set forth in SEQ ID NO: 6, and the light chain variable region of the antibody comprises an amino acid sequence set forth in SEQ ID NO: 8.

In one embodiment of the present invention, for the antibody,

the heavy chain is set forth in SEQ ID NO: 16, and the light chain is set forth in SEQ ID NO: 12;

or

the heavy chain is set forth in SEQ ID NO: 18, and the light chain is set forth in SEQ ID NO: 12.

The variable regions of the light chain and the heavy chain determine the binding of the antigen; the variable region of each chain comprises three hypervariable regions, i.e., complementarity determining regions (CDRs) (the CDRs of the heavy chain (H) include HCDR1, HCDR2, HCDR3, and the CDRs of the light chain (L) include LCDR1, LCDR2, LCDR3; defined by Kabat et al., see Sequences of Proteins of Immunological Interest, Fifth Edition (1991), Volumes 1-3, NIH Publication 91-3242, Bethesda Md.).

The amino acid sequences of the CDR regions of the monoclonal antibody in (1) to (3) above are analyzed by technical means well known to those skilled in the art, for example, by a VBASE2 database:

The antibodies 14C12, 14C12H1L1(hG1WT), 14C12H1L1(hG1DM) and 14C12H1L1(hG1TM) involved in the present invention have the same CDRs.

The amino acid sequences of the 3 CDR regions of the heavy chain variable region are as follows:

HCDR1:
(SEQ ID NO: 19)
GFAFSSYD,
HCDR2:
(SEQ ID NO: 20)
ISGGGRYT,
and
HCDR3:
(SEQ ID NO: 21)
ANRYGEAWFAY.

The amino acid sequences of the 3 CDR regions of the light chain variable region are as follows:

LCDR1:
(SEQ ID NO: 22)
QDINTY,
LCDR2:
(SEQ ID NO: 23)
RAN,
and
LCDR3:
(SEQ ID NO: 24)
LQYDEFPLT.

In some embodiments of the present invention, the antibody binds to FcγRIIIa_F158, FcγRI, FcγRIIa_H131, FcγRIIIa_V158 and/or FcγRIIb with an affinity constant greater than about 10⁻⁷ M, for example, greater than about 10⁻⁶ M, 10⁻⁵ M, 10⁻⁴ M, or 10⁻³ M or greater;

preferably, the affinity constant is measured by a Fortebio Octet system;

preferably, the antibody has no binding signal or a binding signal of less than 0.1 nm to

FcγRIIIa_F158, FcγRI, FcγRIIa_H131, FcγRIIIay158 and/or FcγRIIb; preferably, the binding signal refers to a response measured by a Fortebio Octet system.

In some embodiments of the present invention, the antibody binds to C1q with an affinity constant greater than about 10⁻⁹ M, for example, greater than about 10⁻⁸ M, 10⁻⁷ M, 10⁻⁶ M, or 10⁻⁵ M or greater; preferably, the affinity constant is measured by a Fortebio Octet system;
preferably, the antibody has no binding signal or a binding signal of less than 0.1 nm to C1q;

preferably, the binding signal refers to a response measured by a Fortebio Octet system.

In some embodiments of the present invention, the antibody is a monoclonal antibody.

In some embodiments of the present invention, the antibody is a humanized antibody.

Another aspect of the present invention relates to an isolated nucleic acid molecule encoding the antibody according to any embodiment of the present invention.

Yet another aspect of the present invention relates to a vector comprising the isolated nucleic acid molecule disclosed herein.

Yet another aspect of the present invention relates to a host cell comprising the isolated nucleic acid molecule or the vector disclosed herein.

Yet another aspect of the present invention relates to a conjugate, comprising an antibody and a conjugated moiety, wherein the antibody is the antibody according to any embodiment of the present invention, and the conjugated moiety is a detectable label; preferably, the conjugated moiety is a radioisotope, a fluorescent substance, a luminescent substance, a colored substance, or an enzyme.

Yet another aspect of the present invention relates to a kit comprising the antibody according to any embodiment of the present invention or comprising the conjugate disclosed herein; preferably, the kit further comprises a second antibody specifically recognizing the antibody; optionally, the second antibody further comprises a detectable label, for example, a radioisotope, a fluorescent substance, a luminescent substance, a colored substance, or an enzyme.

Yet another aspect of the present invention relates to use of the antibody or the conjugate according to any embodiment of the present invention in preparing a kit for detecting the presence or level of PD-1 in a sample.

Yet another aspect of the present invention relates to a pharmaceutical composition comprising the antibody or the conjugate according to any embodiment of the present invention; optionally, the pharmaceutical composition further comprises a pharmaceutically acceptable carrier and/or excipient.

In one or more embodiments of the present invention, the pharmaceutical composition further comprises one or more anti-tumor chemotherapeutics;

preferably, the anti-tumor chemotherapeutic is a tyrosine kinase inhibitor; more preferably, the anti-tumor chemotherapeutic is anlotinib or a pharmaceutically acceptable salt thereof (e.g., hydrochloride salt), or lenvatinib or a pharmaceutically acceptable salt thereof (e.g., mesylate salt).

In one or more embodiments of the present invention, the unit dose of the pharmaceutical composition is 100-1000 mg, 200-800 mg, 200-500 mg, 300-600 mg, 400-500 mg, or 450 mg, based on the mass of the antibody.

Yet another aspect of the present invention relates to a therapeutic combination comprising:

the antibody according to any embodiment of the present invention, and at least one (e.g., 1, 2 or 3) anti-tumor chemotherapeutic.

In one or more embodiments of the present invention, for the therapeutic combination, the anti-tumor chemotherapeutic is a tyrosine kinase inhibitor; preferably, the anti-tumor chemotherapeutic is anlotinib or a pharmaceutically acceptable salt thereof (e.g., hydrochloride salt), or lenvatinib or a pharmaceutically acceptable salt thereof (e.g., mesylate salt).

In one or more embodiments of the present invention, for the therapeutic combination, the unit dose of the antibody is 100-1000 mg, 200-800 mg, 200-500 mg, 300-600 mg, 400-500 mg, or 450 mg.

In one or more embodiments of the present invention, for the therapeutic combination, the unit dose of the anti-tumor chemotherapeutic is 0.1-100 mg, 0.5-50 mg, 0.5-10 mg, 1-10 mg, 2-8 mg, or 1-5 mg.

In one or more embodiments of the present invention, for the therapeutic combination, the unit dose of the anti-tumor chemotherapeutic is 1-20 mg, 2-15 mg, 4-12 mg, or 8-12 mg.

In one or more embodiments of the present invention, for the therapeutic combination, wherein

the therapeutic combination is a fixed combination, e.g., in the form of a solid pharmaceutical composition or a liquid pharmaceutical composition; or

the therapeutic combination is a non-fixed combination, e.g., the anti-PD-1 antibody and the anti-tumor chemotherapeutic in the non-fixed combination are each in the form of a pharmaceutical composition.

Yet another aspect of the present invention relates to a kit product comprising the pharmaceutical composition according to any embodiment of the present invention or the therapeutic combination according to any embodiment of the present invention, and a package insert.

Yet another aspect of the present invention relates to use of the antibody according to any embodiment of the present invention, the conjugate disclosed herein, the pharmaceutical composition according to any embodiment of the present invention or the therapeutic combination according to any embodiment of the present invention in preparing a medicament for treating and/or preventing a tumor or anemia, or in preparing a medicament for diagnosing a tumor or anemia, wherein preferably the tumor is selected from one or more of melanoma, renal cancer, prostate cancer, bladder cancer, colon cancer, rectal cancer, gastric cancer, liver cancer, lung cancer, ovarian cancer, leukemia, nasopharyngeal cancer and endometrial cancer;

preferably, the lung cancer is selected from one or more of non-small cell lung cancer, small cell lung cancer and squamous cell lung cancer;

preferably, the gastric cancer is gastric adenocarcinoma or gastroesophageal junction adenocarcinoma;

preferably, the tumor is a solid tumor of MSI-H/dMMR phenotype; preferably, the tumor is selected from one or more of the following tumors of MSI-H/dMMR phenotype: colon cancer, rectal cancer, endometrial cancer, gastric cancer, mesothelioma, sarcoma, adrenocortical carcinoma, malignant melanoma and ovarian germ cell neoplasm.

In one or more embodiments of the present invention, for the use, the tumor is a recurrent, metastatic (e.g., lymphatic metastasis, brain metastasis, and/or bone metastasis) or refractory tumor.

MSI refers to microsatellite instability. Microsatellites are short tandem repeats throughout the human genome, including 10-50 repeats of one, two or more nucleotides. Microsatellites in certain abnormal cells, such as tumors, are altered in length by insertion or deletion of repeat units as compared to normal cells. Such alteration is referred to as MSI. Based on instability and extent, MSI can be classified as microsatellite instability-high (MSI-H), microsatellite instability-low (MSI-L) and microsatellite stable (MSS). The major cause of MSI is DNA mismatch repair (MMR) deficiency. Human mismatch repair genes (MMR genes) can express corresponding mismatch repair proteins through transcription and translation. Absence of any MMR protein may lead to mismatch repair deficiency, and basepair mismatch will accumulate in the process of DNA replication due to such deficiency, ultimately resulting in MSI. About 15% of colorectal cancers are attributed to the MSI pathway. This was first reported in colorectal cancer, and may also occur in gastric cancer, endometrial cancer, adrenocortical carcinoma and the like (Baretti M et al., Pharmacol Ther., 2018; 189:45-62). MSI-H/dMMR characteristics were also found in mesothelioma, sarcoma, adrenocortical carcinoma, malignant melanoma and ovarian germ cell neoplasm in subsequent studies.

MSI-H and dMMR represent the results of two different assays and are biologically consistent, called MSI-H/dMMR or MSI-high/dMMR, while MSI-L and MSS are phenotypes of proficient MMR (pMMR). The detection of dMMR is to perform an immunohistochemical assay of protein expression for four mismatch genes of MSH2, MLH1, MSH6 and PMS2 based on tumor specimens (including surgical specimens and aspiration specimens). Absence of any of the four proteins confirms the dMMR; positive results of all the four proteins indicate pMMR, i.e., a complete mismatch repair function. The detection of MSI is to match the length of the repeated DNA sequences (microsatellite sequences) in tumor cells and somatic cells, and to compare the lengths. When 5 standard loci are detected using PCR based on the American NCI standard, inconsistencies in two or more loci indicate instability, defined as MSI-H, one inconsistent locus indicates MSI-L, and 5 consistent loci indicate MSS. High-throughput sequencing (also referred to as next-generation sequencing, or NGS) can also be used as a method for detecting microsatellite instability. When more microsatellite loci are selected, such as more than 5 loci or additional microsatellite loci, for PCR assay, inconsistency in >30% loci is defined as MSI-H, consistency in all loci is defined as MSS, and inconsistency between 0 and 30% is defined as MSI-L.

Yet another aspect of the present invention relates to use of the antibody according to any embodiment of the present invention, the conjugate described herein, the pharmaceutical composition according to any embodiment of the present invention or the therapeutic combination according to any embodiment of the present invention in preparing:

a medicament for blocking the binding of PD-1 to PD-L1,

a medicament for down-regulating the activity or level of PD-1,

a medicament for relieving the immunosuppression of PD-1 in an organism, or

a medicament for elevating IFN-γ and/or IL-2 expression in T lymphocytes.

Interferon γ (IFN-γ) is produced primarily and innately by natural killer cells (NK) and natural killer T cells (NKT) and is produced by effector T cells such as CD4 Th1 cells and CD8 cytotoxic T lymphocytes stimulated by specific antigens. As an important innate and acquired immune cytokine, IFN-γ plays an important role in fighting or inhibiting viral infection and certain bacterial and protozoal disease infections. Meanwhile, IFN-γ can activate macrophages, induce the expression of type II major histocompatibility complex, and activate the immune response to control tumor progression (Schoenborn J R, Wilson C B., Regulation of Interferon-γ During Innate and Adaptive Immune Responses, Advances in Immunology, 2007, 96:41-101). In the in vitro experiment of the present invention, the antibody disclosed herein can induce the IFN-γ secretion to activate the immune response. Interleukin 2 (IL-2) is produced by T cells. It is a growth factor that regulates T cell subgroups, and an important factor in regulating immune responses. It promotes the proliferation of activated B cells, and participates in antibody responses, hematopoiesis and tumor surveillance. Recombinant human IL-2 has been approved by the U.S. FDA for treating malignancies, including melanoma and renal tumor (Chavez, A. R., et al., Pharmacologic administration of interleukin-2, Ann. N.Y. Acad. Sci., 2009, 1182:p. 14-27). In-vitro studies demonstrated that the antibody disclosed herein can specifically relieve the immunosuppression of PD-1, activate T cells, and induce IL-2 generation, and is promising in wide applications in therapies against diseases such as tumors and parasite infections.

Yet another aspect of the present invention relates to an in vivo or in vitro method comprising: administering to a subject in need an effective amount of the antibody according to any embodiment of the present invention, the conjugate described herein, the pharmaceutical composition according to any embodiment of the present invention or the therapeutic combination according to any embodiment of the present invention. The method is selected from:

a method for blocking the binding of PD-1 to PD-L1,

a method for down-regulating the activity or level of PD-1,

a method for relieving the immunosuppression of PD-1 in an organism, or

a method for elevating IFN-γ and/or IL-2 expression in T lymphocytes.

Also related are the antibody according to any embodiment of the present invention, the conjugate described herein, the pharmaceutical composition according to any embodiment of the present invention or the therapeutic combination according to any embodiment of the present invention for use in treating and/or preventing a tumor or anemia, or in diagnosing a tumor or anemia, wherein preferably the tumor is selected from one or more of melanoma, renal cancer, prostate cancer, bladder cancer, colon cancer, rectal cancer, gastric cancer, liver cancer, lung cancer, ovarian cancer, leukemia, nasopharyngeal cancer and endometrial cancer;

preferably, the lung cancer is selected from one or more of non-small cell lung cancer, small cell lung cancer and squamous cell lung cancer;

preferably, the gastric cancer is gastric adenocarcinoma or gastroesophageal junction adenocarcinoma;

preferably, the tumor is a solid tumor of MSI-H/dMMR phenotype; preferably, the tumor is selected from one or more of the following tumors of MSI-H/dMMR phenotype: colon cancer, rectal cancer, endometrial cancer, gastric cancer, mesothelioma, sarcoma, adrenocortical carcinoma, malignant melanoma and ovarian germ cell neoplasm. In one or more embodiments of the present invention, for the antibody or the conjugate described herein, the tumor is a recurrent, metastatic (e.g., lymphatic metastasis, brain metastasis, and/or bone metastasis) or refractory tumor.

The antibody according to any embodiment of the present invention, the conjugate described herein, the pharmaceutical composition according to any embodiment of the present invention or the therapeutic combination according to any embodiment of the present invention are used for:

blocking the binding of PD-1 to PD-L1,

down-regulating the activity or level of PD-1,

relieving the immunosuppression of PD-1 in an organism, or

elevating IFN-γ and/or IL-2 expression in T lymphocytes.

Yet another aspect of the present invention relates to a method of treating and/or preventing a tumor or anemia, or a method of diagnosing a tumor or anemia, comprising: administering to a subject in need an effective amount of the antibody according to any embodiment of the present invention, the conjugate described herein, the pharmaceutical composition according to any embodiment of the present invention or the therapeutic combination according to any embodiment of the present invention, wherein preferably the tumor is selected from one or more of melanoma, renal cancer, prostate cancer, bladder cancer, colon cancer, rectal cancer, gastric cancer, liver cancer, lung cancer, ovarian cancer, leukemia, nasopharyngeal cancer and endometrial cancer;

preferably, the lung cancer is selected from one or more of non-small cell lung cancer, small cell lung cancer and squamous cell lung cancer;

preferably, the gastric cancer is gastric adenocarcinoma or gastroesophageal junction adenocarcinoma;

preferably, the tumor is a solid tumor of MSI-H/dMMR phenotype; preferably, the tumor is selected from one or more of the following tumors of MSI-H/dMMR phenotype: colon cancer, rectal cancer, endometrial cancer, gastric cancer, mesothelioma, sarcoma, adrenocortical carcinoma, malignant melanoma and ovarian germ cell neoplasm.

In one or more embodiments of the present invention, for the method, the tumor is a recurrent, metastatic (e.g., lymphatic metastasis, brain metastasis, and/or bone metastasis) or refractory tumor.

In one or more embodiments of the present invention, for the method, the administration is before or after a surgical treatment and/or before or after a radiotherapy.

In one or more embodiments of the present invention, the method, wherein the unit dose of the anti-PD-1 antibody is 0.1-100 mg, preferably 1-10 mg (e.g., 1 mg, 2 mg, 3 mg, 4 mg, 5 mg, 6 mg, 7 mg, 8 mg, 9 mg or 10 mg) per kg body weight; alternatively, the unit dose of the anti-PD-1 antibody is 10-1000 mg (e.g., about 100 mg, about 150 mg, about 200 mg, about 250 mg, about 300 mg, about 350 mg, about 400 mg, about 450 mg, about 500 mg, about 600 mg, about 700 mg, about 800 mg, about 900 mg or about 1000 mg), preferably 50-500 mg, 100-400 mg, 150-300 mg, 150-250 mg or 200 mg in each subject;

preferably, the dose is given once every 3 days, 4 days, 5 days, 6 days, 10 days, 1 week, 2 weeks or 3 weeks;

preferably, the route of administration is intravenous drip infusion or intravenous injection. In some embodiments, the administration of the anti-PD-1 antibody is performed in cycles of 2 weeks (14 days) or 3 weeks (21 days), and preferably, the anti-PD-1 antibody is administered intravenously on the first day (D1) of each cycle. For example, the anti-PD-1 antibody is administered once every two weeks (q2w) or three weeks (q3w).

In the present invention, unless otherwise defined, the scientific and technical terms used herein have the meanings generally understood by those skilled in the art. In addition, the laboratory operations of cell culture, molecular genetics, nucleic acid chemistry and immunology used herein are the routine procedures widely used in the corresponding fields.

Meanwhile, in order to better understand the present invention, the definitions and explanations of the relevant terms are provided below.

As used herein, when referring to the amino acid sequence of PD-1 protein (programmed cell death protein 1, NCBI GenBank: NP_005009.2), it includes the full length of the PD-1 protein, or the extracellular fragment PD-1ECD of PD-1 or a fragment comprising PD-1ECD, and it also includes a fusion protein of PD-1ECD, such as a fragment fused to an Fc protein fragment of a mouse or human IgG (mFc or hFc). However, those skilled in the art will appreciate that in the amino acid sequence of the PD-1 protein, mutations or variations (including but not limited to, substitutions, deletions and/or additions) can be naturally produced or artificially introduced without affecting biological functions thereof. Therefore, in the present invention, the term “PD-1 protein” should include all such sequences and natural or artificial variants thereof. Moreover, when describing the sequence fragment of the PD-1 protein, it includes not only the sequence fragment but also a corresponding sequence fragment in natural or artificial variants thereof.

As used herein, when referring to the amino acid sequence of PDL1 protein (NCBI Genebank ID: NP_054862.1), it includes the full length of PDL1 protein, or the extracellular fragment PDL1ECD of PDL1 or a fragment comprising PDL1ECD; also included are fusion proteins of PDL1ECD, such as a fragment fused to an Fc protein fragment of a mouse or human IgG (mFc or hFc). However, those skilled in the art will appreciate that in the amino acid sequence of the PDL1 protein, mutations or variations (including but not limited to, substitutions, deletions and/or additions) can be naturally produced or artificially introduced without affecting biological functions thereof. Therefore, in the present invention, the term “PDL1 protein” shall include all such sequences and natural or artificial variants thereof. Moreover, when describing the sequence fragment of the PDL1 protein, it includes not only a PDL1 sequence fragment but also a corresponding sequence fragment in natural or artificial variants thereof.

As used herein, the term EC₅₀ refers to the half maximum effective concentration.

As used herein, the term “antibody” refers to an immunoglobulin molecule that generally consists of two pairs of polypeptide chains (each pair with one “light” (L) chain and one “heavy” (H) chain). Antibody light chains are classified as κ and λ light chains. Heavy chains are classified as μ, δ, γ, α, or ε. Isotypes of antibodies are defined as IgM, IgD, IgG, IgA, and IgE. In light chains and heavy chains, the variable region and constant region are linked by a “J” region of about 12 or more amino acids, and the heavy chain also comprises a “D” region of about 3 or more amino acids. Each heavy chain consists of a heavy chain variable region (V_{H}) and a heavy chain constant region (C_{H}). The heavy chain constant region consists of 3 domains (C_{H1}, C_{H2}, and C_{H3}). Each light chain consists of a light chain variable region (V_{L}) and a light chain constant region (C_{L}). The light chain constant region consists of one domain C_{L}. The constant region of the antibody can mediate the binding of immunoglobulins to host tissues or factors, including the binding of various cells of the immune system (e.g., effector cells) to the first component (C1q) of classical complement system. The V_{H} and V_{L} regions can be further subdivided into highly variable regions (called complementarity determining regions (CDRs)), between which conservative regions called framework regions (FRs) are distributed. Each V_{H} and V_{L} consists of 3 CDRs and 4 FRs arranged from amino terminus to carboxyl terminus in the following order: FR1, CDR1, FR2, CDR2, FR3, CDR3, FR4. The variable regions (V_{H} and V_{L}) of each heavy chain/light chain pair form an antibody binding site. The assignment of amino acids to each region or domain follows the definition of Kabat Sequences of Proteins of Immunological Interest (National Institutes of Health, Bethesda, Md. (1987 and 1991)), Chothia & Lesk, (1987) J. Mol. Biol., 196:901-917, or Chothia et al. (1989) Nature, 342:878-883. The term “antibody” is not limited by any specific method for producing antibody. For example, the antibody includes, in particular, a recombinant antibody, a monoclonal antibody, and a polyclonal antibody. The antibody can be antibodies of different isotypes, such as IgG (e.g., subtype IgG1, IgG2, IgG3 or IgG4), IgA1, IgA2, IgD, IgE or IgM.

As used herein, the terms “mAb” and “monoclonal antibody” refer to an antibody or a fragment thereof that is derived from a group of highly homologous antibodies, i.e., from a group of identical antibody molecules, except for natural mutations that may occur spontaneously. The monoclonal antibody is highly specific for a single epitope on an antigen. The Polyclonal antibody, relative to the monoclonal antibody, generally comprises at least two or more different antibodies which generally recognize different epitopes on an antigen. Monoclonal antibodies can generally be obtained by hybridoma technique first reported by Kohler et al. (Nature, 256:495, 1975), and can also be obtained by recombinant DNA technique (for example, see U.S. Pat. No. 4,816,567).

As used herein, the term “humanized antibody” refers to an antibody or antibody fragment obtained when all or a part of CDR regions of a human immunoglobulin (receptor antibody) are replaced by the CDR regions of a non-human antibody (donor antibody), wherein the donor antibody may be a non-human (e.g., mouse, rat or rabbit) antibody having expected specificity, affinity or reactivity. In addition, some amino acid residues in the framework regions (FRs) of the receptor antibody can also be replaced by the amino acid residues of corresponding non-human antibodies or by the amino acid residues of other antibodies to further improve or optimize the performance of the antibody. For more details on humanized antibodies, see, for example, Jones et al., Nature, 321:522-525 (1986); Reichmann et al., Nature, 332:323-329 (1988); Presta, Curr. Op. Struct Biol., 2:593-596 (1992); and Clark, Immunol. Today, 21:397-402 (2000).

As used herein, the term “isolated” refers to obtaining by artificial means from natural state. If a certain “isolated” substance or component is present in nature, it may be the case that change occurs in its natural environment, or that it is isolated from the natural environment, or both. For example, if a certain non-isolated polynucleotide or polypeptide naturally exists in a certain living animal, such a polynucleotide or polypeptide with a higher purity isolated from such a natural state is called an isolated polynucleotide or polypeptide. The term “isolated” does not exclude the existence of artificial or synthetic substances or other impurities that do not affect the activity of the substance.

As used herein, the term “vector” refers to a nucleic acid vehicle into which a polynucleotide can be inserted. When a vector allows the expression of the protein encoded by the inserted polynucleotide, the vector is called an expression vector. The vector can be introduced into a host cell by transformation, transduction, or transfection so that the genetic substance elements carried by the vector can be expressed in the host cell. Vectors are well known to those skilled in the art, including but not limited to: plasmids; phagemids; cosmids; artificial chromosomes, such as yeast artificial chromosome (YAC), bacterial artificial chromosome (BAC), or P1-derived artificial chromosome (PAC); phages such as lambda phages or M13 phages; and animal viruses. Animal viruses that can be used as vectors include, but are not limited to retroviruses (including lentiviruses), adenoviruses, adeno-associated viruses, herpes viruses (such as herpes simplex virus), poxviruses, baculoviruses, papillomaviruses, and papovaviruses (such as SV40). A vector may comprise a variety of elements that control expression, including, but not limited to promoter sequences, transcription initiation sequences, enhancer sequences, selection elements, and reporter genes. In addition, the vector may further comprise a replication initiation site.

As used herein, the term “host cell” refers to cells to which vectors can be introduced, including, but not limited to, prokaryotic cells such as E. coli or Bacillus subtilis, fungal cells such as yeast cells or aspergillus, insect cells such as S2 drosophila cells or Sf9, or animal cells such as fibroblasts, CHO cells, COS cells, NSO cells, HeLa cells, BHK cells, HEK 293 cells, or human cells.

As used herein, the term “specific binding” refers to a non-random binding reaction between two molecules, such as a reaction between an antibody and an antigen it targets. In some embodiments, an antibody specifically binding to an antigen (or an antibody specific to an antigen) means that the antibody binds to the antigen with an affinity (K_{D}) of less than about 10⁻⁵ M, e.g., less than about 10⁻⁶ M, 10⁻⁷ M, 10⁻⁸ M, 10⁻⁹ M or 10⁻¹⁰ M or less. As used herein, the term “K_{D}” refers to a dissociation equilibrium constant for a specific antibody-antigen interaction, which is used to describe the binding affinity between the antibody and the antigen. A smaller equilibrium dissociation constant indicates a stronger antibody-antigen binding and a higher affinity between the antibody and the antigen. Generally, antibodies bind to antigens (e.g., PD-1 protein) with a dissociation equilibrium constant (K_{D}) of less than about 10⁻⁵ M, such as less than about 10⁻⁶ M, 10⁻⁷ M, 10⁻⁸ M, 10⁻⁹ M or 10⁻¹⁰ M or less. K_{D} can be determined using methods known to those skilled in the art, e.g., using a Fortebio system.

As used herein, the terms “monoclonal antibody” and “mAb” have the same meaning and can be used interchangeably; the terms “polyclonal antibody” and “pAb” have the same meaning and can be used interchangeably; the terms “polypeptide” and “protein” have the same meaning and can be used interchangeably. Besides, amino acids are generally represented herein by single-letter and three-letter abbreviations known in the art. For example, alanine can be represented by A or Ala.

As used herein, the term “pharmaceutically acceptable carrier and/or excipient” refers to a carrier and/or excipient that is pharmacologically and/or physiologically compatible with the subject and the active ingredient. Such carriers and/or excipients are well known in the art (see, e.g., Remington's Pharmaceutical Sciences, edited by Gennaro A R, 19^{th} Ed., Pennsylvania, Mack Publishing Company, 1995), including but not limited to: pH regulators, surfactants, adjuvants, and ionic strength enhancers. For example, the pH regulators include, but are not limited to, phosphate buffer; the surfactants include, but are not limited to, cationic, anionic, or non-ionic surfactants, such as Tween-80; the ionic strength enhancers include, but are not limited to, sodium chloride.

As used herein, the term “adjuvant” refers to a non-specific immune enhancer, which can enhance the immune response of an organism to antigens or change the type of immune response when delivered into the organism together with the antigens or in advance. There are various adjuvants, including, but not limited to, aluminum adjuvant (e.g., aluminum hydroxide), Freund's adjuvant (e.g., complete Freund's adjuvant and incomplete Freund's adjuvant), Corynebacterium parvum, lipopolysaccharide, cytokine, etc. The Freund's adjuvant is the most commonly used adjuvant in animal experiments. The aluminum hydroxide adjuvant is used more frequently in clinical trials.

As used herein, the term “effective amount” refers to an amount sufficient to obtain or at least partially obtain desired effects. For example, a prophylactically effective amount against a disease (e.g., RA) refers to an amount sufficient to prevent, stop, or delay the onset of the disease (e.g., RA); a therapeutically effective amount refers to an amount sufficient to cure or at least partially stop a disease and complications thereof in patients suffering from the disease. It is undoubtedly within the ability of those skilled in the art to determine such an effective amount. For example, the amount effective for therapeutic purpose will depend on the severity of the disease to be treated, the overall state of the patient's own immune system, the general condition of the patient such as age, body weight and gender, the route of administration, and other treatments given concurrently, etc.

As used herein, the term “completely eliminated” refers to the absence of binding signal or an extremely weak binding signal as detected by existing instrumentation (e.g., a Fortebio Octet system). In one embodiment of the present invention, the absence of binding signal or the extremely weak binding signal refers to a binding signal (i.e., response) below 0.1 nm. A “recurrent” cancer is one that regenerates at the original site or a distant site after response to a previous treatment (e.g., surgery). A “locally recurrent” cancer is one that occurs at the same site as the previously treated cancer after treatment.

A “metastatic” cancer refers to one that spreads from one part of the body (e.g., the lungs) to another.

Beneficial Effects

The present invention achieves one or more of the following technical effects (1) to (9):

(1) The antibodies disclosed herein, in particular 14C12H1L1(hG1TM) and 14C12H1L1(hG1WT), can effectively block the immunosuppression of immune cells induced by PD-1/PDL1 binding, and induce secretion of IFN-γ and IL-2 in human peripheral blood mononuclear cells.

(2) The present invention completely eliminates the binding activity of the antibodies, in particular 14C12H1L1(hG1TM), to Fc receptors, i.e., FcγRI, FcγRIIa_H131, FcγRIIIa_V158 and/or FcγRIIIa_F158, thereby eliminating the ADCC activity or ADCP activity.

(3) The present invention completely eliminates the binding activity of the antibodies, in particular 14C12H1L1(hG1TM), to complement C1q, thereby eliminating the CDC activity.

(4) The present invention significantly reduces the binding activity of the antibodies, e.g., 14C12H1L1 (hG1DM), to Fc receptors, i.e., FcγRI, FcγRIIa_H131, FcγRIIa_R131 and/or FcγRIIIa_V158 and completely eliminates the binding to FcγRIIIa_F158 and/or FcγRIIb, thereby significantly reducing the ADCC activity.

(5) The present invention completely eliminates the binding activity of the antibodies, in particular 14C12H1L1(hG1DM), to complement C1q, thereby eliminating the CDC activity.

(6) The monoclonal antibodies of the present invention, in particular 14C12H1L1(hG1TM), 14C12H1L1(hG1DM) and 14C12H1L1(hG1WT), can be well and specifically bind to PD-1, and can effectively block the binding of PD-1 to PDL1, thereby specifically relieving the immunosuppression by PD-1 in an organism and activating T lymphocytes. Among these, the PD-1 antibody 14C12H1L1(hG1TM) has an significantly stronger induction effect than those of the control anti-PD-1 antibody nivolumab and the control anti-PDL1 antibody 5C10H2L2-IgG1mt on IFN-γ and IL-2 secretion, showing potential for use in preparing a medicament for preventing and treating tumors.

(7) The antibodies disclosed herein have ability to effectively prevent and treat the tumors described above.

(8) The antibodies disclosed herein have lower toxic and side effects.

(9) The anti-PD-1 antibodies disclosed herein or the anti-PD-1 antibodies in the therapeutic combination disclosed herein have a synergistic effect with a chemotherapeutic.

### BRIEF DESCRIPTION OF THE DRAWINGS

FIG. 1: Affinity constant assay of 14C12H1L1(hG1DM) to FcγRI. The antibody concentrations for the curve pairs from top to bottom are 50 nM, 25 nM, 12.5 nM, 6.25 nM and 3.12 nM, respectively.

FIG. 2: Affinity constant assay of 14C12H1L1(hG4) to FcγRI. The antibody concentrations for the curve pairs from top to bottom are 50 nM, 25 nM, 12.5 nM, 6.25 nM and 3.12 nM, respectively.

FIG. 3: Affinity constant assay of 14C12H1L1(hG1WT) to FcγRI. The antibody concentrations for the curve pairs from top to bottom are 50 nM, 25 nM, 12.5 nM, 6.25 nM and 3.12 nM, respectively.

FIG. 4: Affinity constant assay of 14C12H1L1(hG1TM) to FcγRI. The antibody concentrations for the curve pairs from top to bottom are 50 nM, 25 nM, 12.5 nM, 6.25 nM and 3.12 nM, respectively.

FIG. 5: Affinity constant assay of 5C10H2L2-IgG1mt to FcγRI. The antibody concentrations for the curve pairs from top to bottom are 50 nM, 25 nM, 12.5 nM, 6.25 nM and 3.12 nM, respectively.

FIG. 6: Affinity constant assay of 14C12H1L1(hG1DM) to FcγRIIIa_V158. The antibody concentrations for the curve pairs from top to bottom are 500 nM, 250 nM, 125 nM, 62.5 nM and 31.25 nM, respectively.

FIG. 7: Affinity constant assay of 14C12H1L1(hG4) to FcγRIIIa_V158. The antibody concentrations for the curve pairs from top to bottom are 500 nM, 250 nM, 125 nM, 62.5 nM and 31.25 nM, respectively.

FIG. 8: Affinity constant assay of 14C12H1L1(hG1WT) to FcγRIIIa_V158. The antibody concentrations for the curve pairs from top to bottom are 500 nM, 250 nM, 125 nM, 62.5 nM and 31.25 nM, respectively.

FIG. 9: Affinity constant assay of 14C12H1L1(hG1TM) to FcγRIIIa_V158. The antibody concentrations for the curve pairs from top to bottom are 500 nM, 250 nM, 125 nM, 62.5 nM and 31.25 nM, respectively.

FIG. 10: Affinity constant assay of 5C10H2L2-IgG1mt to FcγRIIIa_V158. The antibody concentrations for the curve pairs from top to bottom are 500 nM, 250 nM, 125 nM, 62.5 nM and 31.25 nM, respectively.

FIG. 11: Affinity constant assay of 14C12H1L1(hG1DM) to FcγRIIIa_F158. The antigen concentrations for the curve pairs from top to bottom are 500 nM, 250 nM, 125 nM, 62.5 nM and 31.25 nM, respectively.

FIG. 12: Affinity constant assay of 14C12H1L1(hG4) to FcγRIIIa_F158. The antibody concentrations for the curve pairs from top to bottom are 500 nM, 250 nM, 125 nM, 62.5 nM and 31.25 nM, respectively.

FIG. 13: Affinity constant assay of 14C12H1L1(hG1WT) to FcγRIIIa_F158. The antibody concentrations for the curve pairs from top to bottom are 500 nM, 250 nM, 125 nM, 62.5 nM and 31.25 nM, respectively.

FIG. 14: Affinity constant assay of 14C12H1L1(hG1TM) to FcγRIIIa_F158. The antibody concentrations for the curve pairs from top to bottom are 500 nM, 250 nM, 125 nM, 62.5 nM and 31.25 nM, respectively.

FIG. 15: Affinity constant assay of 5C10H2L2-IgG1mt to FcγRIIa_F158. The antibody concentrations for the curve pairs from top to bottom are 500 nM, 250 nM, 125 nM, 62.5 nM and 31.25 nM, respectively.

FIG. 16: Affinity constant assay of 14C12H1L1(hG1DM) to FcγRIIa_H131. The antibody concentrations for the curve pairs from top to bottom are 200 nM, 100 nM, 50 nM, 25 nM and 12.5 nM, respectively.

FIG. 17: Affinity constant assay of 14C12H1L1(hG4) to FcγRIIa_H131. The antibody concentrations for the curve pairs from top to bottom are 200 nM, 100 nM, 50 nM, 25 nM and 12.5 nM, respectively.

FIG. 18: Affinity constant assay of 14C12H1L1(hG1WT) to FcγRIIa_H131. The antibody concentrations for the curve pairs from top to bottom are 200 nM, 100 nM, 50 nM, 25 nM and 12.5 nM, respectively.

FIG. 19: Affinity constant assay of 14C12H1L1(hG1TM) to FcγRIIa_H131. The antibody concentrations for the curve pairs from top to bottom are 200 nM, 100 nM, 50 nM, 25 nM and 12.5 nM, respectively.

FIG. 20: Affinity constant assay of 5C10H2L2-IgG1mt to FcγRIIa_H131. The antibody concentrations for the curve pairs from top to bottom are 200 nM, 100 nM, 50 nM, 25 nM and 12.5 nM, respectively.

FIG. 21: Affinity constant assay of 14C12H1L1(hG1DM) to FcγRIIa_R131. The antibody concentrations for the curve pairs from top to bottom are 200 nM, 100 nM, 50 nM, 25 nM and 12.5 nM, respectively.

FIG. 22: Affinity constant assay of 14C12H1L1(hG4) to FcγRIIa_R131. The antibody concentrations for the curve pairs from top to bottom are 200 nM, 100 nM, 50 nM, 25 nM and 12.5 nM, respectively.

FIG. 23: Affinity constant assay of 14C12H1L1(hG1WT) to FcγRIIa_R131. The antibody concentrations for the curve pairs from top to bottom are 200 nM, 100 nM, 50 nM, 25 nM and 12.5 nM, respectively.

FIG. 24: Affinity constant assay of 14C12H1L1(hG1TM) to FcγRIIa_R131. The antibody concentrations for the curve pairs from top to bottom are 200 nM, 100 nM, 50 nM, 25 nM and 12.5 nM, respectively.

FIG. 25: Affinity constant assay of 5C10H2L2-IgG1mt to FcγRIIa_R131. The antibody concentrations for the curve pairs from top to bottom are 200 nM, 100 nM, 50 nM, 25 nM and 12.5 nM, respectively.

FIG. 26: Affinity constant assay of 14C12H1L1(hG1DM) to FcγRIIb. The antibody concentrations for the curve pairs from top to bottom are 200 nM, 100 nM, 50 nM, 25 nM and 12.5 nM, respectively.

FIG. 27: Affinity constant assay of 14C12H1L1(hG4) to FcγRIIb. The antibody concentrations for the curve pairs from top to bottom are 200 nM, 100 nM, 50 nM, 25 nM and 12.5 nM, respectively.

FIG. 28: Affinity constant assay of 14C12H1L1(hG1WT) to FcγRIIb. The antibody concentrations for the curve pairs from top to bottom are 200 nM, 100 nM, 50 nM, 25 nM and 12.5 nM, respectively.

FIG. 29: Affinity constant assay of 14C12H1L1(hG1TM) to FcγRIIb. The antibody concentrations for the curve pairs from top to bottom are 200 nM, 100 nM, 50 nM, 25 nM and 12.5 nM, respectively.

FIG. 30: Affinity constant assay of 5C10H2L2-IgG1mt to FcγRIIb. The antibody concentrations for the curve pairs from top to bottom are 200 nM, 100 nM, 50 nM, 25 nM and 12.5 nM, respectively.

FIG. 31: Affinity constant assay of 14C12H1L1(hG1DM) to C1q. The antibody concentrations for the curve pairs from top to bottom are 20 nM, 10 nM, 5 nM, 2.5 nM and 1.25 nM, respectively.

FIG. 32: Affinity constant assay of 14C12H1L1(hG4) to C1q. The antibody concentrations for the curve pairs from top to bottom are 20 nM, 10 nM, 5 nM, 2.5 nM and 1.25 nM, respectively.

FIG. 33: Affinity constant assay of 14C12H1L1(hG1WT) to C1q. The antibody concentrations for the curve pairs from top to bottom are 20 nM, 10 nM, 5 nM, 2.5 nM and 1.25 nM, respectively.

FIG. 34: Affinity constant assay of 14C12H1L1(hG1TM) to C1q. The antibody concentrations for the curve pairs from top to bottom are 20 nM, 10 nM, 5 nM, 2.5 nM and 1.25 nM, respectively.

FIG. 35: Affinity constant assay of 5C10H2L2-IgG1mt to C1q. The antigen concentrations for the curve pairs from top to bottom are 20 nM, 10 nM, 5 nM, 2.5 nM and 1.25 nM, respectively.

FIG. 36: IFN-γ secretion assay by adding 14C12H1L1 (hG1WT) and 14C12H1L1(hG1TM) to mixed lymphocyte reaction.

FIG. 37: IL-2 secretion assay by adding 14C12H1L1 (hG1WT) and 14C12H1L1(hG1TM) to mixed lymphocyte reaction.

FIG. 38: ADCP effect assay of 14C12H1L1(hG1WT), nivolumab, and 14C12H1L1(hG1TM).

FIG. 39: Killing effect assay of 14C12H1L1(hG1TM)+anlotinib on human non-small cell lung cancer cells.

FIG. 40: Inhibited proliferation of mouse colorectal cancer MC38 cells by 14C12H1L1(hG1TM).

FIG. 41: Effectively enhanced immune response of immune cells to human gastric cancer KATO III cells by 14C12H1L1(hG1TM).

FIG. 42: Effectively enhanced immune response of immune cells to nasopharyngeal cancer CNE-2Z cells by 14C12H1L1(hG1TM).

FIG. 43: Effectively enhanced immune response of immune cells to mesothelioma NCI-H2452 cells by 14C12H1L1(hG1TM).

FIG. 44: Effectively enhanced immune response of immune cells to human non-small cell lung cancer NCI-11446 cells by 14C12H1L1(hG1TM).

FIG. 45: Effectively enhanced immune response of immune cells to nasopharyngeal cancer CNE-2Z cells by 14C12H1L1(hG1TM) in combination with anlotinib hydrochloride.

FIG. 46: Significantly enhanced immune response of immune cells to MSI-H/dMMR tumor SW48 cells by 14C12H1L1(hG1TM) in combination with anlotinib.

FIG. 47: Significantly enhanced immune response of immune cells to human colorectal cancer SW837 cells of non-MSI-H/dMMR (i.e., MSS) phenotype by 14C12H1L1(hG1TM).

FIG. 48: Significantly enhanced immune response of immune cells to human colorectal cancer SW837 cells of non-MSI-H/dMMR (i.e., MSS) phenotype by 14C12H1L1(hG1TM) in combination with anlotinib.

### DETAILED DESCRIPTION

The embodiments of the present invention will be described in detail below with reference to the examples. Those skilled in the art will understand that the following examples are only for illustrating the present invention, and should not be construed as limitation on the scope of the present invention. In the cases where the techniques or conditions are not specified, the examples were implemented according to the techniques or conditions described in the literature in the art (e.g., see, Molecular Cloning: A Laboratory Manual, authored by J. Sambrook et al., and translated by Huang Peitang et al., Third Edition, Science Press) or according to the product manual. Reagents or instruments used are commercially available conventional products if the manufacturers thereof are not specified.

In the following experiments of the present invention:

BALB/c mice were purchased from Guangdong Medical Laboratory Animal Center.

The anti-PDL1 antibody 5C10H2L2-IgG1mt was prepared by methods described in PCT Publication No. WO2017148424A1.

The anti-PD-1 antibody nivolumab (trade name: Opdivo) was purchased from the Bristol-Myers Squibb.

Human peripheral blood mononuclear cells were isolated and prepared in Akeso Biopharma, Inc., with informed consent of the donor.

Raji-PDL1 is a cell expressing human PD-L1 constructed by Akeso Biopharma on the basis of human B cells Raji via transfection.

Ficoll-Paque™ PLUS (or Ficoll-Paque PLUS) was purchased from GE Healthcare.

Human IL-2 ELISA kit was purchased from Dakewe Biotech Co., Ltd.

RPMI 1640 medium, DMEM medium, Trypsin-EDTA (0.25%) phenol red and Blastidin were all purchased from Gibco.

Staphylococcus aureus enterotoxin B (SEB) was purchased from Dianotech.

FBS was purchased from Excell bio.

Mitomycin C (MMC) was purchased from Stressmarq.

The sequence of the isotype control, human anti-hen egg lysozyme IgG (anti-HEL antibody, or human IgG, abbreviated as hIgG) is derived from the variable region sequence of the Fab F10.6.6 sequence in the study reported by Acierno et al., entitled “Affinity maturation increases the stability and plasticity of the Fv domain of anti-protein antibodies” (Acierno et al., J Mol Biol., 2007; 374(1):130-146).

Anlotinib used in the examples is hydrochloride salt of anlotinib under the brand name Fukewei® and generic name anlotinib hydrochloride, and was purchased from CTTQ Pharma.

### Preparation Example 1: Sequence Design of Anti-PD-1 Antibody 14C12 and its Humanized Antibody 14C12H1L1(hG1WT)

The amino acid sequences and encoding nucleotide sequences of the heavy and light chains of anti-PD-1 antibody 14C12 and its humanized antibody 14C12H1L1(hG1WT) are identical to those of 14C12 and 14C12H1L1 in Chinese Patent Publication No. CN106967172A (or No. CN106977602A), respectively.

(1) Heavy and Light Chain Variable Region Sequences of 14C12

Nucleotide sequence of the heavy chain variable
region of 14C12: (354 bp)
(SEQ ID NO: 1)
GAGGTCAAACTGGTGGAGAGCGGCGGCGGGCTGGTGAAGCCCGGCGGGT
CACTGAAACTGAGCTGCGCCGCTTCCGGCTTCGCCTTTAGCTCCTACGA
CATGTCATGGGTGAGGCAGACCCCTGAGAAGCGCCTGGAATGGGTCGCT
ACTATCAGCGGAGGCGGGCGATACACCTACTATCCTGACTCTGTCAAAG
GGAGATTCACAATTAGTCGGGATAACGCCAGAAATACTCTGTATCTGCA
GATGTCTAGTCTGCGGTCCGAGGATACAGCTCTGTACTATTGTGCAAAC
CGGTACGGCGAAGCATGGTTTGCCTATTGGGGACAGGGCACCCTGGTGA
CAGTCTCTGCC
Amino acid sequence of the heavy chain variable
region of 14C12: (118 aa)
(SEQ ID NO: 2)
EVKLVESGGGLVKPGGSLKLSCAASGFAFSSYDMSWVRQTPEKRLEWVA
TISGGGRYTYYPDSVKGRFTISRDNARNTLYLQMSSLRSEDTALYYCAN
RYGEAWFAYWGQGTLVTVSA
Nucleotide sequence encoding the light chain
variable region of 14C12: (321 bp)
(SEQ ID NO: 3)
GACATTAAGATGACACAGTCCCCTTCCTCAATGTACGCTAGCCTGGGCG
AGCGAGTGACCTTCACATGCAAAGCATCCCAGGACATCAACACATACCT
GTCTTGGTTTCAGCAGAAGCCAGGCAAAAGCCCCAAGACCCTGATCTAC
CGGGCCAATAGACTGGTGGACGGGGTCCCCAGCAGATTCTCCGGATCTG
GCAGTGGGCAGGATTACTCCCTGACCATCAGCTCCCTGGAGTATGAAGA
CATGGGCATCTACTATTGCCTGCAGTATGATGAGTTCCCTCTGACCTTT
GGAGCAGGCACAAAACTGGAACTGAAG
Amino acid sequence of the light chain variable
region of 14C12: (107 aa)
(SEQ ID NO: 4)
DIKMTQSPSSMYASLGERVTFTCKASQDINTYLSWFQQKPGKSPKTLIY
RANRLVDGVPSRFSGSGSGQDYSLTISSLEYEDMGIYYCLQYDEFPLTF
GAGTKLELK

(2) Heavy and Light Chain Variable Region and Heavy and Light Chain Sequences of Humanized Monoclonal Antibody 14C12H1L1(hG1WT)

Nucleotide sequence of the heavy chain variable
region of 14C12H1L1(hG1WT): (354 bp)
(SEQ ID NO: 5)
GAAGTGCAGCTGGTCGAGTCTGGGGGAGGGCTGGTGCAGCCCGGCGGGT
CACTGCGACTGAGCTGCGCAGCTTCCGGATTCGCCTTTAGCTCCTACGA
CATGTCCTGGGTGCGACAGGCACCAGGAAAGGGACTGGATTGGGTCGCT
ACTATCTCAGGAGGCGGGAGATACACCTACTATCCTGACAGCGTCAAGG
GCCGGTTCACAATCTCTAGAGATAACAGTAAGAACAATCTGTATCTGCA
GATGAACAGCCTGAGGGCTGAGGACACCGCACTGTACTATTGTGCCAAC
CGCTACGGGGAAGCATGGTTTGCCTATTGGGGGCAGGGAACCCTGGTGA
CAGTCTCTAGT
Amino acid sequence of the heavy chain variable
region of 14C12H1L1(hG1WT): (118 aa)
(SEQ ID NO: 6)
EVQLVESGGGLVQPGGSLRLSCAASGFAFSSYDMSWVRQAPGKGLDWVA
TISGGGRYTYYPDSVKGRFTISRDNSKNNLYLQMNSLRAEDTALYYCAN
RYGEAWFAYWGQGTLVTVSS
Nucleotide sequence encoding the light chain
variable region of 14C12H1L1(hG1WT): (321 bp)
(SEQ ID NO: 7)
GACATTCAGATGACTCAGAGCCCCTCCTCCATGTCCGCCTCTGTGGGCG
ACAGGGTCACCTTCACATGCCGCGCTAGTCAGGATATCAACACCTACCT
GAGCTGGTTTCAGCAGAAGCCAGGGAAAAGCCCCAAGACACTGATCTAC
CGGGCTAATAGACTGGTGTCTGGAGTCCCAAGTCGGTTCAGTGGCTCAG
GGAGCGGACAGGACTACACTCTGACCATCAGCTCCCTGCAGCCTGAGGA
CATGGCAACCTACTATTGCCTGCAGTATGATGAGTTCCCACTGACCTTT
GGCGCCGGGACAAAACTGGAGCTGAAG
Amino acid sequence of the light chain variable
region of 14C12H1L1(hG1WT): (107 aa)
(SEQ ID NO: 8)
DIQMTQSPSSMSASVGDRVTFTCRASQDINTYLSWFQQKPGKSPKTLIY
RANRLVSGVPSRFSGSGSGQDYTLTISSLQPEDMATYYCLQYDEFPLTF
GAGTKLELK
Nucleotide sequence of the heavy chain of
14C12H1L1(hG1WT): (1344 bp)
(SEQ ID NO: 9)
GAAGTGCAGCTGGTCGAGTCTGGGGGAGGGCTGGTGCAGCCCGGCGGGT
CACTGCGACTGAGCTGCGCAGCTTCCGGATTCGCCTTTAGCTCCTACGA
CATGTCCTGGGTGCGACAGGCACCAGGAAAGGGACTGGATTGGGTCGCT
ACTATCTCAGGAGGCGGGAGATACACCTACTATCCTGACAGCGTCAAGG
GCCGGTTCACAATCTCTAGAGATAACAGTAAGAACAATCTGTATCTGCA
GATGAACAGCCTGAGGGCTGAGGACACCGCACTGTACTATTGTGCCAAC
CGCTACGGGGAAGCATGGTTTGCCTATTGGGGGCAGGGAACCCTGGTGA
CAGTCTCTAGTGCCAGCACCAAAGGACCTAGCGTGTTTCCTCTCGCCCC
CTCCTCCAAAAGCACCAGCGGAGGAACCGCTGCTCTCGGATGTCTGGTG
AAGGACTACTTCCCTGAACCCGTCACCGTGAGCTGGAATAGCGGCGCTC
TGACAAGCGGAGTCCATACATTCCCTGCTGTGCTGCAAAGCAGCGGACT
CTATTCCCTGTCCAGCGTCGTCACAGTGCCCAGCAGCAGCCTGGGCACC
CAGACCTACATCTGTAACGTCAACCACAAGCCCTCCAACACCAAGGTGG
ACAAGAAAGTGGAGCCCAAATCCTGCGACAAGACACACACCTGTCCCCC
CTGTCCTGCTCCCGAACTCCTCGGAGGCCCTAGCGTCTTCCTCTTTCCT
CCCAAACCCAAGGACACCCTCATGATCAGCAGAACCCCTGAAGTCACCT
GTGTCGTCGTGGATGTCAGCCATGAGGACCCCGAGGTGAAATTCAACTG
GTATGTCGATGGCGTCGAGGTGCACAACGCCAAAACCAAGCCCAGGGAG
GAACAGTACAACTCCACCTACAGGGTGGTGTCCGTGCTGACAGTCCTCC
ACCAGGACTGGCTGAACGGCAAGGAGTACAAGTGCAAGGTGTCCAACAA
GGCTCTCCCTGCCCCCATTGAGAAGACCATCAGCAAGGCCAAAGGCCAA
CCCAGGGAGCCCCAGGTCTATACACTGCCTCCCTCCAGGGACGAACTCA
CCAAGAACCAGGTGTCCCTGACCTGCCTGGTCAAGGGCTTTTATCCCAG
CGACATCGCCGTCGAGTGGGAGTCCAACGGACAGCCCGAGAATAACTAC
AAGACCACCCCTCCTGTCCTCGACTCCGACGGCTCCTTCTTCCTGTACA
GCAAGCTGACCGTGGACAAAAGCAGGTGGCAGCAGGGAAACGTGTTCTC
CTGCAGCGTGATGCACGAAGCCCTCCACAACCACTACACCCAGAAAAGC
CTGTCCCTGAGCCCCGGCAAA
Amino acid sequence of the heavy chain of
14C12H1L1(hG1WT): (448 aa)
(SEQ ID NO: 10)
EVQLVESGGGLVQPGGSLRLSCAASGFAFSSYDMSWVRQAPGKGLDWVA
TISGGGRYTYYPDSVKGRFTISRDNSKNNLYLQMNSLRAEDTALYYCAN
RYGEAWFAYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK
Nucleotide sequence of the light chain of
14C12H1L1(hG1WT): (642 bp)
(SEQ ID NO: 11)
GACATTCAGATGACTCAGAGCCCCTCCTCCATGTCCGCCTCTGTGGGCG
ACAGGGTCACCTTCACATGCCGCGCTAGTCAGGATATCAACACCTACCT
GAGCTGGTTTCAGCAGAAGCCAGGGAAAAGCCCCAAGACACTGATCTAC
CGGGCTAATAGACTGGTGTCTGGAGTCCCAAGTCGGTTCAGTGGCTCAG
GGAGCGGACAGGACTACACTCTGACCATCAGCTCCCTGCAGCCTGAGGA
CATGGCAACCTACTATTGCCTGCAGTATGATGAGTTCCCACTGACCTTT
GGCGCCGGGACAAAACTGGAGCTGAAGCGAACTGTGGCCGCTCCCTCCG
TCTTCATTTTTCCCCCTTCTGACGAACAGCTGAAATCAGGCACAGCCAG
CGTGGTCTGTCTGCTGAACAATTTCTACCCTAGAGAGGCAAAAGTGCAG
TGGAAGGTCGATAACGCCCTGCAGTCCGGCAACAGCCAGGAGAGTGTGA
CTGAACAGGACTCAAAAGATAGCACCTATTCCCTGTCTAGTACACTGAC
TCTGTCCAAGGCTGATTACGAGAAGCACAAAGTGTATGCATGCGAAGTG
ACACATCAGGGACTGTCAAGCCCCGTGACTAAGTCTTTTAACCGGGGCG
AATGT
Amino acid sequence of the light chain of
14C12H1L1(hG1WT): (214 aa)
(SEQ ID NO: 12)
DIQMTQSPSSMSASVGDRVTFTCRASQDINTYLSWFQQKPGKSPKTLIY
RANRLVSGVPSRFSGSGSGQDYTLTISSLQPEDMATYYCLQYDEFPLTF
GAGTKLELKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

### Preparation Example 2: Sequence Design of Humanized Antibody 14C12H1L1(hG4)

The heavy and light chain variable regions are identical to those of 14C12H1L1(hG1WT). Ig gamma-4 chain C region (ACCESSION: P01861.1) was used as the heavy chain constant region, and Ig kappa chain C region (ACCESSION: P01834) was used as the light chain constant region, thus giving the antibody 14C12H1L1(hG4). The sequence of 14C12H1L1(hG4) is as follows:

Nucleotide sequence of the heavy chain of
14C12H1L1(hG4): (1335 bp)
(SEQ ID NO: 13)
GAAGTGCAGCTGGTCGAGTCTGGGGGAGGGCTGGTGCAGCCCGGCGGGT
CACTGCGACTGAGCTGCGCAGCTTCCGGATTCGCCTTTAGCTCCTACGA
CATGTCCTGGGTGCGACAGGCACCAGGAAAGGGACTGGATTGGGTCGCT
ACTATCTCAGGAGGCGGGAGATACACCTACTATCCTGACAGCGTCAAGG
GCCGGTTCACAATCTCTAGAGATAACAGTAAGAACAATCTGTATCTGCA
GATGAACAGCCTGAGGGCTGAGGACACCGCACTGTACTATTGTGCCAAC
CGCTACGGGGAAGCATGGTTTGCCTATTGGGGGCAGGGAACCCTGGTGA
CAGTCTCTAGTGCCAGCACCAAAGGGCCCTCGGTCTTCCCCCTGGCGCC
CTGCTCCAGGAGCACCTCCGAGAGCACAGCCGCCCTGGGCTGCCTGGTC
AAGGACTACTTCCCCGAACCGGTGACGGTGTCGTGGAACTCAGGCGCCC
TGACCAGCGGCGTGCACACCTTCCCGGCTGTCCTACAGTCCTCAGGACT
CTACTCCCTCAGCAGCGTGGTGACCGTGCCCTCCAGCAGCTTGGGCACG
AAGACCTACACCTGCAACGTAGATCACAAGCCCAGCAACACCAAGGTGG
ACAAGAGAGTTGAGTCCAAATATGGTCCCCCATGCCCACCATGCCCAGC
ACCTGAGTTCCTGGGGGGACCATCAGTCTTCCTGTTCCCCCCAAAACCC
AAGGACACTCTCATGATCTCCCGGACCCCTGAGGTCACGTGCGTGGTGG
TGGACGTGAGCCAGGAAGACCCCGAGGTCCAGTTCAACTGGTACGTGGA
TGGCGTGGAGGTGCATAATGCCAAGACAAAGCCGCGGGAGGAGCAGTTC
AACAGCACGTACCGTGTGGTCAGCGTCCTCACCGTCCTGCACCAGGACT
GGCTGAACGGCAAGGAGTACAAGTGCAAGGTCTCCAACAAAGGCCTCCC
GTCCTCCATCGAGAAAACCATCTCCAAAGCCAAAGGGCAGCCCCGAGAG
CCACAGGTGTACACCCTGCCCCCATCCCAGGAGGAGATGACCAAGAACC
AGGTCAGCCTGACCTGCCTGGTCAAAGGCTTCTACCCCAGCGACATCGC
CGTGGAGTGGGAGAGCAATGGGCAGCCGGAGAACAACTACAAGACCACG
CCTCCCGTGCTGGACTCCGACGGCTCCTTCTTCCTCTACAGCAGGCTAA
CCGTGGACAAGAGCAGGTGGCAGGAGGGGAATGTCTTCTCATGCTCCGT
GATGCATGAGGCTCTGCACAACCACTACACACAGAAGAGCCTCTCCCTG
TCTCTGGGTAAA
Amino acid sequence of the heavy chain of
14C12H1L1(hG4): (445 aa)
(SEQ ID NO: 14)
EVQLVESGGGLVQPGGSLRLSCAASGFAFSSYDMSWVRQAPGKGLDWVA
TISGGGRYTYYPDSVKGRFTISRDNSKNNLYLQMNSLRAEDTALYYCAN
RYGEAWFAYWGQGTLVTVSSASTKGPSVFPLAPCSRSTSESTAALGCLV
KDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLGK

The nucleotide sequence of the 14C12H1L1(hG4) light chain is identical to SEQ ID NO: 11.

The amino acid sequence of the 14C12H1L1(hG4) light chain is identical to SEQ ID NO: 12.

### Preparation Example 3: Sequence Design of Humanized Antibody 14C12H1L1(hG1TM)

On the basis of 14C12H1L1(hG1WT) obtained in Preparation Example 1, a humanized mutant 14C12H1L1(hG1TM) was obtained by introducing a leucine-to-alanine point mutation at position 234 (L234A), a leucine-to-alanine point mutation at position 235 (L235A), and a glycine-to-alanine point mutation at position 237 (G237A) in the hinge region of the heavy chain according to the EU numbering system.

Nucleotide sequence of the heavy chain of
14C12H1L1(hG1TM): (1344 bp)
(SEQ ID NO: 15)
GAAGTGCAGCTGGTCGAGTCTGGGGGAGGGCTGGTGCAGCCCGGCGGGT
CACTGCGACTGAGCTGCGCAGCTTCCGGATTCGCCTTTAGCTCCTACGA
CATGTCCTGGGTGCGACAGGCACCAGGAAAGGGACTGGATTGGGTCGCT
ACTATCTCAGGAGGCGGGAGATACACCTACTATCCTGACAGCGTCAAGG
GCCGGTTCACAATCTCTAGAGATAACAGTAAGAACAATCTGTATCTGCA
GATGAACAGCCTGAGGGCTGAGGACACCGCACTGTACTATTGTGCCAAC
CGCTACGGGGAAGCATGGTTTGCCTATTGGGGGCAGGGAACCCTGGTGA
CAGTCTCTAGTGCCAGCACCAAAGGGCCCAGCGTGTTTCCTCTCGCCCC
CTCCTCCAAAAGCACCAGCGGAGGAACCGCTGCTCTCGGATGTCTGGTG
AAGGACTACTTCCCTGAACCCGTCACCGTGAGCTGGAATAGCGGCGCTC
TGACAAGCGGAGTCCATACATTCCCTGCTGTGCTGCAAAGCAGCGGACT
CTATTCCCTGTCCAGCGTCGTCACAGTGCCCAGCAGCAGCCTGGGCACC
CAGACCTACATCTGTAACGTCAACCACAAGCCCTCCAACACCAAGGTGG
ACAAGAAAGTGGAGCCCAAATCCTGCGACAAGACACACACCTGTCCCCC
CTGTCCTGCTCCCGAAGCTGCTGGAGCCCCTAGCGTCTTCCTCTTTCCT
CCCAAACCCAAGGACACCCTCATGATCAGCAGAACCCCTGAAGTCACCT
GTGTCGTCGTGGATGTCAGCCATGAGGACCCCGAGGTGAAATTCAACTG
GTATGTCGATGGCGTCGAGGTGCACAACGCCAAAACCAAGCCCAGGGAG
GAACAGTACAACTCCACCTACAGGGTGGTGTCCGTGCTGACAGTCCTCC
ACCAGGACTGGCTGAACGGCAAGGAGTACAAGTGCAAGGTGTCCAACAA
GGCTCTCCCTGCCCCCATTGAGAAGACCATCAGCAAGGCCAAAGGCCAA
CCCAGGGAGCCCCAGGTCTATACACTGCCTCCCTCCAGGGACGAACTCA
CCAAGAACCAGGTGTCCCTGACCTGCCTGGTCAAGGGCTTTTATCCCAG
CGACATCGCCGTCGAGTGGGAGTCCAACGGACAGCCCGAGAATAACTAC
AAGACCACCCCTCCTGTCCTCGACTCCGACGGCTCCTTCTTCCTGTACA
GCAAGCTGACCGTGGACAAAAGCAGGTGGCAGCAGGGAAACGTGTTCTC
CTGCAGCGTGATGCACGAAGCCCTCCACAACCACTACACCCAGAAAAGC
CTGTCCCTGAGCCCCGGCAAA
Amino acid sequence of the heavy chain of
14C12H1L1(hG1TM): (448 aa)
(SEQ ID NO: 16)
EVQLVESGGGLVQPGGSLRLSCAASGFAFSSYDMSWVRQAPGKGLDWVA
TISGGGRYTYYPDSVKGRFTISRDNSKNNLYLQMNSLRAEDTALYYCAN
RYGEAWFAYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPEAAGAPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK

The nucleotide sequence of the 14C12H1L1(hG1TM) light chain is identical to SEQ ID NO: 11.

The amino acid sequence of the 14C12H1L1(hG1TM) light chain is identical to SEQ ID NO: 12.

### Preparation Example 4: Sequence Design of Humanized Antibody 14C12H1L1(hG1DM)

On the basis of 14C12H1L1(hG1WT), a humanized mutant antibody 14C12H1L1(hG1DM) was obtained by introducing a leucine-to-alanine point mutation at position 234 (L234A) and a leucine-to-alanine point mutation at position 235 (L235A) in the hinge region of the heavy chain.

Nucleotide sequence of the heavy chain of
14C12H1L1(hG1DM): (1344 bp)
(SEQ ID NO: 17)
GAAGTGCAGCTGGTCGAGTCTGGGGGAGGGCTGGTGCAGCCCGGCGGGT
CACTGCGACTGAGCTGCGCAGCTTCCGGATTCGCCTTTAGCTCCTACGA
CATGTCCTGGGTGCGACAGGCACCAGGAAAGGGACTGGATTGGGTCGCT
ACTATCTCAGGAGGCGGGAGATACACCTACTATCCTGACAGCGTCAAGG
GCCGGTTCACAATCTCTAGAGATAACAGTAAGAACAATCTGTATCTGCA
GATGAACAGCCTGAGGGCTGAGGACACCGCACTGTACTATTGTGCCAAC
CGCTACGGGGAAGCATGGTTTGCCTATTGGGGGCAGGGAACCCTGGTGA
CAGTCTCTAGTGCTAGCACCAAAGGGCCCAGCGTGTTTCCTCTCGCCCC
CTCCTCCAAAAGCACCAGCGGAGGAACCGCTGCTCTCGGATGTCTGGTG
AAGGACTACTTCCCTGAACCCGTCACCGTGAGCTGGAATAGCGGCGCTC
TGACAAGCGGAGTCCATACATTCCCTGCTGTGCTGCAAAGCAGCGGACT
CTATTCCCTGTCCAGCGTCGTCACAGTGCCCAGCAGCAGCCTGGGCACC
CAGACCTACATCTGTAACGTCAACCACAAGCCCTCCAACACCAAGGTGG
ACAAGAAAGTGGAGCCCAAATCCTGCGACAAGACACACACCTGTCCCCC
CTGTCCTGCTCCCGAAGCTGCTGGAGGCCCTAGCGTCTTCCTCTTTCCT
CCCAAACCCAAGGACACCCTCATGATCAGCAGAACCCCTGAAGTCACCT
GTGTCGTCGTGGATGTCAGCCATGAGGACCCCGAGGTGAAATTCAACTG
GTATGTCGATGGCGTCGAGGTGCACAACGCCAAAACCAAGCCCAGGGAG
GAACAGTACAACTCCACCTACAGGGTGGTGTCCGTGCTGACAGTCCTCC
ACCAGGACTGGCTGAACGGCAAGGAGTACAAGTGCAAGGTGTCCAACAA
GGCTCTCCCTGCCCCCATTGAGAAGACCATCAGCAAGGCCAAAGGCCAA
CCCAGGGAGCCCCAGGTCTATACACTGCCTCCCTCCAGGGACGAACTCA
CCAAGAACCAGGTGTCCCTGACCTGCCTGGTCAAGGGCTTTTATCCCAG
CGACATCGCCGTCGAGTGGGAGTCCAACGGACAGCCCGAGAATAACTAC
AAGACCACCCCTCCTGTCCTCGACTCCGACGGCTCCTTCTTCCTGTACA
GCAAGCTGACCGTGGACAAAAGCAGGTGGCAGCAGGGAAACGTGTTCTC
CTGCAGCGTGATGCACGAAGCCCTCCACAACCACTACACCCAGAAAAGC
CTGTCCCTGAGCCCCGGCAAA
Amino acid sequence of the heavy chain of
14C12H1L1(hG1DM): (448 aa)
(SEQ ID NO: 18)
EVQLVESGGGLVQPGGSLRLSCAASGFAFSSYDMSWVRQAPGKGLDWVA
TISGGGRYTYYPDSVKGRFTISRDNSKNNLYLQMNSLRAEDTALYYCAN
RYGEAWFAYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK

The nucleotide sequence of the 14C12H1L1(hG1DM) light chain is identical to SEQ ID NO: 11.

The amino acid sequence of the 14C12H1L1(hG1DM) light chain is identical to SEQ ID NO: 12.

### Experimental Example 1: Affinity Assay of 14C12H1L1(hG1DM), 14C12H1L1(hG4), 14C12H1L1(hG1WT) and 14C12H1L1(hG1TM) to Fc Receptor FcγRI

The Fc receptor FcγRI, also known as CD64, can bind to the Fc fragment of IgG antibodies and is involved in antibody-dependent cell-mediated cytotoxicity (ADCC). The binding capacity of a therapeutic monoclonal antibody to Fc receptors will influence the safety and efficacy of the antibody. The affinity constants of 14C12H1L1(hG1DM), 14C12H1L1(hG4), 14C12H1L1(hG1WT) and 14C12H1L1(hG1TM) to FcγRI were measured in this experiment using a Fortebio Octet system to evaluate the ADCC activity of the antibodies. The method for determining the affinity constant of the antibodies to FcγRI by the Fortebio Octet system is briefly described as follows: the sample dilution buffer was a solution of 0.02% Tween-20 and 0.1% BSA in PBS, pH 7.4. A 1 μg/mL FcγRI solution (from Sinobio) was added to the HIS1K sensor to immobilize the FcγRI on the sensor surface for 50 s. The association and dissociation constants of the antibodies to FcγRI were both determined in the buffer with the antibody concentrations being 3.12-50 nM (serial two-fold dilution). The sensor with immobilized antigen was equilibrated in the buffer for 60 s, and then the binding of the immobilized FcγRI on the sensor to the antibodies was determined for 120 s; the dissociation of FcγRI from the antibodies was determined in 120 s. The temperature was 30° C. and the frequency was 0.3 Hz. The data were fitted and analyzed with a 1:1 model to obtain the affinity constants to FcγRI for the antibodies.

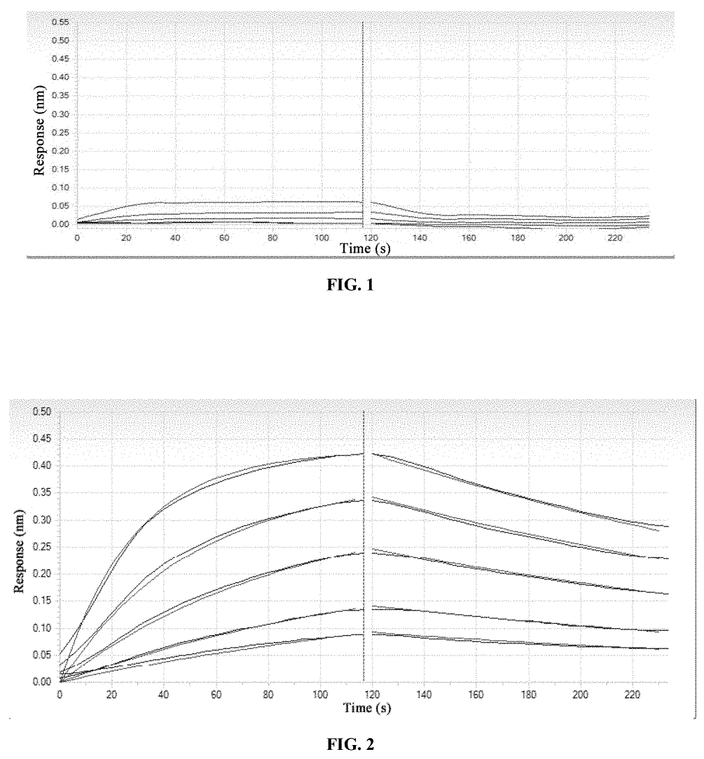

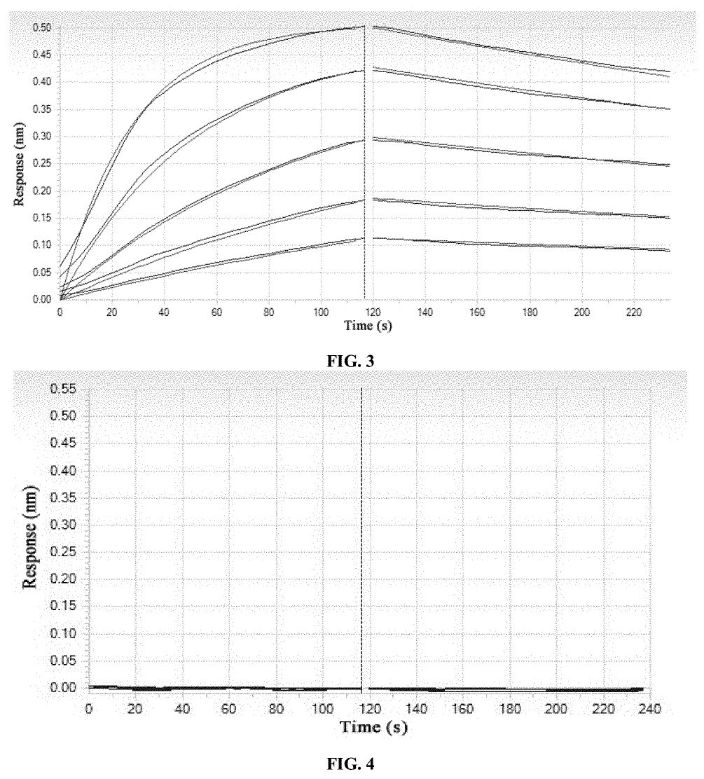

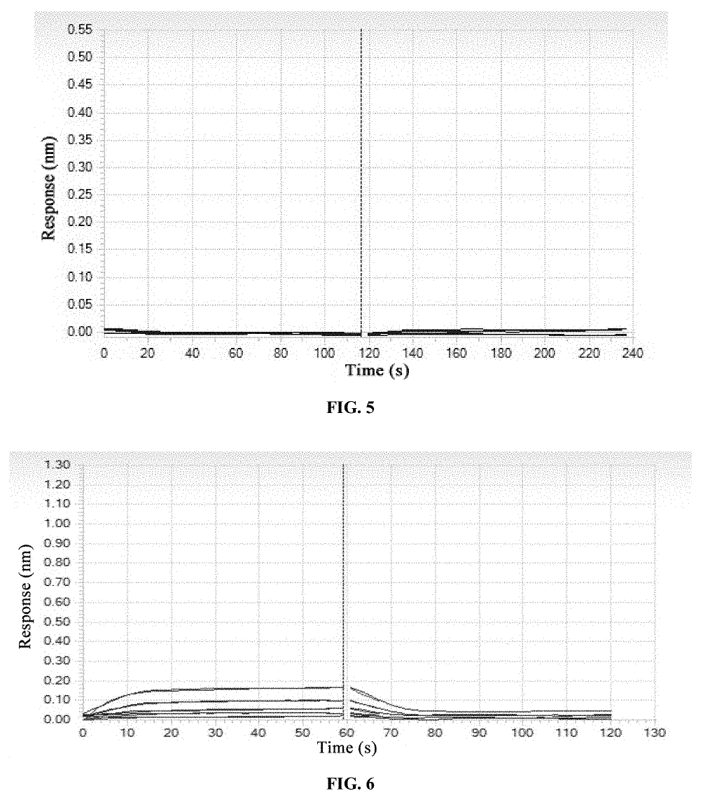

The results of affinity constant assay of 14C12H1L1(hG1DM), 14C12H1L1(hG4), 14C12H1L1(hG1WT) and 14C12H1L1(hG1TM) and the control antibody 5C10H2L2-IgG1mt to FcγRI are shown in Table 1 and FIGS. 1-5.

TABLE 1
Kinetics for binding of 14C12H1L1(hG1DM), 14C12H1L1(hG4),
14C12H1L1(hG1WT) and 14C12H1L1(hG1TM) and the control antibody
5C10H2L2-IgG1mt to FcγRI

| Antibody | K_{D} (M) | kon (1/Ms) | SE (kon) | kdis (Vs) | SE (kdis) | Rmax (nm) |
|---|---|---|---|---|---|---|
| 14C12H1L1(hG1DM) | N/A | N/A | N/A | N/A | N/A | N/A |
| 14C12H1L1(hG4) | 5.80E−09 | 6.37E+05 | 2.21E+04 | 3.69E−03 | 1.05E−04 | 0.45-0.54 |
| 14C12H1L1(hG1WT) | 2.52E−09 | 6.94E+05 | 2.19E+04 | 1.75E−03 | 9.14E−05 | 0.50-0.55 |
| 14C12H1L1(hG1TM) | N/A | N/A | N/A | N/A | N/A | N/A |
| 5C10H2L2-IgG1mt | N/A | N/A | N/A | N/A | N/A | N/A |

N/A indicates that the antibody had no binding or an extremely weak binding signal to the antigen, and thus the results were not analyzed and no corresponding data was obtained.

The results showed that both 14C12H1L1(hG4) and 14C12H1L1(hG1WT) bound to FcγRI with affinity constants of 5.80E-09 M and 2.52E-09 M, respectively; 14C12H1L1(hG1TM) and 5C10H2L2-IgG1mt had no binding or an extremely weak binding signal to FcγRI, and thus the results were not analyzed and no corresponding data was obtained.

The results suggested that the binding activities of 14C12H1L1(hG1DM) and 14C12H1L1(hG1TM) to FcγRI are effectively eliminated as compared to 14C12H1L1(hG4) and 14C12H1L1(hG1WT).

### Experimental Example 2: Affinity Assay of 14C12H1L1(hG1DM), 14C12H1L1(hG4), 14C12H1L1(hG1WT) and 14C12H1L1(hG1TM) to Fc Receptor FcγRIIIa and Subtypes Thereof

(1) Affinity Constant Assay of 14C12H1L1(hG1DM), 14C12H1L1(hG4), 14C12H1L1(hG1WT) and 14C12H1L1(hG1TM) to FcγRIIIa_V158

The Fc receptor FcγRIIIa_V158 (also known as CD16a_V158), can bind to the Fc fragment of IgG antibodies and mediate ADCC effects. The affinity constants of 14C12H1L1(hG1DM), 14C12H1L1(hG4), 14C12H1L1(hG1WT) and 14C12H1L1(hG1TM) to FcγRIIIa_V158 were measured in this experiment using a Fortebio Octet system to evaluate the ADCC activity of the antibodies.

The method for determining the affinity constant of the antibodies and the control antibody 5C10H2L2-IgG1mt to FcγRIIIa_V158 by the Fortebio Octet system is briefly described as follows: the sample dilution buffer was a solution of 0.02% Tween-20 and 0.1% BSA in PBS, pH 7.4. 5 μg/mL FcγRIIIa_V158 was immobilized on the HIS1K sensor for 120 s. The sensor was equilibrated in a buffer for 60 s, and the binding of the immobilized FcγRIIIa_V158 on the sensor to the antibodies at concentrations of 31.25-500 nM (serial two-fold dilution) was determined for 60 s. The antibody was dissociated in the buffer for 60 s. The sensor was refreshed 4 times in 10 mM glycine pH 1.5, each for 5 s. The temperature was 30° C. and the frequency was 0.3 Hz. The data were analyzed by 1:1 model fitting to obtain affinity constants.

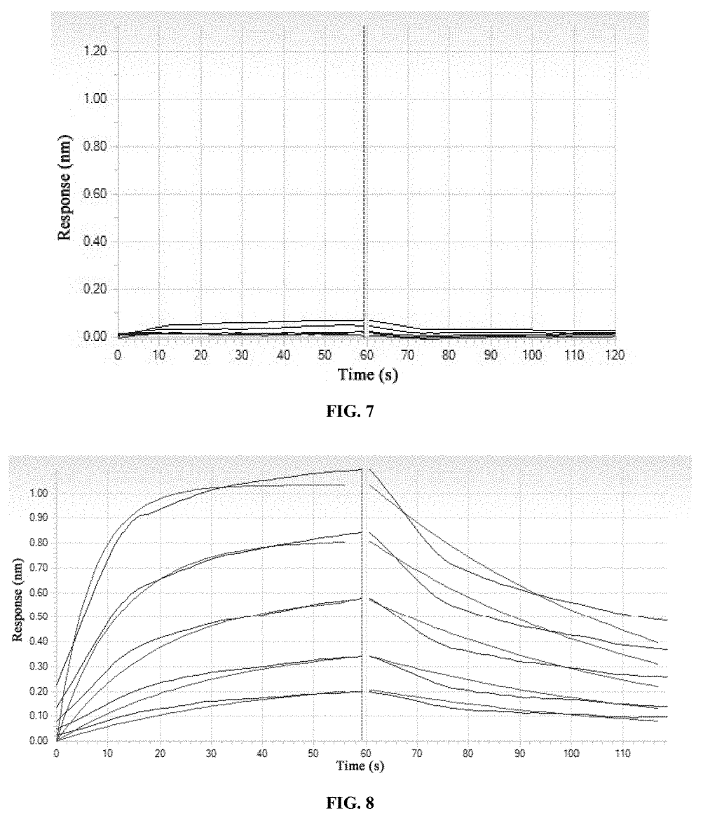

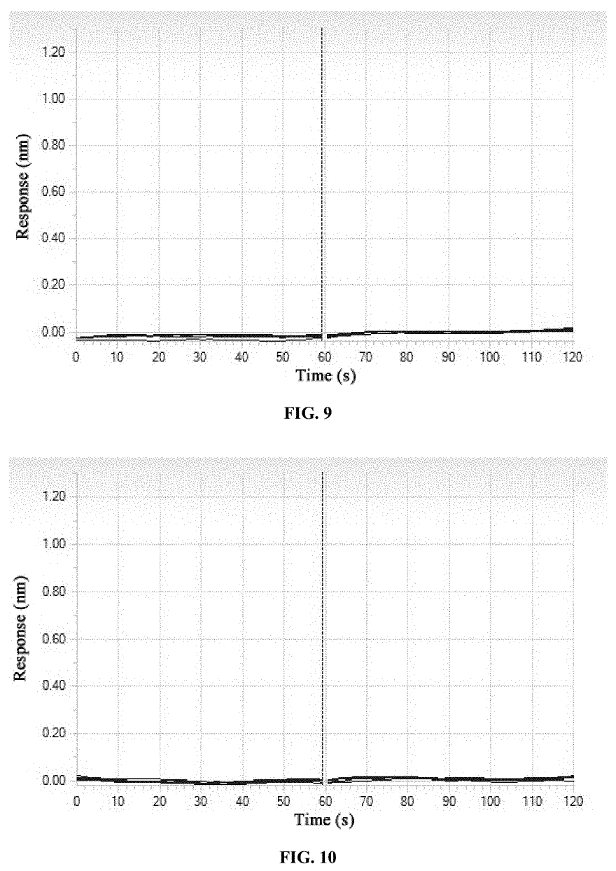

The results of affinity constant assay of 14C12H1L1(hG1DM), 14C12H1L1(hG4), 14C12H1L1(hG1WT) and 14C12H1L1(hG1TM) and the control antibody 5C10H2L2-IgG1mt to FcγRIIIa_V158 are shown in Table 2 and FIGS. 6-10.

TABLE 2
Kinetics for binding of 14C12H1L1(hG1DM), 14C12H1L1(hG4),
14C12H1L1(hG1WT) and 14C12H1L1(hG1TM) and the control antibody
5C10H2L2-IgG1mt to FcγRIIIa_V158

| Antibody | K_{D} (M) | kon (1/Ms) | SE (kon) | kdis (Vs) | SE (kdis) | Rmax (nm) |
|---|---|---|---|---|---|---|
| 14C12H1L1(hG1DM) | 6.12E−07 | 1.35E+05 | 3.74E+04 | 8.24E−02 | 8.34E−03 | 0.33-0.41 |
| 14C12H1L1(hG4) | N/A | N/A | N/A | N/A | N/A | N/A |
| 14C12H1L1(hG1WT) | 6.54E−08 | 2.61E+05 | 2.10E+04 | 1.71E−02 | 6.47E−04 | 0.80-1.17 |
| 14C12H1L1(hG1TM) | N/A | N/A | N/A | N/A | N/A | N/A |
| 5C10H2L2-IgG1mt | N/A | N/A | N/A | N/A | N/A | N/A |

N/A indicates that the antibody had no binding or an extremely weak binding signal to the antigen, and thus the results were not analyzed and no corresponding data was obtained.

The results showed that both 14C12H1L1(hG1DM) and 14C12H1L1(hG1WT) bound to FcγRIIIa_V158 with affinity constants of 6.21E-07M and 6.54E-08M, respectively; 14C12H1L1(hG4), 14C12H1L1(hG1TM) and 5C10H2L2-IgG1mt had no binding or an extremely weak binding signal to FcγRIIIa_V158, and thus the results were not analyzed.

The results suggested that the binding activities of 14C12H1L1(hG4), 14C12H1L1(hG1TM) and the control antibody 5C10H2L2-IgG1mt to FcγR IIIa_V158 are effectively eliminated as compared to 14C12H1L1(hG1DM) and 14C12H1L1(hG1WT).

(2) Affinity Constant Assay of 14C12H1L1(hG1DM), 14C12H1L1(Hg4), 14C12H1L1(Hg1Wt) and 14C12H1L1(hG1TM) to FcγRIIIa_F158

The Fc receptor FcγRIIIa_F158 (also known as CD16a_F158), can bind to the Fc fragment of IgG antibodies and mediate ADCC effects. The affinity constants of 14C12H1L1(hG1DM), 14C12H1L1(hG4), 14C12H1L1(hG1WT), 14C12H1L1(hG1TM) and the control antibody to FcγRIIIa_F158 were measured in this experiment using a Fortebio Octet system to evaluate the ADCC activity of the antibodies.

The method for determining the affinity constant of 14C12H1L1(hG1DM), 14C12H1L1(hG4), 14C12H1L1(hG1WT) and 14C12H1L1(hG1TM) to FcγRIIIa_F158 by the Fortebio Octet system is briefly described as follows: the sample dilution buffer was a solution of 0.02% Tween-20 and 0.1% BSA in PBS, pH 7.4. 5 μg/mL FcγRIIIa_F158 was immobilized on the HIS1K sensor for 120 s. The sensor was equilibrated in a buffer for 60 s, and the binding of the immobilized FcγRIIIa_F158 on the sensor to the antibodies at concentrations of 31.25-500 nM (serial two-fold dilution) was determined for 60 s. The antibody was dissociated in the buffer for 60 s. The sensor was refreshed 4 times in 10 mM glycine pH 1.5, each for 5 s. The temperature was 30° C. and the frequency was 0.3 Hz. The data were analyzed by 1:1 model fitting to obtain affinity constants.

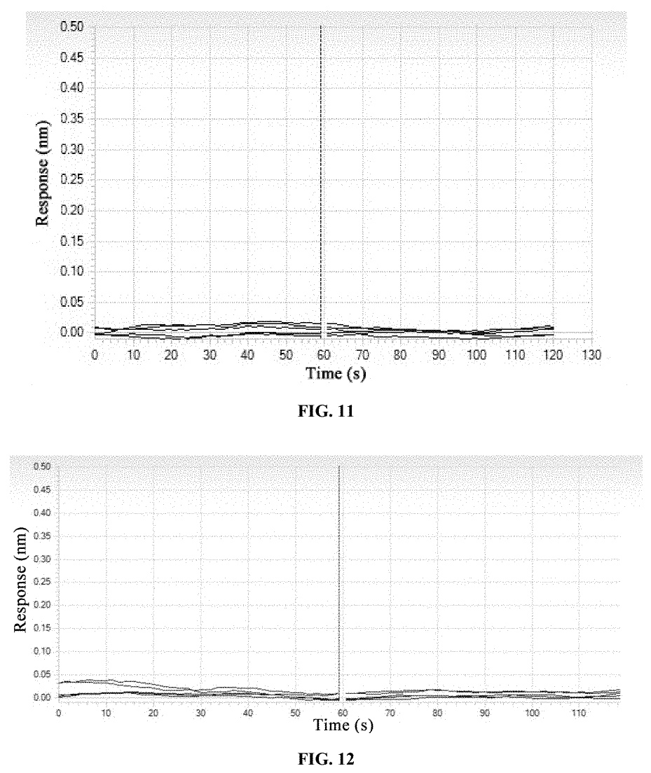

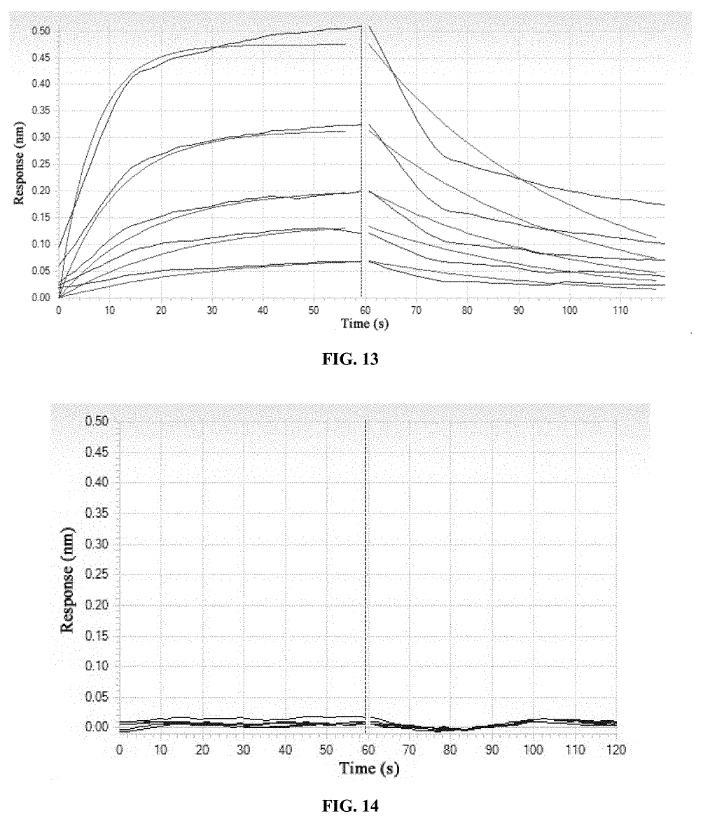

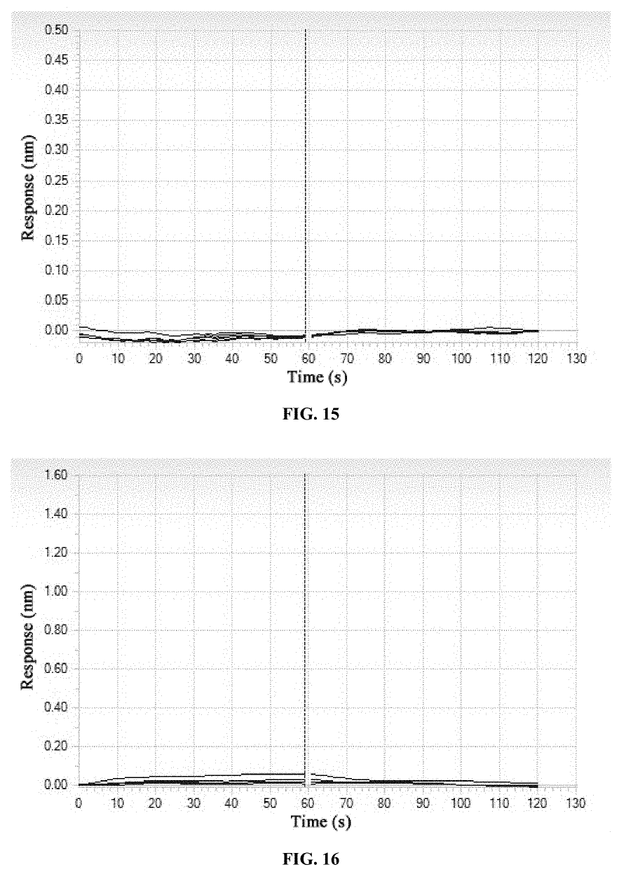

The results of affinity constant assay of 14C12H1L1(hG1DM), 14C12H1L1(hG4), 14C12H1L1(hG1WT) and 14C12H1L1(hG1TM) and the control antibody 5C10H2L2-IgG1mt to FcγRIIIa_F158 are shown in Table 3 and FIGS. 11-15.

TABLE 3
Kinetics for binding of 14C12H1L1 antibodies and isotypes thereof to FcγRIIIa_F158

| Antibody | K_{D} (M) | kon (1/Ms) | SE (kon) | kdis (Vs) | SE (kdis) | Rmax (nm) |
|---|---|---|---|---|---|---|
| 14C12H1L1(hG1DM) | N/A | N/A | N/A | N/A | N/A | N/A |
| 14C12H1L1(hG4) | N/A | N/A | N/A | N/A | N/A | N/A |
| 14C12H1L1(hG1WT) | 1.02E−07 | 2.52E+05 | 2.93E+04 | 2.56E−02 | 1.12E−03 | 0.34-0.57 |
| 14C12H1L1(hG1TM) | N/A | N/A | N/A | N/A | N/A | N/A |
| 5C10H2L2-IgG1mt | N/A | N/A | N/A | N/A | N/A | N/A |

N/A indicates that the antibody had no binding or an extremely weak binding signal to the antigen, and thus the results were not analyzed and no corresponding data was obtained.

The results showed that 14C12H1L1(hG1WT) bound to FcγRIIIa_F158 with an affinity constant of 1.02E-07M; 14C12H1L1(hG1DM), 14C12H1L1(hG4), 14C12H1L1(hG1TM) and 5C10H2L2-IgG1mt had no binding or an extremely weak binding signal to FcγRIIIa_F158, and thus the results were not analyzed and no corresponding data was obtained.

The results suggested that the binding activities of 14C12H1L1(hG1DM), 14C12H1L1(hG4) and 14C12H1L1(hG1TM) to FcγRIIIa_F158 are effectively eliminated as compared to 14C12H1L1(hG1WT).

### Experimental Example 3: Affinity Assay of 14C12H1L1(hG1DM), 14C12H1L1(hG4), 14C12H1L1(hG1WT) and 14C12H1L1(hG1TM) to Fc Receptor FcγRIIa and Subtypes Thereof

(1) Affinity Constant Assay of 14C12H1L1(hG1DM), 14C12H1L1(hG4), 14C12H1L1(hG1WT) and 14C12H1L1(hG1TM) to FcγRIIa_H131

The Fc receptor FcγRIIa_H131, also known as CD32a_H131, can bind to the Fc fragment of IgG antibodies and is involved in antibody-dependent cell-mediated cytotoxicity (ADCC). The binding capacity of a therapeutic monoclonal antibody to Fc receptors will influence the safety and efficacy of the antibody. The affinity constants of 14C12H1L1(hG1DM), 14C12H1L1(hG4), 14C12H1L1(hG1WT) and 14C12H1L1(hG1TM) to FcγRIIa_H131 were measured in this experiment using a Fortebio Octet system to evaluate the binding capacity of the antibodies to Fc receptor.

The method for determining the affinity constant of 14C12H1L1(hG1DM), 14C12H1L1(hG4), 14C12H1L1(hG1WT) and 14C12H1L1(hG1TM) to FcγRIIa_H131 by the Fortebio Octet system is briefly described as follows: the immobilization dilution buffer was a solution of PBS, 0.02% Tween-20 and 0.1% BSA, pH 7.4, and the analyte dilution buffer was a solution of 0.02% Tween-20, 0.02% casein and 0.1% BSA in PBS, pH 7.4. 5 μg/mL FcγRIIa_H131 was immobilized on the NTA sensor at an immobilization height of about 1.0 nm. The sensor was equilibrated in a buffer of 0.02% Tween-20, 0.02% casein and 0.1% BSA in PBS, pH 7.4 for 300 s of blocking, and the binding of the immobilized FcγRIIa_H131 on the sensor to the antibodies at concentrations of 12.5-200 nM (serial two-fold dilution) was determined for 60 s. The antibody was dissociated in the buffer for 60 s. The sensor was refreshed in 10 mM glycine pH 1.7 and 10 nM nickel sulfate. The temperature was 30° C. and the frequency was 0.6 Hz. The data were analyzed by 1:1 model fitting to obtain affinity constants.

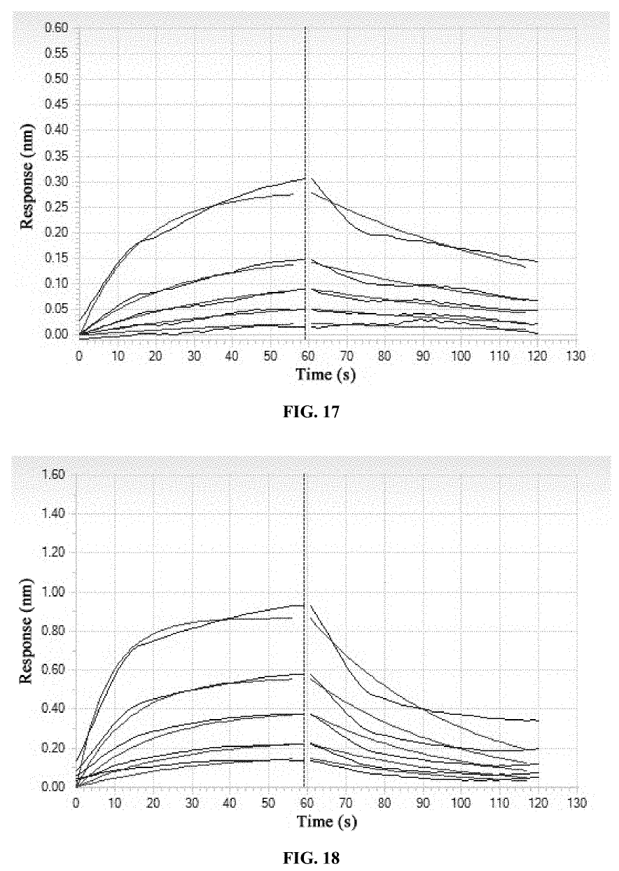

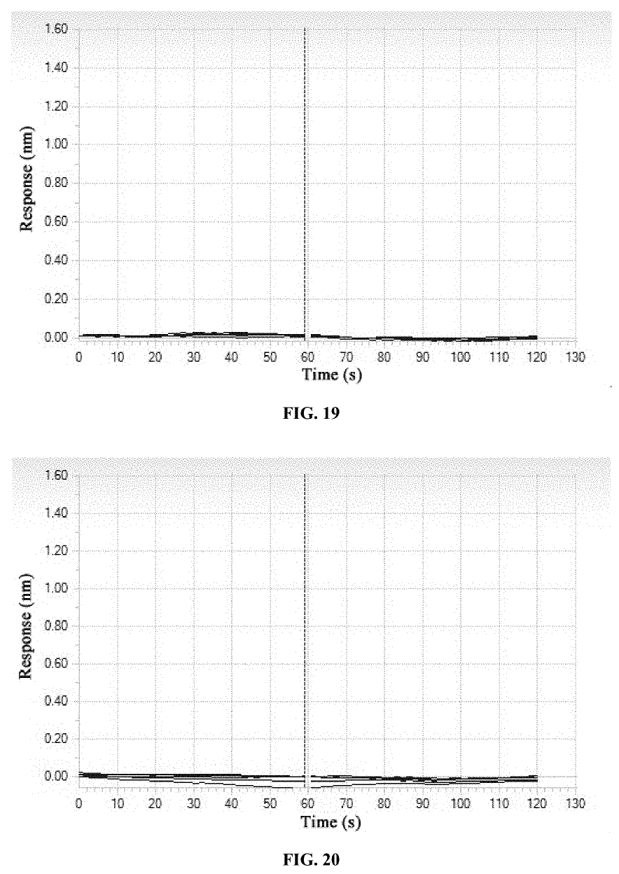

The results of affinity constant assay of 14C12H1L1(hG1DM), 14C12H1L1(hG4), 14C12H1L1(hG1WT) and 14C12H1L1(hG1TM) and the control antibody 5C10H2L2-IgG1mt to FcγRIIa_H131 are shown in Table 4 and FIGS. 16-20.

TABLE 4
Kinetics for binding of 14C12H1L1 antibodies and isotypes thereof to FcγRIIa_H131

| Sample ID | K_{D} (M) | kon (1/Ms) | SE (kon) | kdis (Vs) | SE (kdis) | Rmax (nm) |
|---|---|---|---|---|---|---|
| 14C12H1L1(hG1DM) | N/A | N/A | N/A | N/A | N/A | N/A |
| 14C12H1L1(hG4) | 5.07E−08 | 2.57E+05 | 2.37E+04 | 1.30E−02 | 6.47E−04 | 0.18-0.35 |
| 14C12H1L1(hG1WT) | 5.74E−08 | 4.65E+05 | 5.99E+04 | 2.67E−02 | 1.24E−03 | 0.82-1.12 |
| 14C12H1L1(hG1TM) | N/A | N/A | N/A | N/A | N/A | N/A |
| 5C10H2L2-IgG1mt | N/A | N/A | N/A | N/A | N/A | N/A |

N/A indicates that the antibody had no binding or an extremely weak binding signal to the antigen, and thus the results were not analyzed and no corresponding data was obtained.

The results showed that both 14C12H1L1(hG4) and 14C12H1L1(hG1WT) bound to FcγR11a_H131 with affinity constants of 5.07E-08M and 5.74E-08M, respectively; 14C12H1L1(hG1DM), 14C12H1L1(hG1TM) and 5C10H2L2-IgG1mt had no binding or an extremely weak binding signal to FcγR11a_H131, and thus the results were not analyzed and no corresponding data was obtained.

The results suggested that the binding activities of 14C12H1L1(hG1DM) and 14C12H1L1(hG1TM) to FcγR11a_H131 are effectively eliminated as compared to 14C12H1L1(hG4) and 14C12H1L1(hG1WT).

(2) Affinity Constant Assay of 14C12H1L1(hG1DM), 14C12H1L1(Hg4), 14C12H1L1(Hg1Wt) and 14C12H1L1(hG1TM) to FcγRIIa_R131

The Fc receptor FcγRIIa_R131, also known as CD32a_R131, can bind to the Fc fragment of IgG antibodies and is involved in antibody-dependent cell-mediated cytotoxicity (ADCC). The binding capacity of a therapeutic monoclonal antibody to Fc receptors will influence the safety and efficacy of the antibody. The affinity constants of 14C12H1L1(hG1DM), 14C12H1L1(hG4), 14C12H1L1(hG1WT) and 14C12H1L1(hG1TM) to FcγRIIa_R131 were measured in this experiment using a Fortebio Octet system to evaluate the binding capacity of the antibodies to Fc receptor.

The method for determining the affinity constant of 14C12H1L1(hG1DM), 14C12H1L1(hG4), 14C12H1L1(hG1WT) and 14C12H1L1(hG1TM) to FcγRIIa_R131 by the Fortebio Octet system is briefly described as follows: the immobilization dilution buffer was a solution of PBS, 0.02% Tween-20 and 0.1% BSA, pH 7.4, and the analyte dilution buffer was a solution of 0.02% Tween-20, 0.02% casein and 0.1% BSA in PBS, pH 7.4. 5 μg/mL FcγRIIa_R131 was immobilized on the NTA sensor at an immobilization height of about 1.0 nm. The sensor was equilibrated in a buffer of 0.02% Tween-20, 0.02% casein and 0.1% BSA in PBS, pH 7.4 for 300 s of blocking, and the binding of the immobilized FcγRIIa_R131 on the sensor to the antibodies at concentrations of 12.5-200 nM (serial two-fold dilution) was determined for 60 s. The antibody was dissociated in the buffer for 60 s. The sensor was refreshed in 10 mM glycine pH 1.7 and 10 nM nickel sulfate. The temperature was 30° C. and the frequency was 0.6 Hz. The data were analyzed by 1:1 model fitting to obtain affinity constants.

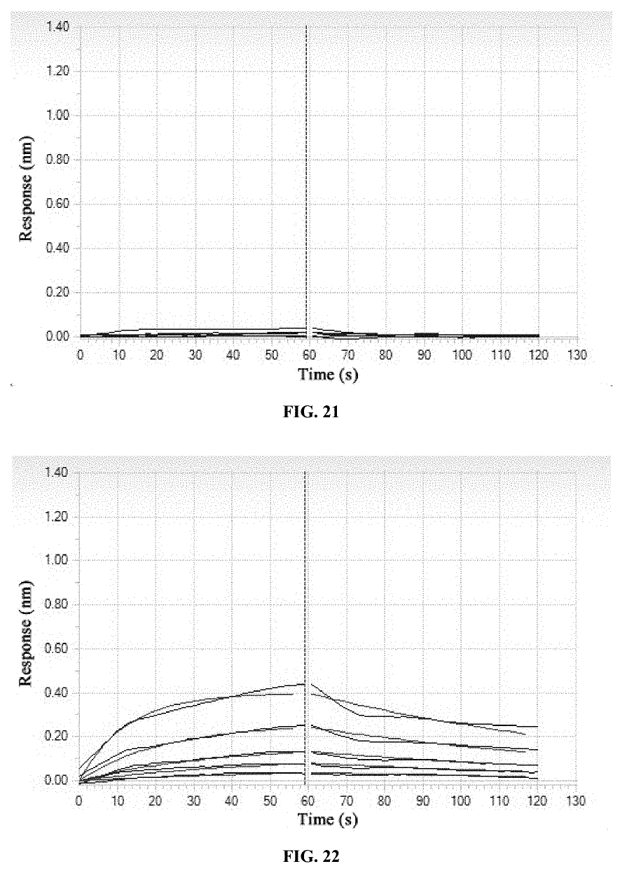

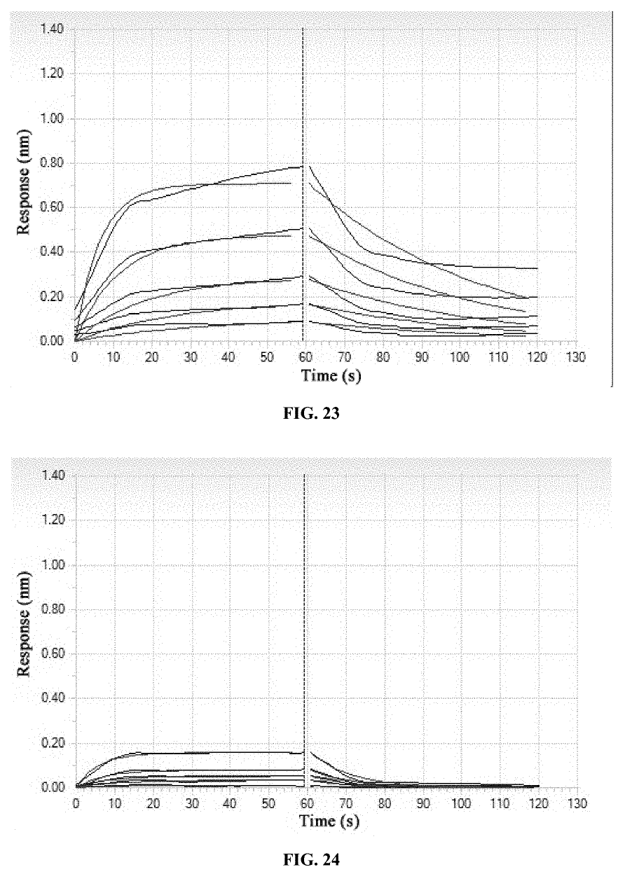

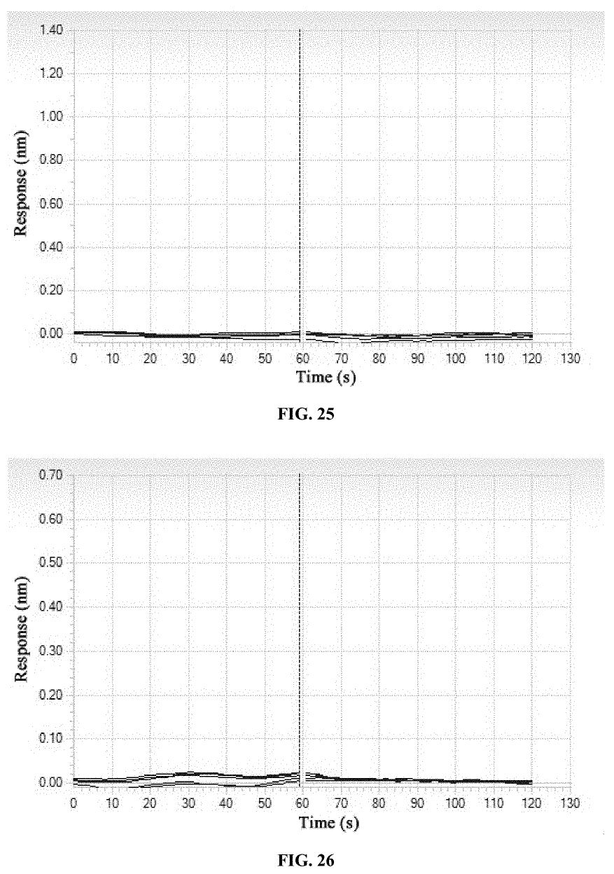

The results of affinity constant assay of 14C12H1L1(hG1DM), 14C12H1L1(hG4), 14C12H1L1(hG1WT) and 14C12H1L1(hG1TM) and the control antibody 5C10H2L2-IgG1mt to FcγRIIa_R131 are shown in Table 5 and FIGS. 21-25.

TABLE 5
Kinetics for binding of 14C12H1L1 antibodies and isotypes thereof to FcγRIIa_R131

| Antibody | K_{D} (M) | kon (1/Ms) | SE (kon) | kdis (Vs) | SE (kdis) | Rmax (nm) |
|---|---|---|---|---|---|---|
| 14C12H1L1(hG1DM) | N/A | N/A | N/A | N/A | N/A | N/A |
| 14C12H1L1(hG4) | 3.13E−08 | 3.50E+05 | 3.05E+04 | 1.10E−02 | 6.43E−04 | 0.21-0.46 |
| 14C12H1L1(hG1WT) | 3.46E−08 | 6.60E+05 | 9.46E+04 | 2.28E−02 | 1.33E−03 | 0.39-0.83 |
| 14C12H1L1(hG1TM) | 2.32E−07 | 4.04E+05 | 9.45E+04 | 9.38E−02 | 4.89E−03 | 0.19-0.35 |
| 5C10H2L2-IgG1mt | N/A | N/A | N/A | N/A | N/A | N/A |

N/A indicates that the antibody had no binding or an extremely weak binding signal to the antigen, and thus the results were not analyzed and no corresponding data was obtained.

The results showed that 14C12H1L1(hG4), 14C12H1L1(hG1WT) and 14C12H1L1(hG1TM) bound to FcγRIIa_R131 with affinity constants of 3.13E-08M, 3.46E-08M and 2.32E-07M, respectively; 14C12H1L1(hG1DM) and 5C10H2L2-IgG1mt had no binding or an extremely weak binding signal to FcγRIIa_R131, and thus the results were not analyzed and no corresponding data was obtained.

The results suggest that among the antibodies with binding activities, 14C12H1L1(hG1TM) has the weakest binding capacity and the lowest binding activity to FcγRIIa_R131 as compared to 14C12H1L1(hG4) and 14C12H1L1(hG1WT).

### Experimental Example 4: Affinity Constant Assay of 14C12H1L1(hG1DM), 14C12H1L1(hG4), 14C12H1L1(hG1WT) and 14C12H1L1(hG1TM) to FcγRIIb

The Fc receptor FcγRIIb (also known as CD32b), can bind to the Fc fragment of IgG antibodies, down-regulate functions of immune cells, inhibit the activation and proliferation of immune cells and inhibit the secretion of cytokines. The affinity constants of the antibodies to FcγRIIb were measured in this experiment using a Fortebio Octet system to evaluate the binding capacity of 14C12H1L1(hG1DM), 14C12H1L1(hG4), 14C12H1L1(hG1WT) and 14C12H1L1(hG1TM) to Fc receptor.

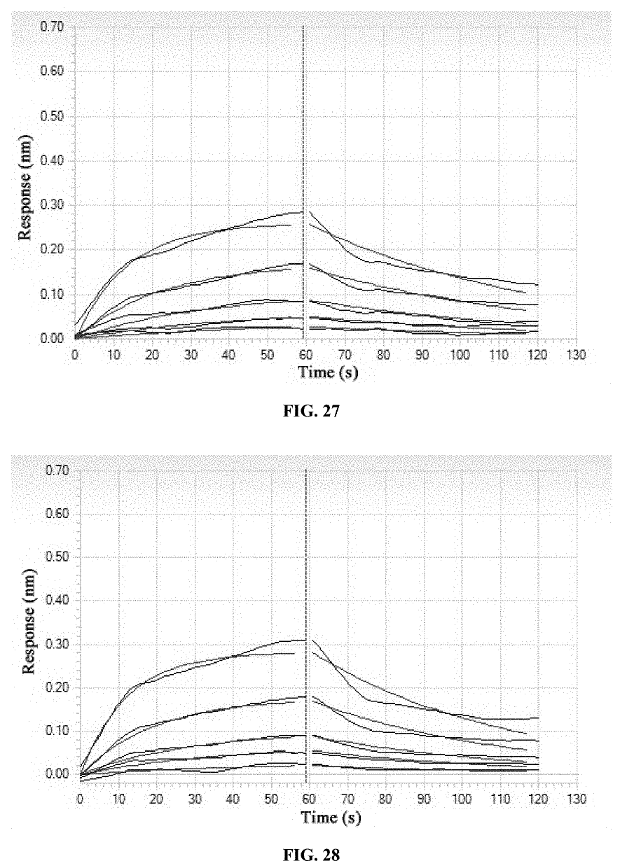

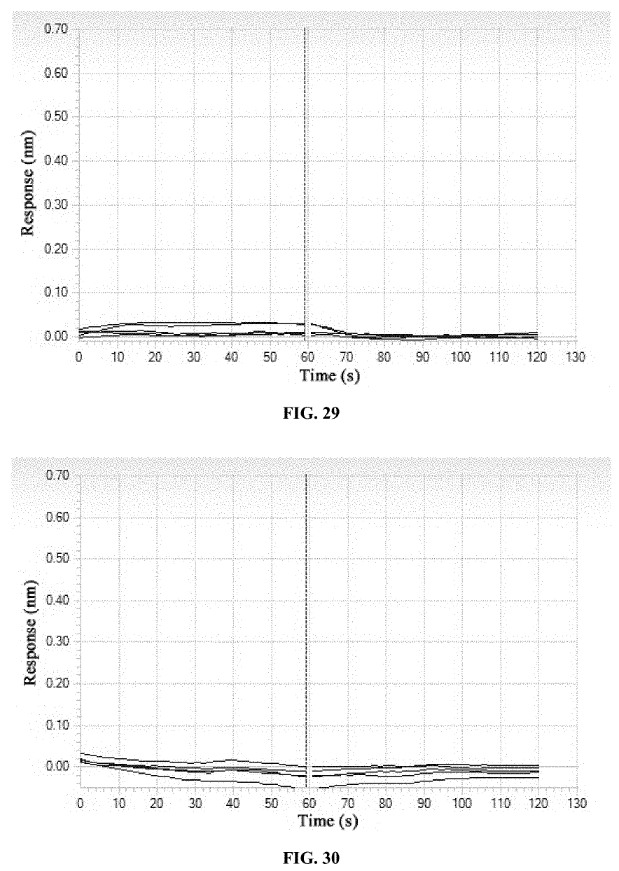

The method for determining the affinity constant of 14C12H1L1(hG1DM), 14C12H1L1(hG4), 14C12H1L1(hG1WT) and 14C12H1L1(hG1TM) to FcγRIIb by the Fortebio Octet system is briefly described as follows: the immobilization dilution buffer was a solution of PBS, 0.02% Tween-20 and 0.1% BSA, pH 7.4, and the analyte dilution buffer was a solution of 0.02% Tween-20, 0.02% casein and 0.1% BSA in PBS, pH 7.4. 5 μg/mL hFcγRIIb-his was immobilized on the NTA sensor at an immobilization height of about 1.0 nm. The sensor was equilibrated in a buffer of 0.02% Tween-20, 0.02% casein and 0.1% BSA in PBS, pH 7.4 for 300 s of blocking, and the binding of the immobilized hFcγRIIb-his on the sensor to the antibodies at concentrations of 12.5-200 nM (serial two-fold dilution) was determined for 60 s. The antibody was dissociated in the buffer for 60 s. The sensor was refreshed in 10 mM glycine pH 1.7 and 10 nM nickel sulfate. The temperature was 30° C. and the frequency was 0.6 Hz. The data were analyzed by 1:1 model fitting to obtain affinity constants. The results of affinity constant assay of 14C12H1L1(hG1DM), 14C12H1L1(hG4), 14C12H1L1(hG1WT) and 14C12H1L1(hG1TM) and the control antibody 5C10H2L2-IgG1mt to FcγRIIb are shown in Table 6 and FIGS. 26-30.

TABLE 6
Kinetics for binding of 14C12H1L1 antibodies and isotypes thereof to FcγRIIb

| Antibody | K_{D} (M) | kon (1/Ms) | SE (kon) | kdis (Vs) | SE (kdis) | Rmax (nm) |
|---|---|---|---|---|---|---|
| 14C12H1L1(hG1DM) | N/A | N/A | N/A | N/A | N/A | N/A |
| 14C12H1L1(hG4) | 5.62E−08 | 2.88E+05 | 2.94E+04 | 1.62E−02 | 7.63E−04 | 0.22-0.33 |
| 14C12H1L1(hG1WT) | 6.13E−08 | 3.18E+05 | 3.48E+04 | 1.95E−02 | 8.96E−04 | 0.16-0.37 |
| 14C12H1L1(hG1TM) | N/A | N/A | N/A | N/A | N/A | N/A |
| 5C10H2L2-IgG1mt | N/A | N/A | N/A | N/A | N/A | N/A |

N/A indicates that the antibody had no binding or an extremely weak binding signal to the antigen, and thus the results were not analyzed and no corresponding data was obtained.

The results showed that both 14C12H1L1(hG4) and 14C12H1L1(hG1WT) bound to FcγRIIb with affinity constants of 5.62E-08M and 6.13E-08M, respectively; 14C12H1L1(hG1DM), 14C12H1L1(hG1TM) and 5C10H2L2-IgG1mt had no binding or an extremely weak binding signal to FcγRIIb, and thus the results were not analyzed and no corresponding data was obtained.

The results suggested that the binding activities of 14C12H1L1(hG1DM) and 14C12H1L1(hG1TM) to FcγRIIb are effectively eliminated as compared to 14C12H1L1(hG4) and 14C12H1L1(hG1WT).

### Experimental Example 5: Affinity Assay of 14C12H1L1(hG1DM), 14C12H1L1(hG4), 14C12H1L1(hG1WT) and 14C12H1L1(hG1TM) to C1q

Serum complement C1q can bind to the Fc fragment of IgG antibodies and mediate CDC effects. The binding capacity of a therapeutic monoclonal antibody to C1q will influence the safety and efficacy of the antibody. The affinity constants of 14C12H1L1(hG1DM), 14C12H1L1(hG4), 14C12H1L1(hG1WT) and 14C12H1L1(hG1TM) to C1q were measured in this experiment using a Fortebio Octet system to evaluate the CDC activity of the antibodies.

The method for determining the affinity constants of the antibodies to C1q by the Fortebio Octet system is briefly described as follows: the sample dilution buffer was a solution of 0.02% Tween-20 and 0.1% BSA in PBS, pH 7.4. 50 μg/mL antibody was immobilized on the FAB2G sensor at an immobilization height of about 2.0 nm. The sensor was equilibrated in a buffer for 60 s for blocking, and the binding of the immobilized antibody on the sensor to the antigen C1q at concentrations of 1.25-20 nM (serial two-fold dilution) was determined for 60 s. The antigen and antibody were dissociated in the buffer for 60 s. The sensor was refreshed 4 times in 10 mM glycine pH 1.7, each for 5 s. The shaking speed of the sample plate was 1000 rpm, the temperature was 30° C. and the frequency was 0.6 Hz. The data were analyzed by 1:1 model fitting to obtain affinity constants. The data acquisition software was Fortebio Data Acquisition 7.0, and the data analysis software was Fortebio Data Analysis 7.0.

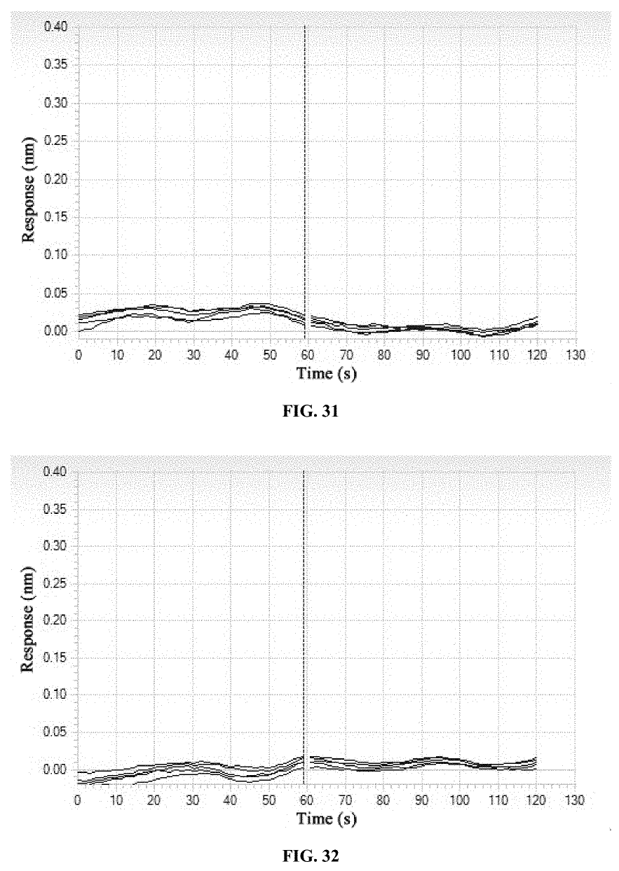

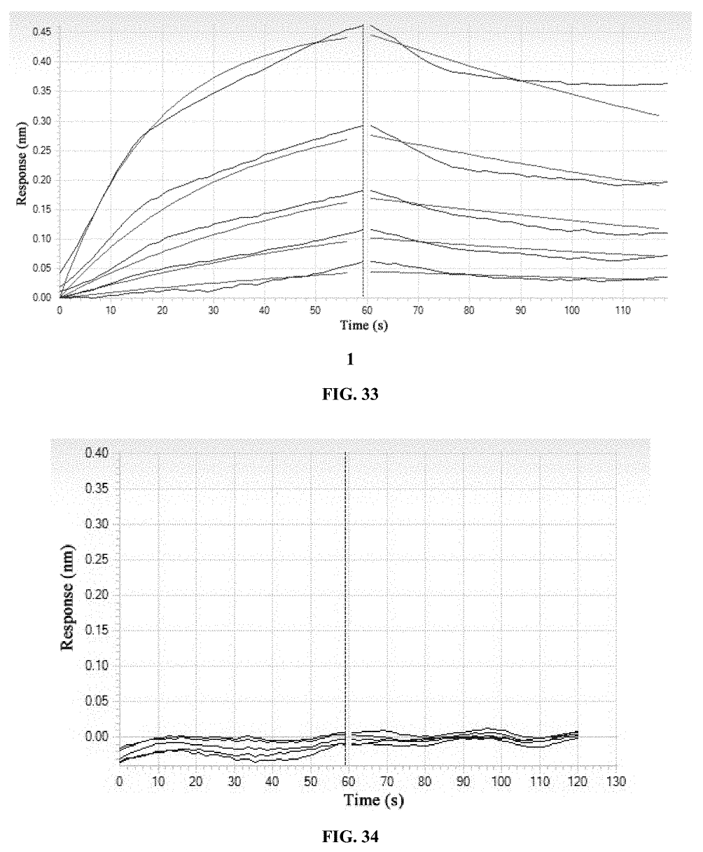

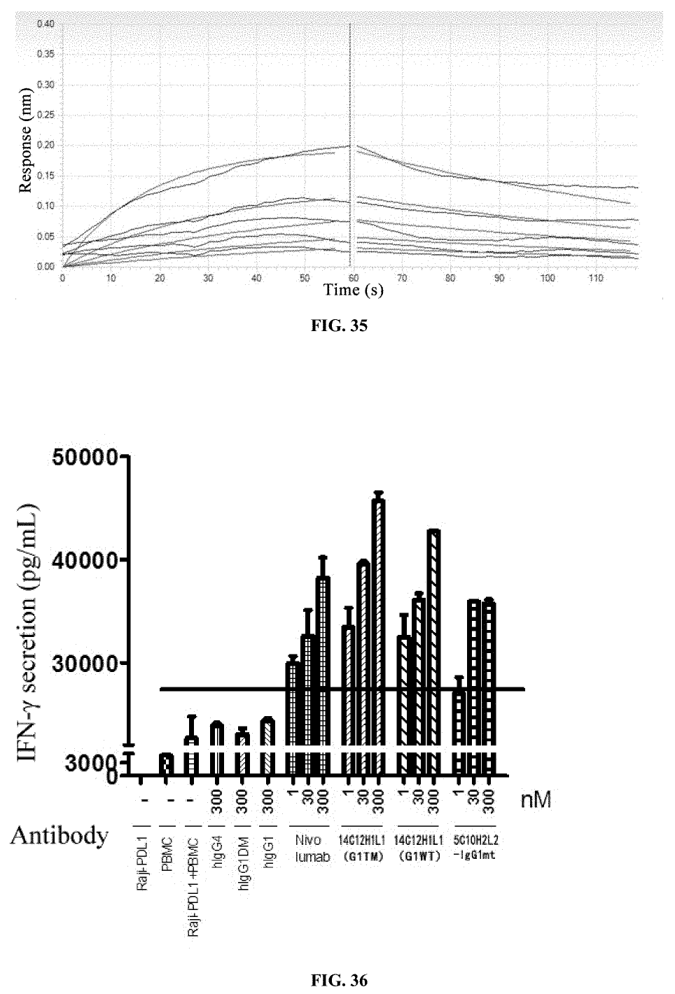

The results of affinity constant assay of 14C12H1L1(hG1DM), 14C12H1L1(hG4), 14C12H1L1(hG1WT) and 14C12H1L1(hG1TM) and the control 5C10H2L2-IgG1mt to C1q are shown in Table 7 and FIGS. 31-35.

TABLE 7
Kinetics for binding of 14C12H1L1 antibodies and isotypes thereof to C1q

| Antibody | K_{D} (M) | kon (1/Ms) | SE (kon) | kdis (Vs) | SE (kdis) | Rmax (nm) |
|---|---|---|---|---|---|---|
| 14C12H1L1(hG1DM) | N/A | N/A | N/A | N/A | N/A | N/A |
| 14C12H1L1(hG4) | N/A | N/A | N/A | N/A | N/A | N/A |
| 14C12H1L1(hG1WT) | 1.35E−09 | 4.83E+06 | 4.24E+05 | 6.54E−03 | 5.33E−04 | 0.32-0.53 |
| 14C12H1L1(hG1TM) | N/A | N/A | N/A | N/A | N/A | N/A |
| 5C10H2L2-IgG1mt | 4.43E−09 | 2.38E+06 | 4.21E+05 | 1.05E−02 | 1.10E−03 | 0.19-0.26 |

N/A indicates that the antibody had no binding or an extremely weak binding signal to the antigen, and thus the results were not analyzed and no corresponding data was obtained.

The results showed that 14C12H1L1(hG1WT) bound to C1q with an affinity constant of 1.35E-09M; 14C12H1L1(hG1DM), 14C12H1L1(hG4) and 14C12H1L1(hG1TM) had no binding or an extremely weak binding signal to C1q, and thus the results were not analyzed and no corresponding data was obtained.

The results also showed that 5C10H2L2-IgG1mt bound to C1q with an affinity constant of 4.43E-09, indicating that it has binding activity to C1q and can cause CDC effects.

### Experimental Example 6: Pharmacodynamic Activities of 14C12H1L1(hG1WT) and 14C12H1L1(hG1TM) in Co-Culture System of Peripheral Blood Mononuclear Cells and Raji-PDL1 Cells

In this experiment, the pharmacodynamic activity of anti-PD-1 antibodies 14C12H1L1 (hG1WT) and 14C12H1L1 (hG1TM), and the control anti-PD-L1 antibodies 5C10H2L2-IgG1mt and nivolumab in relieving immunosuppression mediated by PD-1/PD-L1 were detected in a co-culture system of peripheral blood mononuclear cells and Raji-PDL1 cells. In the mixed lymphocyte reaction, when isolated peripheral blood mononuclear cells (containing immune cells expressing immunocompetent PD-1) and the Raji-PDL1 cells expressing PD-L1 are co-cultured, the interaction of PD-1 and PD-L1 can mediate the function inhibition of the immune cells, showing reduced secretion of cytokines IFN-γ and IL-2. Anti-PD-1 or anti-PD-L1 antibodies can relieve such immunosuppression of immune cells, leading to increased cytokine secretion. Raji is a B cell line. As described above, B cells can be used as antigen presenting cells to mediate the immune response of immune cells to tumor cells. In the present invention, the Raji-PDL1 and PBMCs co-culture system was used to evaluate the pharmacological activity of the anti-PD-1 antibodies, and a Raji-PDL1, PBMCs and tumor cell co-culture system was used to evaluate the pharmacological activity of the anti-PD-1 antibodies in different tumors.

Peripheral blood mononuclear cells were isolated by the Ficoll-Paque Plus (GE Healthcare, Cat No.: 171440-02), and then stimulated with SEB (0.5 μg/mL) for two days. The stimulated mature peripheral blood mononuclear cells (1×10⁵ cells/well) and Raji-PDL1 cells (1×10⁵ cells/well) treated with MMC (Mito-mycin C with a treatment concentration of 2 μg/mL) for 1 h were added to a 96-well plate before 14C12H1L1(hG1WT), 14C12H1L1(hG1TM), the control antibody nivolumab or the control anti-PD-L1 antibody 5C10H2L2-IgG1mt was added. The mixture was well mixed and incubated. After 3 days, the culture supernatant was collected and detected for IFN-γ secretion and IL-2 secretion by an ELISA kit (purchased from Dakewe Biotech Co., Ltd.).

The results of the IFN-γ secretion in the mixed lymphocyte reaction are shown in FIG. 36. The results showed that in the co-culture system of PBMCs and Raji-PDL1, 14C12H1L1(hG1TM) induced a significantly higher IFN-γ secretion than those induced by 14C12H1L1(hG1WT), nivolumab or 5C10H2L2-IgG1mt at the same dose level.

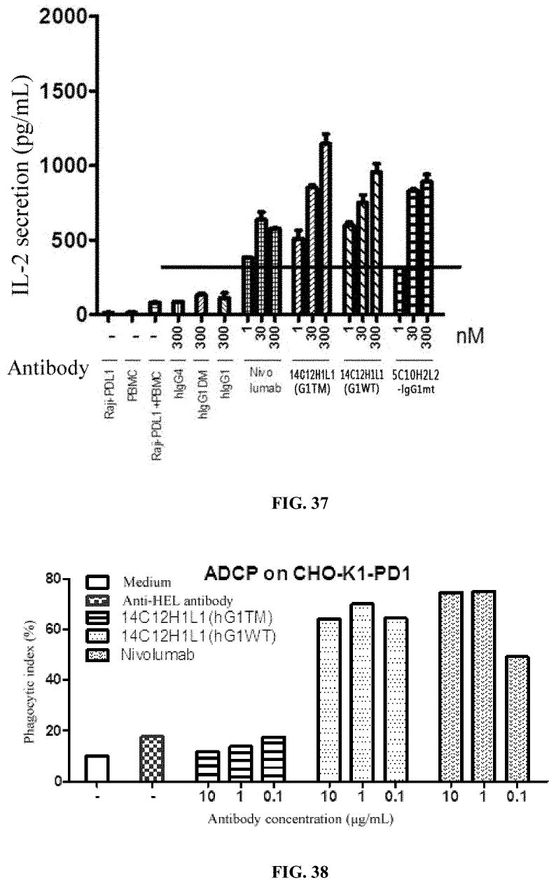

The results of the IL-2 secretion in mixed lymphocyte reaction are shown in FIG. 37. The results showed that in the co-culture system of PBMCs and Raji-PDL1, 14C12H1L1(hG1TM) induced a significantly higher IL-2 secretion than those induced by 14C12H1L1(hG1WT), nivolumab or 5C10H2L2-IgG1mt at the same dose level.

The results suggested that the pharmacodynamic activity of 14C12H1L1(hG1TM) in relieving the immunosuppression mediated by PD-1/PD-L1 is significantly superior to that of nivolumab, 14C12H1L1(hG1WT) or 5C10H2L2-IgG1mt.

### Experimental Example 7: Antibody-Mediated Phagocytic Activities of Nivolumab, 14C12H1L1(hG1WT) and 14C12H1L1(hG1TM) on CHO-K1-PD1

To detect the antibody dependent cellular phagoxytosis (ADCP) activity, murine macrophages were used as effector cells and cell lines overexpressing PD1 were used as target cells. The femoral bone marrow of Blab/c mice (purchased from Guangdong Medical Laboratory Animal Center) was first aseptically collected and lysed by erythrocyte lysis buffer on ice for 5 min. The lysis was terminated with DMEM complete medium (containing 10% FBS), and the lysate was centrifuged at 1000 rpm and washed twice. The cell pellet was resuspended in 10 mL of DMEM complete medium and M-CSF were added at a working concentration of 100 ng/mL. The cells were cultured for 7 days at 37° C. and 5% CO₂ in a cell culture chamber for induction. Half of the medium was exchanged and M-CSF was added on Days 3 and 5. The induction of cells was completed on day 7. The cells were digested with 0.05% trypsin. Macrophages were collected, and centrifuged at 750×g for 5 min. The supernatant was discarded and the cells were suspended in DMEM complete medium (containing 10% FBS) and counted. The cell was adjusted to a proper density and filled into sterile EP tubes for further use. CHO-K1-PD1 cells (a CHO-K1 cell line overexpressing PD1) were centrifuged at 170×g for 5 min, washed once with PBS, resuspended and counted. The viability was determined. Carboxyfluorescein diacetate succinimidyl ester (CFSE) was diluted to 2.5 μM with PBS to resuspend the cells (staining density: 10 million cells/mL). A proper amount of the cells were incubated in a cell incubator for 20 min. 6 mL of DMEM complete medium was added to stop the staining. The cells were centrifuged at 170×g for 5 min, and the supernatant was discarded. 1 mL of DMEM complete medium was added. The cells were incubated in an incubator for 10 min, and adjusted to the experiment density. The cells were coded as CHO-K-PD1-CFSE.

The test antibodies were diluted in DMEM complete medium to 20, 2 and 0.2 μg/mL (the working concentrations were 10, 1 and 0.1 μg/mL). An anti-HEL IgG1 antibody and a medium were used as the isotype control group and a blank control group. According to the study design, the diluted antibodies and CHO-K1-PD1-CFSE cells were added into 1.5-mL EP tubes containing macrophages (the final volume was 100 μL and the effector-to-target ratio was 50,000:150,000). The mixture was well mixed for resuspension and incubated in an incubator at 37° C. for 2 h. 800 μL of PBS containing 1% bovine serum albumin (BSA) was added at room temperature to each tube. The mixture was centrifuged at 500×g for 5 min, and the supernatant was discarded. The cells were washed once with 800 μL of 1% PBSA. APC anti-mouse/human CD11b antibody (Biolegend, Cat. No.: 101212) was diluted 400-fold with PBSA and added to the corresponding samples at 100 μL/sample. The mixture was mixed well, incubated on ice for 40 min, and washed with 800 μL of 1% PBSA and centrifuged at 1200×g for 5 min twice, and the supernatant was discarded. 200 μL of 1% PBSA was added to each tube to resuspend the cells. The cells were transferred to loading tube and analyzed by BD FACS Calibur flow cytometer. Macrophages in the system were APC⁺ positive, and macrophages involved in phagocytosis were APC and CFSE double positive. The phagocytosis rate was determined as the ratio of the number of double positive cells to the number of APC positive cells, and the antibody-mediated ADCP activity was evaluated. The ADCP activity of each group, represented by P %, was calculated according to the following formulas:

$$P \% = \frac{Number of macrophages involved in phagocytosis}{Total number of macrophages} \times 100 \%$$

The results are shown in FIG. 38.

The results showed that at the same concentration, the phagocytic rates of 14C12H1L1(hG1WT) and nivolumab were 3.94 and 4.26 times that of the isotype control anti-HEL antibody, respectively, indicating that 14C12H1L1 (hG1WT) and nivolumab have ADCP effects; and at the same concentration, the phagocytic rate of 14C12H1L1(hG1TM) was comparable to that of the isotype control antibody, indicating that 14C12H1L1(hG1TM) has no ADCP effect.

The results suggest that the amino acid mutation introduced by 14C12H1L1(hG1TM) can effectively eliminate the ADCP effect, and a surprising technical effect is obtained.

### Experimental Example 8: Pharmacodynamic Evaluation of 14C12H1L1(hG1Tm)+Anlotinib Hydrochloride in Scid/Beige Immunodeficient Mouse Model Bearing Human Non-Small Cell Lung Cancer HCC827 Subcutaneous Xenograft Tumor

Female Scid/beige immunodeficient mice (purchased from Vital River) were divided into groups of 8. 0.2 μg/mL Staphylococcus aureus enterotoxin B (SEB) was added to 1 million/mL PBMC suspension. PBMC s were incubated for 3 days for activation to increase the expression of PD1 on PBMCs. Mice were grafted subcutaneously with a mixture of 800,000 SEB-activated PBMCs and 6,000,000 HCC827 human non-small cell lung cancer cells (purchased from GuangZhou Jennio Biotech Co., Ltd.) on day 0, and divided into 2 groups, the isotype control antibody group (i.e., the anti-HEL antibody, prepared by Zhongshan Akeso Biopharma as described above) and the 14C12H1L1(hG1TM)+anlotinib hydrochloride group. The 14C12H1L1(hG1TM) was administered through the tail vein once weekly (the first dose was co-administered subcutaneously with the cells), and anlotinib was administered by oral gavage once daily for 30 days. The specific protocol is shown in Table 8. Tumors were measured continuously in the experiment, and the volume was calculated according to the formula: a (tumor length)×b(tumor width)×b(tumor width)/2.

TABLE 8
Experimental planning and grouping

| Group | n | Model | Dosage information |
|---|---|---|---|
| Isotype control | 7 | 6,000,000 | Anti-HEL antibody, 10 mg/kg, |
| | | HCC827 cells | administered via tail vein once weekly |
| | | +800,000 | for 9 doses (the first dose was co- |
| | | PBMCs (SEB- | administered subcutaneously with the |
| | | activated for 3 | cells) |
| 14C12H1L1 | 8 | days) | 14C12H1L1(hG1TM), 10 mg/kg, |
| (hG1TM) | | Subcutaneous | administered via tail vein once weekly |
| + anlotinib | | xenograft (d0) | for 9 doses (the first dose was co- |
| | | | administered subcutaneously with the |
| | | | cells) |
| | | | Anlotinib hydrochloride, 3 mg/kg, |
| | | | administered by oral gavage daily for 30 |
| | | | days |

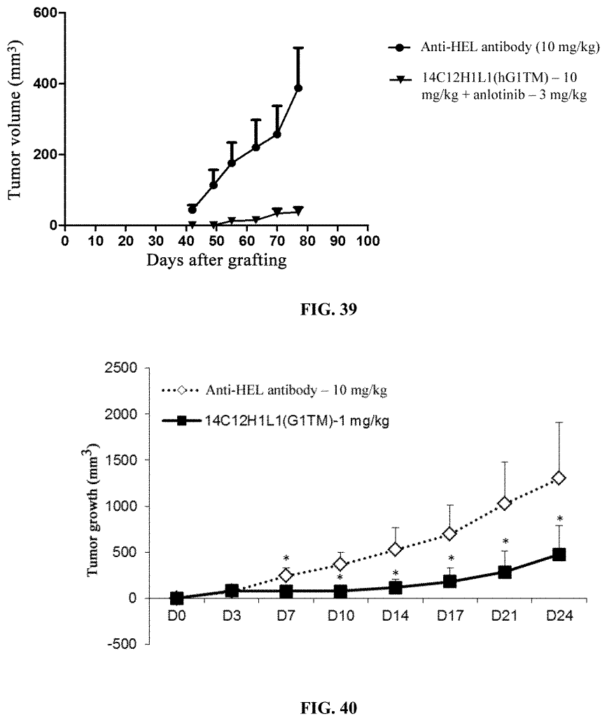

The experimental results are shown in FIG. 39.

The results showed that 14C12H1L1(hG1TM)+anlotinib hydrochloride significantly inhibited the increase in tumor volume of human non-small cell lung cancer cells, indicating good tumor killing effects.

### Experimental Example 9: Pharmacodynamic Evaluation of 14C12H1L1(hG1TM) in C57BL/6-hPD1/hPDL1/hCD73 Mouse Model Bearing Colon Cancer MC38-hPDL1/hCD73 Subcutaneous Graft Tumor

The mouse MC38 cell line is a mouse colorectal cancer cell line. It has been demonstrated that the MC38 cells line is a useful model for studying human MSI-H/dMMR tumors (Efremova M et al., Nat Commun., 2018; 9(1):32).

Female C57BL/6-hPD1/hPDL1/hCD73 mice (purchased from Nanjing GemPharmatech Co., Ltd.) were divided into groups of 8 and grafted subcutaneously on the right forelimb with colon cancer MC38-hPDL1/hCD73 cells (purchased from Nanjing GemPharmatech Co., Ltd.) (2×10⁶ cells/100 μL/mouse). The day of grafting was defined as D0. The dosing volume was adjusted according to the body weight: 10 μL/g mouse body weight (g). The anti-HEL antibody (the preparation and the source are the same as those of the Experimental Example 8) or 14C12H1L1(hG1TM) was administrated intraperitoneally twice weekly for 3 weeks, in a total of 6 doses. The specific protocol is shown in Table 9. Tumors were measured continuously in the experiment, and the volume was calculated as the following formula:

tumor volume (mm³)=(tumor length×(tumor width)²)/2.

TABLE 9
Protocol and grouping

| Group | N | Dose (mg/kg) | Frequency | Cycle |
|---|---|---|---|---|
| Isotype Control | 8 | 10 | BIW*3 | 6 doses in |
| | | | | total |
| 14C12H1L1 | 8 | 1 | BIW*3 | 6 doses in |
| (hG1TM) | | | | total |

The experimental results are shown in FIG. 40.

The results showed that as compared to the isotype control antibody, the tumor growth was inhibited, indicating that 14C12H1L1(hG1TM) can significantly inhibit the proliferation of MC38 cells, and can effectively treat solid tumors of the MSI-H/dMMR phenotype, such as colon and/or rectal cancers.

### Experimental Example 10: Effectively Enhanced Immune Response of Immune Cells to Human Gastric Cancer KATO III Cells by 14C12H1L1(hG1TM)

PBMCs were isolated from healthy human peripheral blood according to the Ficoll-Paque™ Plus reagent instruction, and the isolated PBMCs were counted and frozen. Raji-PDL1 cells were cultured in RPMI 1640+10% FBS complete medium, and KATO III cells (purchased from Chinese Academy of Sciences Shanghai Cell Bank) were cultured in DMEM+10% FBS complete medium. PBMCs were thawed and activated with 0.5 μg/mL SEB for two days. On the day of the experiment, Raji-PDL1 cells were treated with 2 μg/mL MMC for 1 h. SEB-activated PBMCs and MMC-treated Raji-PDL1 cells were collected, washed twice with PBS, resuspended in RPMI 1640+10% FBS complete medium and counted. Raji-PDL1 and PBMC cells were seeded on 96-well plates at 1×10⁵ cells/well. KATO III cells in logarithmic growth phase were collected and seeded on the 96-well plate at 5×10⁴ cells/well. The diluted antibody was added according to the study design. The mixture was mixed evenly and incubated in a 5% CO₂ incubator at 37° C. for 3 days. After 3 days, the cell culture supernatant was collected and tested for IL-2 according to ELISA KIT instruction. The media in this experiment were all 10% FBS+RPMI 1640.

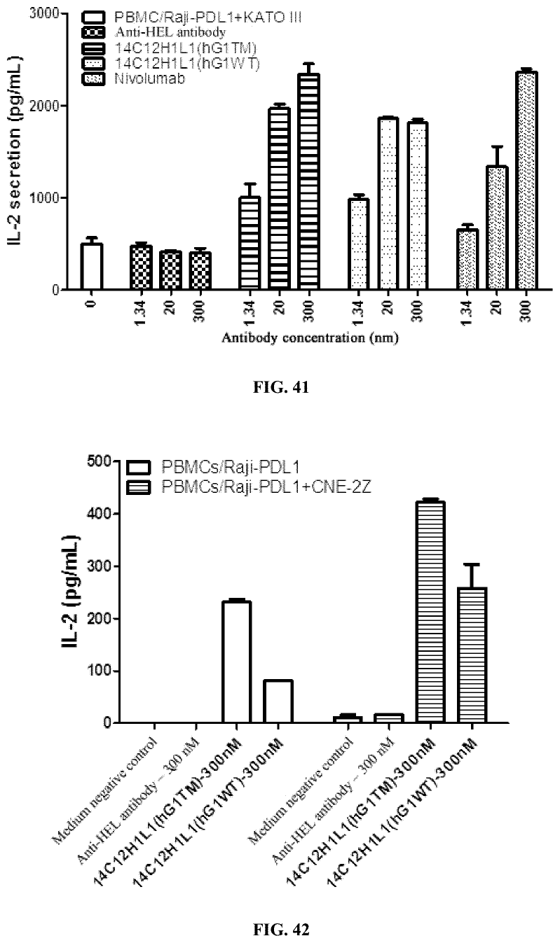

The experimental results are shown in FIG. 41.

The results showed that 14C12H1L1(hG1TM) co-cultured with human gastric cancer KATO III cells exhibited higher pharmacological activity as compared to 14C12H1L1(hG1WT) or nivolumab. 14C12H1L1(hG1TM) can stimulate PBMCs to secrete more IL-2 at the same concentration level, indicating potential for treating gastric cancer.

### Experimental Example 11: Effectively Enhanced Immune Response of Immune Cells to Nasopharyngeal Cancer CNE-2Z Cells by 14C12H1L1(hG1TM)

Raji-PDL1, CNE-2Z cells (purchased from GuangZhou Jennio Biotech Co., Ltd.) and PBMCs were thawed, wherein the PBMCs were stimulated with SEB (0.5 μg/mL) for two days after 2 hours of thawing. On the day of the experiment, Raji-PDL1 cells were treated with MMC (Mito-mycin C with a treatment concentration of 2 μg/mL and a cell treatment density of 200×10⁴ cells/mL) for 1 h. PBMCs were collected, and the treated Raji-PDL1 cells were washed twice with PBS. The PBMCs and the Raji-PDL1 cells were added to the cell plate at 10×10⁴ cells/well, and CNE-2Z cells were added at 3×10⁴ cells/well. The antibodies (with a final concentration of 300 nM and a final volume of 200 μL) were added according to the experimental design, and co-cultured with the cells for 3 days. The culture supernatant was collected and assayed for IL-2. The media in this experiment were all 10% FBS+RPMI 1640.

The results are shown in FIG. 42.

The results showed that 14C12H1L1(hG1TM) co-cultured with human nasopharyngeal cancer CNE-2Z cells exhibited higher pharmacological activity as compared to 14C12H1L1(hG1WT). 14C12H1L1(hG1TM) can stimulate PBMCs to secrete more IL-2 at the same concentration level, indicating potential for treating nasopharyngeal cancer.

### Experimental Example 12: Effectively Enhanced Immune Response of Immune Cells to Mesothelioma NCI-112452 Cells by 14C12H1L1(hG1TM)

Raji-PDL1, NCI-112452 cells (purchased from Chinese Academy of Sciences, Shanghai Institutes for Biological Sciences) and PBMCs were thawed, wherein the PBMCs were stimulated with SEB (0.5 μg/mL) for two days after 2 hours of thawing. On the day of the experiment, Raji-PDL1 cells were treated with MMC (Mito-mycin C with a treatment concentration of 2 μg/mL and a cell treatment density of 200×10⁴ cells/mL) for 1 h. PBMCs were collected, and the treated Raji-PDL1 cells were washed twice with PBS. The PBMCs and the Raji-PDL1 cells were added to the cell plate at 10×10⁴ cells/well, and NCI-112452 cells were added at 3×10⁴ cells/well. The antibodies (with a final concentration of 300 nM and a final volume of 200 μL) were added according to the experimental design, and co-cultured with the cells for 3 days. The culture supernatant was collected and assayed for IL-2. The media in this experiment were all 10% FBS+RPMI 1640.

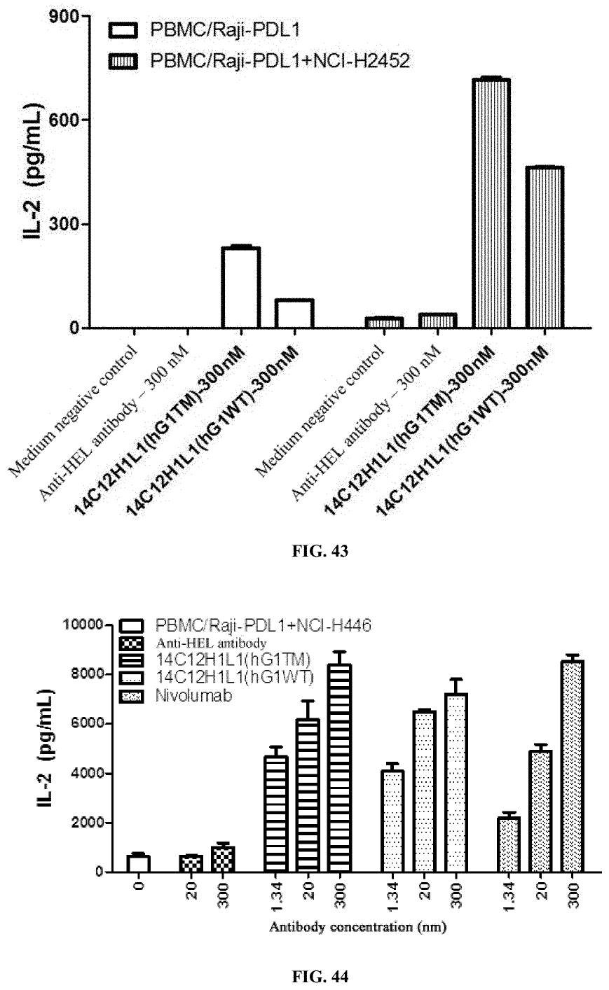

The results are shown in FIG. 43.

The results showed that 14C12H1L1(hG1TM) co-cultured with human mesothelioma NCI-112452 cells exhibited higher pharmacological activity as compared to 14C12H1L1(hG1WT). 14C12H1L1(hG1TM) can stimulate PBMCs to secrete more IL-2 at the same concentration level, indicating potential for treating mesothelioma.

### Experimental Example 12: Effectively Enhanced Immune Response of Immune Cells to Small Cell Lung Cancer NCI-11446 Cells by 14C12H1L1(hG1TM)

PBMCs were isolated from healthy human peripheral blood according to the Ficoll-Paque™ Plus reagent instruction, and the isolated PBMCs were counted and frozen. Raji-PDL1 and NCI-11446 cells (purchased from Chinese Academy of Sciences, Shanghai Institutes for Biological Sciences) were cultured in RPMI 1640+10% FBS complete medium. PBMCs were thawed and activated with 0.5 μg/mL SEB for two days. On the day of the experiment, Raji-PDL1 cells were treated with 2 μg/mL MMC for 1 h. SEB-activated PBMCs and MMC-treated Raji-PDL1 cells were collected, washed twice with PBS, resuspended in RPMI 1640+10% FBS complete medium and counted. Raji-PDL1 and PBMC cells were seeded on 96-well plates at 1×10⁵ cells/well. NCI-11446 cells in logarithmic growth phase were collected and seeded on the 96-well plate at 8×10⁴ cells/well. The diluted antibody was added according to the study design. The mixture was mixed evenly and incubated in a 5% CO₂ incubator at 37° C. for 3 days. After 3 days, the cell culture supernatant was collected and tested for IL-2 according to ELISA KIT instruction. The media in this experiment were all 10% FBS+RPMI 1640.

The results are shown in FIG. 44.

The results showed that 14C12H1L1(hG1TM) co-cultured with human small cell lung cancer NCI-H446 cells exhibited equivalent or higher pharmacological activity as compared to 14C12H1L1(hG1WT) and nivolumab on the basis of effectively eliminated ADCC, CDC and ADCP activities. 14C12H1L1(hG1TM) can stimulate PBMCs to secrete equivalent or more IL-2 at the same concentration level, indicating potential for treating small cell lung cancer.

### Experimental Example 13: Effectively Enhanced Immune Response of Immune Cells to Human Nasopharyngeal Cancer CNE-2Z Cells by 14C12H1L1(hG1TM) in Combination with Anlotinib Hydrochloride

PBMCs were isolated from healthy human peripheral blood according to the Ficoll-Paque™ Plus reagent instruction, and the isolated PBMCs were counted and frozen. Raji-PDL1 and CNE-2Z cells (purchased from GuangZhou Jennio Biotech Co., Ltd.) were cultured in RPMI 1640+10% FBS complete medium. PBMCs were thawed and activated with 0.5 μg/mL SEB for two days. On the day of the experiment, Raji-PDL1 cells were treated with 2 μg/mL MMC for 1 h. SEB-activated PBMCs and MMC-treated Raji-PDL1 cells were collected, washed twice with PBS, resuspended in RPMI 1640+10% FBS complete medium and counted. Raji-PDL1 and PBMC cells were seeded on 96-well plates at 1×10⁵ cells/well. CNE-2Z cells in logarithmic growth phase were collected and seeded on the 96-well plate at 3×10⁴ cells/well. The diluted antibodies and anlotinib were added according to the study design. The mixture was mixed evenly and incubated in a 5% CO₂ incubator at 37° C. for 3 days. After 3 days, the cell culture supernatant was collected and tested for IL-2 according to ELISA KIT instruction. The media in this experiment were all 10% FBS+RPMI 1640.

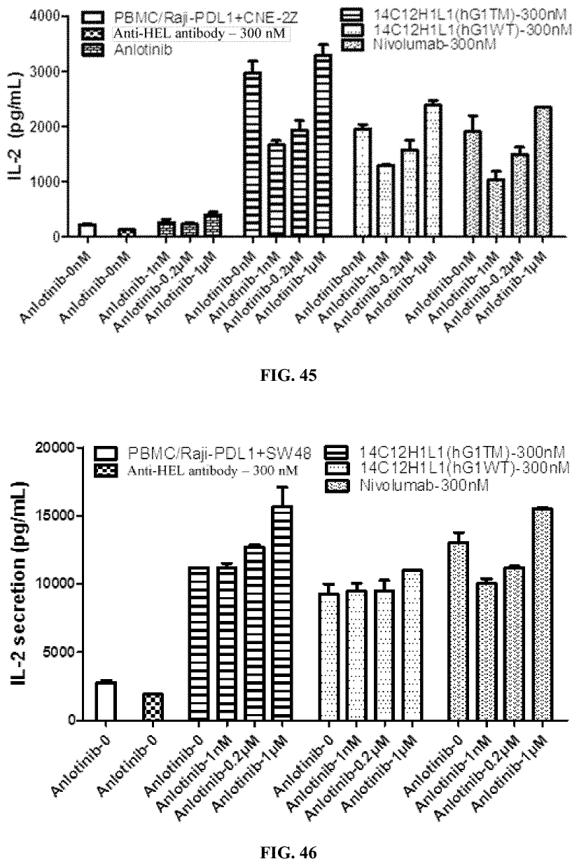

The results are shown in FIG. 45. As compared to the anti-HEL antibody and the anlotinib monotherapies, 14C12H1L1(hG1TM), 14C12H1L1(hG1WT) and nivolumab significantly enhanced the immune response of immune cells to human nasopharyngeal cancer CNE-2Z cells characterized by significantly increased IL-2 secretion level. 14C12H1L1(hG1TM) has superior pharmacological activity than those of 14C12H1L1(hG1WT) and nivolumab. Moreover, the pharmacological activity of 14C12H1L1(hG1TM) in combination with anlotinib in stimulating immune cell activation was superior to those of 14C12H1L1(hG1TM) monotherapy, 14C12H1L1(hG1WT) monotherapy, and nivolumab monotherapy and was also superior to those of 14C12H1L1(hG1WT) in combination with anlotinib and nivolumab in combination with anlotinib.

The above results indicated that 14C12H1L1(hG1TM) in combination with anlotinib has potential for treating human nasopharyngeal cancer.

### Experimental Example 14: Significantly Enhanced Immune Response of Immune Cells to MSI-H/dMMR Tumor SW48 Cells by 14C12H1L1(hG1TM) in Combination with Anlotinib

SW48 is a human colorectal cancer cell line and is identified with MSI-H/dMMR phenotype (Branch P et al., (1995), Cancer Res, 55(11): 2304-2309). It was used for detecting the enhanced immune cell response to tumor of MSI-H/dMMR phenotype by 14C12H1L1(hG1TM).

PBMCs were isolated from healthy human peripheral blood according to the Ficoll-Paque™ Plus reagent instruction, and the isolated PBMCs were counted and frozen. Raji-PDL1 cells were cultured in RPMI 1640+10% FBS complete medium, and SW48 cells (purchased from GuangZhou Jennio Biotech Co., Ltd.) were cultured in DMEM+10% FBS complete medium. PBMCs were thawed and activated with 0.5 μg/mL SEB for two days. On the day of the experiment, Raji-PDL1 cells were treated with 2 μg/mL MMC for 1 h. SEB-activated PBMCs and MMC-treated Raji-PDL1 cells were collected, washed twice with PBS, resuspended in RPMI 1640+10% FBS complete medium and counted. Raji-PDL1 and PBMC cells were seeded on 96-well plates at 1×10⁵ cells/well. SW48 cells in logarithmic growth phase were collected and seeded on the 96-well plate at 2×10⁵ cells/well. The diluted antibody and anlotinib was added according to the study design. The mixture was mixed evenly and incubated in a 5% CO₂ incubator at 37° C. for 3 days. After 3 days, the cell culture supernatant was collected and tested for IL-2 according to ELISA KIT instruction. The media in this experiment were all 10% FBS+RPMI 1640.

The results are shown in FIG. 46.

The results showed that 14C12H1L1(hG1TM), 14C12H1L1(hG1WT) and nivolumab significantly enhanced the immune response of immune cells to human colorectal cancer cells SW48 cells of MSI-H/dMMR phenotype characterized by significantly increased secretion level of IL-2 as compared to anti-HEL antibody. 14C12H1L1(hG1TM) has superior pharmacological activity than that of 14C12H1L1(hG1WT).

Moreover, the pharmacological activity of 14C12H1L1(hG1TM) in combination with anlotinib in stimulating immune cell activation was superior to those of 14C12H1L1(hG1WT) monotherapy, 14C12H1L1(hG1TM) monotherapy, and nivolumab monotherapy and was also superior to those of 14C12H1L1(hG1WT) in combination with anlotinib and nivolumab in combination with anlotinib.

The above results showed that 14C12H1L1(hG1TM) in combination with anlotinib has potential for treating solid tumor of MSI-H/dMMR phenotype, particularly colon cancer and/or rectal cancer of MSI-H/dMMR phenotype.

### Experimental Example 15: Significantly Enhanced Immune Response of Immune Cells to Human Colorectal Cancer SW837 Cells of MSI-H/dMMR Phenotype by 14C12H1L1(hG1TM)

SW837 is a human colorectal cancer cell line of non-MSI-H/dMMR (i.e., MSS) phenotype (Guo J et al., Cancer Res., 2011; 71(8):2978-2987), and was used for detecting the enhanced immune cell response to tumor of non-MSI-H/dMMR (i.e., MSS) phenotype by 14C12H1L1(hG1TM) in this example.

PBMCs were isolated from healthy human peripheral blood according to the Ficoll-Paque™ Plus reagent instruction, and the isolated PBMCs were counted and frozen. Raji-PDL1 cells were cultured in RPMI 1640+10% FBS complete medium, and SW837 cells (purchased from Shanghai Honsun Biological Technology Co., Ltd) were cultured in 10% FBS+Leibovitz's L-15 complete medium (purchased from Gibco). PBMCs were thawed and activated with 0.5 μg/mL SEB for two days. On the day of the experiment, Raji-PDL1 cells were treated with 2 μg/mL MMC for 1 h. SEB-activated PBMCs and MMC-treated Raji-PDL1 cells were collected, washed twice with PBS, resuspended in RPMI 1640+10% FBS complete medium and counted. Raji-PDL1 and PBMC cells were seeded on 96-well plates at 1×10⁵ cells/well. SW837 cells in logarithmic growth phase were collected and seeded on the 96-well plate at 5×10⁴ cells/well. The diluted antibody was added according to the study design. The mixture was mixed evenly and incubated in a 5% CO₂ incubator at 37° C. for 3 days. After 3 days, the cell culture supernatant was collected and tested for IL-2 according to ELISA KIT instruction.

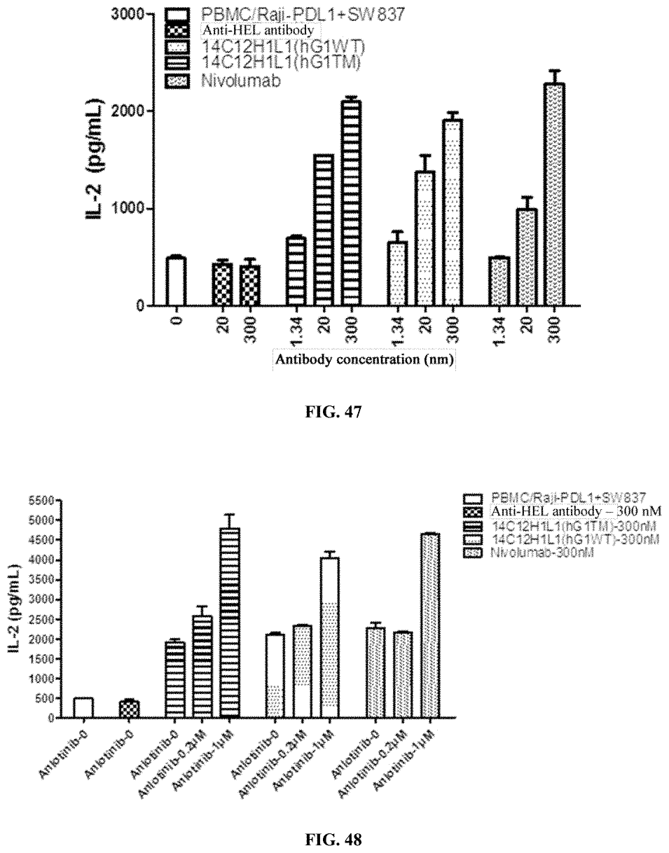

The results are shown in FIG. 47.

The results showed that 14C12H1L1(hG1TM), 14C12H1L1(hG1WT) and nivolumab significantly enhanced the immune response of immune cells to human colorectal cancer cells SW837 cells of non-MSI-H/dMMR phenotype. The pharmacological activity of 14C12H1L1(hG1TM) in the medium and high dose groups was superior to that of 14C12H1L1(hG1WT), characterized by significantly increased secretion level of IL-2. The above results showed that 14C12H1L1(hG1TM) had better or equivalent pharmacological activity relative to 14C12H1L1(hG1WT) and nivolumab on the basis of effectively eliminating ADCC, CDC or ADCP effects, indicating the potential for treating solid tumor of non-MSI-H/dMMR (i.e., MSS) phenotype, particularly colon cancer and/or rectal cancer of non-MSI-H/dMMR phenotype.

### Experimental Example 16: Significantly Enhanced Immune Response of Immune Cells to Human Colorectal Cancer SW837 Cells of MSI-H/dMMR Phenotype by 14C12H1L1(hG1TM) in Combination with Anlotinib

PBMCs were isolated from healthy human peripheral blood according to the Ficoll-Paque™ Plus reagent instruction, and the isolated PBMCs were counted and frozen. Raji-PDL1 cells were cultured in RPMI 1640+10% FBS complete medium and SW837 cells were cultured in Leibovitz's L-15+10% FBS complete medium. PBMCs were thawed and activated with 0.5 μg/mL SEB for two days. On the day of the experiment, Raji-PDL1 cells were treated with 2 μg/mL MMC for 1 h. SEB-activated PBMCs and MMC-treated Raji-PDL1 cells were collected, washed twice with PBS, resuspended in RPMI 1640+10% FBS complete medium and counted. Raji-PDL1 and PBMC cells were seeded on 96-well plates at 1×10⁵ cells/well. SW837 cells in logarithmic growth phase were collected and seeded on the 96-well plate at 5×10⁴ cells/well. The diluted antibody was added according to the study design. The mixture was mixed evenly and incubated in a 5% CO₂ incubator at 37° C. for 3 days. After 3 days, the cell culture supernatant was collected and tested for IL-2 according to ELISA KIT instruction.

The results are shown in FIG. 48.

The results show that as compared to 14C12H1L1(hG1WT) in combination with anlotinib and nivolumab in combination with anlotinib, 14C12H1L1(hG1TM) in combination with anlotinib significantly enhanced the immune response of immune cells to human colorectal cancer SW837 cells of the non-MSI-H/dMMR phenotype characterized by significantly increased IL-2 secretion level, indicating a superior therapeutic effect on solid tumors of non-MSI-H/dMMR phenotype, particularly colon cancer and/or rectal cancer of non-MSI-H/dMMR phenotype.

Although specific embodiments of the present invention have been described in detail, those skilled in the art will understand. Various modifications and substitutions can be made to those details according to all the teachings that have been disclosed, and these changes are all within the protection scope of the present invention. The full scope of the present invention is given by the appended claims and any equivalent thereof.

1. An antibody, wherein
a heavy chain variable region of the antibody comprises HCDR1-HCDR3 with amino acid sequences set forth in SEQ ID NOs: 19-21, respectively, and a light chain variable region of the antibody comprises LCDR1-LCDR3 with amino acid sequences set forth in SEQ ID NOs: 22-24, respectively;
the antibody is of human IgG1 subtype;
wherein, according to the EU numbering system, a heavy chain constant region of the antibody comprises mutations at any 2 or 3 of positions 234,235 and 237, and an affinity constant of the antibody to FcγRIIIa and/or C1q is reduced after the mutation as compared to that before the mutation; preferably, the affinity constant is measured by a Fortebio Octet system.

2. The antibody according to claim 1, wherein according to the EU numbering system, the heavy chain constant region of the antibody comprises the following mutations:
L234A and L235A;
L234A and G237A;
L235A and G237A;
or
L234A, L235A and G237A.

3. An antibody, wherein
a heavy chain variable region of the antibody comprises HCDR1-HCDR3 with amino acid sequences set forth in SEQ ID NOs: 19-21, respectively, and a light chain variable region of the antibody comprises LCDR1-LCDR3 with amino acid sequences set forth in SEQ ID NOs: 22-24, respectively;
the antibody is of human IgG1 subtype;
wherein according to the EU numbering system, a heavy chain constant region of the antibody comprises the following mutations:
L234A and L235A;
L234A and G237A;
L235A and G237A;
or
L234A, L235A and G237A.

4. The antibody according to any of claims 1-3, wherein, according to the EU numbering system, the heavy chain constant region of the antibody further comprises one or more mutations selected from:
N297A, D265A, D270A, P238D, L328E, E233D, 11268D, P271G, A330R, C226S, C229S, E233P, P331S, S267E, L328F, A330L, M252Y, S254T, T256E, N297Q, P238S, P238A, A327Q, A327G, P329A, K322A, T394D, G236R, G236A, L328R, A330S, P331S, H268A, E318A and K320A.

5. The antibody according to any of claims 1-4, wherein
the heavy chain variable region of the antibody comprises an amino acid sequence selected from SEQ ID NO: 2 and SEQ ID NO: 6; and
the light chain variable region of the antibody comprises an amino acid sequence selected from SEQ ID NO: 4 and SEQ ID NO: 8.

6. The antibody according to any of claims 1-4, wherein
the heavy chain variable region of the antibody comprises an amino acid sequence set forth in SEQ ID NO: 2, and the light chain variable region of the antibody comprises an amino acid sequence set forth in SEQ ID NO: 4;
the heavy chain variable region of the antibody comprises an amino acid sequence set forth in SEQ ID NO: 2, and the light chain variable region of the antibody comprises an amino acid sequence set forth in SEQ ID NO: 8;
the heavy chain variable region of the antibody comprises an amino acid sequence set forth in SEQ ID NO: 6, and the light chain variable region of the antibody comprises an amino acid sequence set forth in SEQ ID NO: 4;
or
the heavy chain variable region of the antibody comprises an amino acid sequence set forth in SEQ ID NO: 6, and the light chain variable region of the antibody comprises an amino acid sequence set forth in SEQ ID NO: 8.

7. The antibody according to any of claims 1-6, wherein
the heavy chain is set forth in SEQ ID NO: 16, and the light chain is set forth in SEQ ID NO: 12; or
the heavy chain is set forth in SEQ ID NO: 18, and the light chain is set forth in SEQ ID NO: 12.

8. The antibody according to any of claims 1-7, wherein the antibody binds to FcγRIIIa_F158, FcγRI, FcγRIIa_H131, FcγRIIIa_V158 and/or FcγRIIb with an affinity constant greater than about 10⁻⁷ M, for example, greater than about 10⁻⁶ M, 10⁻⁵ M, 10⁻⁴ M, or 10⁻³ M or greater; preferably, the affinity constant is measured by a Fortebio Octet system;
preferably, the antibody has no binding signal or a binding signal of less than 0.1 nm to FcγRIIIa_F158, FcγRI, FcγRIIa_H131, FcγRIIIay158 and/or FcγRIIb; preferably, the binding signal refers to a response measured by a Fortebio Octet system.

9. The antibody according to any of claims 1-8, wherein the antibody binds to C1q with an affinity constant greater than about 10⁻⁹ M, for example, greater than about 10⁻⁸ M, 10⁻⁷ M, 10⁻⁶ M, or 10⁻⁵ M or greater; preferably, the affinity constant is measured by a Fortebio Octet system;
preferably, the antibody has no binding signal or a binding signal of less than 0.1 nm to C1q;
preferably, the binding signal refers to a response measured by a Fortebio Octet system.

10. An isolated nucleic acid molecule, encoding the antibody according to any of claims 1-9.

11. A vector, comprising the isolated nucleic acid molecule according to claim 10.

12. A host cell, comprising the isolated nucleic acid molecule according to claim 10 or the vector according to claim 11.

13. A conjugate, comprising the antibody according to any of claims 1-9 and a conjugated moiety, wherein the conjugated moiety is a detectable label; preferably, the conjugated moiety is a radioisotope, a fluorescent substance, a luminescent substance, a colored substance, or an enzyme.

14. A kit, comprising the antibody according to any of claims 1-9 or the conjugate according to claim 13;
wherein preferably, the kit further comprises a second antibody specifically recognizing the antibody; optionally, the second antibody further comprises a detectable label, for example, a radioisotope, a fluorescent substance, a luminescent substance, a colored substance, or an enzyme.

15. Use of the antibody according to any of claims 1-9 or the conjugate according to claim 13 in preparing a kit for detecting the presence or level of PD-1 in a sample.

16. A pharmaceutical composition, comprising the antibody according to any of claims 1-9 or the conjugate according to claim 13, wherein, optionally, the pharmaceutical composition further comprises a pharmaceutically acceptable carrier and/or excipient.

17. The pharmaceutical composition according to claim 16, further comprising one or more anti-tumor chemotherapeutics;
preferably, the anti-tumor chemotherapeutic is a tyrosine kinase inhibitor; more preferably, the anti-tumor chemotherapeutic is anlotinib or a pharmaceutically acceptable salt thereof (e.g., hydrochloride salt), or lenvatinib or a pharmaceutically acceptable salt thereof (e.g., mesylate salt).

18. The pharmaceutical composition according to claim 16 or 17, wherein the unit dose of the pharmaceutical composition is 100-1000 mg, 200-800 mg, 200-500 mg, 300-600 mg, 400-500 mg, or 450 mg, based on the mass of the antibody.

19. A therapeutic combination, comprising the antibody according to any of claims 1-9, and at least one (e.g., 1, 2 or 3) anti-tumor chemotherapeutic.

20. The therapeutic combination according to claim 19, wherein the anti-tumor chemotherapeutic is a tyrosine kinase inhibitor; preferably, the anti-tumor chemotherapeutic is anlotinib or a pharmaceutically acceptable salt thereof (e.g., hydrochloride salt), or lenvatinib or a pharmaceutically acceptable salt thereof (e.g., mesylate salt).

21. The therapeutic combination according to claim 19 or 20, wherein the unit dose of the antibody is 100-1000 mg, 200-800 mg, 200-500 mg, 300-600 mg, 400-500 mg, or 450 mg.

22. The therapeutic combination according to claim 19 or 20, wherein the unit dose of the anti-tumor chemotherapeutic is 0.1-100 mg, 0.5-50 mg, 1-20 mg, 2-15 mg, 4-12 mg, or 8-12 mg.

23. The therapeutic combination according to any of claims 19-22, wherein
the therapeutic combination is a fixed combination, e.g., in the form of a solid pharmaceutical composition or a liquid pharmaceutical composition; or
the therapeutic combination is a non-fixed combination, e.g., the anti-PD-1 antibody and the anti-tumor chemotherapeutic in the non-fixed combination are each in the form of a pharmaceutical composition.

24. A kit product, comprising: the pharmaceutical composition according to any of claims 16-18 or the therapeutic combination according to any of claims 19-23, and a package insert.

25. Use of the antibody according to any of claims 1-9, the conjugate according to claim 13, the pharmaceutical composition according to any of claims 16-18 or the therapeutic combination according to any of claims 19-23 in preparing a medicament for treating and/or preventing a tumor or anemia, or in preparing a medicament for diagnosing a tumor or anemia, wherein preferably the tumor is selected from one or more of melanoma, renal cancer, prostate cancer, bladder cancer, colon cancer, rectal cancer, gastric cancer, liver cancer, lung cancer, ovarian cancer, leukemia, nasopharyngeal cancer and endometrial cancer;
preferably, the lung cancer is selected from one or more of non-small cell lung cancer, small cell lung cancer and squamous cell lung cancer;
preferably, the gastric cancer is gastric adenocarcinoma or gastroesophageal junction adenocarcinoma;
preferably, the tumor is a solid tumor of MSI-H/dMMR phenotype; preferably, the tumor is selected from one or more of the following tumors of MSI-H/dMMR phenotype:
colon cancer, rectal cancer, endometrial cancer, gastric cancer, mesothelioma, sarcoma, adrenocortical carcinoma, malignant melanoma and ovarian germ cell neoplasm.

26. Use of the antibody according to any of claims 1-9, the conjugate according to claim 13, the pharmaceutical composition according to any of claims 16-18 or the therapeutic combination according to any of claims 19-23 in preparing:
a medicament for blocking the binding of PD-1 to PD-L1,
a medicament for down-regulating the activity or level of PD-1,
a medicament for relieving the immunosuppression of PD-1 in an organism, or
a medicament for elevating IFN-γ and/or IL-2 expression in T lymphocytes.

27. The antibody according to any of claims 1-9, the conjugate according to claim 13, the pharmaceutical composition according to any of claims 16-18 or the therapeutic combination according to any of claims 19-23 for use in treating and/or preventing a tumor or anemia, or for use in diagnosing a tumor or anemia, wherein preferably the tumor is selected from one or more of melanoma, renal cancer, prostate cancer, bladder cancer, colon cancer, rectal cancer, gastric cancer, liver cancer, lung cancer, ovarian cancer, leukemia, nasopharyngeal cancer and endometrial cancer;
preferably, the lung cancer is selected from one or more of non-small cell lung cancer, small cell lung cancer and squamous cell lung cancer;
preferably, the gastric cancer is gastric adenocarcinoma or gastroesophageal junction adenocarcinoma;
preferably, the tumor is a solid tumor of MSI-H/dMMR phenotype; preferably, the tumor is selected from one or more of the following tumors of MSI-H/dMMR phenotype:
colon cancer, rectal cancer, endometrial cancer, gastric cancer, mesothelioma, sarcoma, adrenocortical carcinoma, malignant melanoma and ovarian germ cell neoplasm.

28. A method for treating and/or preventing a tumor or anemia, or a method of diagnosing a tumor or anemia, comprising: administering to a subject in need an effective amount of the antibody according to any of claims 1-9, the conjugate according to claim 13, the pharmaceutical composition according to any of claims 16-18 or the therapeutic combination according to any of claims 19-23, wherein preferably the tumor is selected from one or more of melanoma, renal cancer, prostate cancer, bladder cancer, colon cancer, rectal cancer, gastric cancer, liver cancer, lung cancer, ovarian cancer, leukemia, nasopharyngeal cancer and endometrial cancer;
preferably, the lung cancer is selected from one or more of non-small cell lung cancer, small cell lung cancer and squamous cell lung cancer;
preferably, the gastric cancer is gastric adenocarcinoma or gastroesophageal junction adenocarcinoma;
preferably, the tumor is a solid tumor of MSI-H/dMMR phenotype; preferably, the tumor is selected from one or more of the following tumors of MSI-H/dMMR phenotype:
colon cancer, rectal cancer, endometrial cancer, gastric cancer, mesothelioma, sarcoma, adrenocortical carcinoma, malignant melanoma and ovarian germ cell neoplasm.

29. The method according to claim 28, wherein the effective amount of the antibody is administered to the subject in need before or after surgical treatment and/or before or after radiotherapy.

30. The method according to claim 28 or 29, wherein the unit dose of the antibody is 0.1-100 mg per kg body weight, preferably 1-10 mg per kg body weight; alternatively, the unit dose of the antibody is 10-1000 mg, preferably 50-500 mg in each subject;
preferably, the dose is given once every 3 days, 4 days, 5 days, 6 days, 10 days, 1 week, 2 weeks or 3 weeks;
preferably, the route of administration is intravenous drip infusion or intravenous injection.

31. The method according to any of claims 28-30, wherein the administration of the antibody is performed in cycles of 2 or 3 weeks, and preferably, the antibody is administered intravenously on the first day of each cycle; preferably, the antibody is administered once every two or three weeks.


SEQUENCE LISTING (24 sequences)

SEQ ID NO: 1 - DNA, 354 bp, Artificial
  Nucleic acid sequence of 14C12 heavy chain variable region
gaggtcaaactggtggagagcggcggcgggctggtgaagcccggcgggtcactgaaactgagctgcgccgcttccggcttcgcctttagctcctacgacatgtcatgggtgaggcagacccctgagaagcgcctggaatgggtcgctactatcagcggaggcgggcgatacacctactatcctgactctgtcaaagggagattcacaattagtcgggataacgccagaaatactctgtatctgcagatgtctagtctgcggtccgaggatacagctctgtactattgtgcaaaccggtacggcgaagcatggtttgcctattggggacagggcaccctggtgacagtctctgcc

SEQ ID NO: 2 - protein, 118 aa, Artificial
  The amino acid sequence of 14C12 heavy chain variable region
EVKLVESGGGLVKPGGSLKLSCAASGFAFSSYDMSWVRQTPEKRLEWVATISGGGRYTYYPDSVKGRFTISRDNARNTLYLQMSSLRSEDTALYYCANRYGEAWFAYWGQGTLVTVSA

SEQ ID NO: 3 - DNA, 321 bp, Artificial
  Nucleic acid sequence of 14C12 light chain variable region
gacattaagatgacacagtccccttcctcaatgtacgctagcctgggcgagcgagtgaccttcacatgcaaagcatcccaggacatcaacacatacctgtcttggtttcagcagaagccaggcaaaagccccaagaccctgatctaccgggccaatagactggtggacggggtccccagcagattctccggatctggcagtgggcaggattactccctgaccatcagctccctggagtatgaagacatgggcatctactattgcctgcagtatgatgagttccctctgacctttggagcaggcacaaaactggaactgaag

SEQ ID NO: 4 - protein, 107 aa, Artificial
  The amino acid sequence of 14C12 light chain variable region
DIKMTQSPSSMYASLGERVTFTCKASQDINTYLSWFQQKPGKSPKTLIYRANRLVDGVPSRFSGSGSGQDYSLTISSLEYEDMGIYYCLQYDEFPLTFGAGTKLELK

SEQ ID NO: 5 - DNA, 354 bp, Artificial
  Nucleic acid sequence of 14C12H1L1(hG1WT) heavy chain variable region
gaagtgcagctggtcgagtctgggggagggctggtgcagcccggcgggtcactgcgactgagctgcgcagcttccggattcgcctttagctcctacgacatgtcctgggtgcgacaggcaccaggaaagggactggattgggtcgctactatctcaggaggcgggagatacacctactatcctgacagcgtcaagggccggttcacaatctctagagataacagtaagaacaatctgtatctgcagatgaacagcctgagggctgaggacaccgcactgtactattgtgccaaccgctacggggaagcatggtttgcctattgggggcagggaaccctggtgacagtctctagt

SEQ ID NO: 6 - protein, 118 aa, Artificial
  The amino acid sequence of 14C12H1L1(hG1WT) heavy chain variable region
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSS

SEQ ID NO: 7 - DNA, 321 bp, Artificial
  Nucleic acid sequence of 14C12H1L1(hG1WT) light chain variable region
gacattcagatgactcagagcccctcctccatgtccgcctctgtgggcgacagggtcaccttcacatgccgcgctagtcaggatatcaacacctacctgagctggtttcagcagaagccagggaaaagccccaagacactgatctaccgggctaatagactggtgtctggagtcccaagtcggttcagtggctcagggagcggacaggactacactctgaccatcagctccctgcagcctgaggacatggcaacctactattgcctgcagtatgatgagttcccactgacctttggcgccgggacaaaactggagctgaag

SEQ ID NO: 8 - protein, 107 aa, Artificial
  The amino acid sequence of 14C12H1L1(hG1WT) light chain variable region
DIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELK

SEQ ID NO: 9 - DNA, 1344 bp, Artificial
  Nucleic acid sequence of 14C12H1L1 (hG1WT) heavy chain
gaagtgcagctggtcgagtctgggggagggctggtgcagcccggcgggtcactgcgactgagctgcgcagcttccggattcgcctttagctcctacgacatgtcctgggtgcgacaggcaccaggaaagggactggattgggtcgctactatctcaggaggcgggagatacacctactatcctgacagcgtcaagggccggttcacaatctctagagataacagtaagaacaatctgtatctgcagatgaacagcctgagggctgaggacaccgcactgtactattgtgccaaccgctacggggaagcatggtttgcctattgggggcagggaaccctggtgacagtctctagtgccagcaccaaaggacctagcgtgtttcctctcgccccctcctccaaaagcaccagcggaggaaccgctgctctcggatgtctggtgaaggactacttccctgaacccgtcaccgtgagctggaatagcggcgctctgacaagcggagtccatacattccctgctgtgctgcaaagcagcggactctattccctgtccagcgtcgtcacagtgcccagcagcagcctgggcacccagacctacatctgtaacgtcaaccacaagccctccaacaccaaggtggacaagaaagtggagcccaaatcctgcgacaagacacacacctgtcccccctgtcctgctcccgaactcctcggaggccctagcgtcttcctctttcctcccaaacccaaggacaccctcatgatcagcagaacccctgaagtcacctgtgtcgtcgtggatgtcagccatgaggaccccgaggtgaaattcaactggtatgtcgatggcgtcgaggtgcacaacgccaaaaccaagcccagggaggaacagtacaactccacctacagggtggtgtccgtgctgacagtcctccaccaggactggctgaacggcaaggagtacaagtgcaaggtgtccaacaaggctctccctgcccccattgagaagaccatcagcaaggccaaaggccaacccagggagccccaggtctatacactgcctccctccagggacgaactcaccaagaaccaggtgtccctgacctgcctggtcaagggcttttatcccagcgacatcgccgtcgagtgggagtccaacggacagcccgagaataactacaagaccacccctcctgtcctcgactccgacggctccttcttcctgtacagcaagctgaccgtggacaaaagcaggtggcagcagggaaacgtgttctcctgcagcgtgatgcacgaagccctccacaaccactacacccagaaaagcctgtccctgagccccggcaaa

SEQ ID NO: 10 - protein, 448 aa, Artificial
  Amino acid sequence of 14C12H1L1 (hG1WT) heavy chain
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 642 bp, Artificial
  Nucleic acid sequence of 14C12H1L1 (hG1WT) light chain
gacattcagatgactcagagcccctcctccatgtccgcctctgtgggcgacagggtcaccttcacatgccgcgctagtcaggatatcaacacctacctgagctggtttcagcagaagccagggaaaagccccaagacactgatctaccgggctaatagactggtgtctggagtcccaagtcggttcagtggctcagggagcggacaggactacactctgaccatcagctccctgcagcctgaggacatggcaacctactattgcctgcagtatgatgagttcccactgacctttggcgccgggacaaaactggagctgaagcgaactgtggccgctccctccgtcttcatttttcccccttctgacgaacagctgaaatcaggcacagccagcgtggtctgtctgctgaacaatttctaccctagagaggcaaaagtgcagtggaaggtcgataacgccctgcagtccggcaacagccaggagagtgtgactgaacaggactcaaaagatagcacctattccctgtctagtacactgactctgtccaaggctgattacgagaagcacaaagtgtatgcatgcgaagtgacacatcagggactgtcaagccccgtgactaagtcttttaaccggggcgaatgt

SEQ ID NO: 12 - protein, 214 aa, Artificial
  Amino acid sequence of the light chain of 14C12H1L1 (hG1WT)
DIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1335 bp, Artificial
  Nucleic acid sequence of 14C12H1L1(hG4) heavy chain
gaagtgcagctggtcgagtctgggggagggctggtgcagcccggcgggtcactgcgactgagctgcgcagcttccggattcgcctttagctcctacgacatgtcctgggtgcgacaggcaccaggaaagggactggattgggtcgctactatctcaggaggcgggagatacacctactatcctgacagcgtcaagggccggttcacaatctctagagataacagtaagaacaatctgtatctgcagatgaacagcctgagggctgaggacaccgcactgtactattgtgccaaccgctacggggaagcatggtttgcctattgggggcagggaaccctggtgacagtctctagtgccagcaccaaagggccctcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 14 - protein, 445 aa, Artificial
  Amino acid sequence of 14C12H1L1(hG4) heavy chain
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - DNA, 1344 bp, Artificial
  Nucleic acid sequence of 14C12H1L1(hG1TM) heavy chain
gaagtgcagctggtcgagtctgggggagggctggtgcagcccggcgggtcactgcgactgagctgcgcagcttccggattcgcctttagctcctacgacatgtcctgggtgcgacaggcaccaggaaagggactggattgggtcgctactatctcaggaggcgggagatacacctactatcctgacagcgtcaagggccggttcacaatctctagagataacagtaagaacaatctgtatctgcagatgaacagcctgagggctgaggacaccgcactgtactattgtgccaaccgctacggggaagcatggtttgcctattgggggcagggaaccctggtgacagtctctagtgccagcaccaaagggcccagcgtgtttcctctcgccccctcctccaaaagcaccagcggaggaaccgctgctctcggatgtctggtgaaggactacttccctgaacccgtcaccgtgagctggaatagcggcgctctgacaagcggagtccatacattccctgctgtgctgcaaagcagcggactctattccctgtccagcgtcgtcacagtgcccagcagcagcctgggcacccagacctacatctgtaacgtcaaccacaagccctccaacaccaaggtggacaagaaagtggagcccaaatcctgcgacaagacacacacctgtcccccctgtcctgctcccgaagctgctggagcccctagcgtcttcctctttcctcccaaacccaaggacaccctcatgatcagcagaacccctgaagtcacctgtgtcgtcgtggatgtcagccatgaggaccccgaggtgaaattcaactggtatgtcgatggcgtcgaggtgcacaacgccaaaaccaagcccagggaggaacagtacaactccacctacagggtggtgtccgtgctgacagtcctccaccaggactggctgaacggcaaggagtacaagtgcaaggtgtccaacaaggctctccctgcccccattgagaagaccatcagcaaggccaaaggccaacccagggagccccaggtctatacactgcctccctccagggacgaactcaccaagaaccaggtgtccctgacctgcctggtcaagggcttttatcccagcgacatcgccgtcgagtgggagtccaacggacagcccgagaataactacaagaccacccctcctgtcctcgactccgacggctccttcttcctgtacagcaagctgaccgtggacaaaagcaggtggcagcagggaaacgtgttctcctgcagcgtgatgcacgaagccctccacaaccactacacccagaaaagcctgtccctgagccccggcaaa

SEQ ID NO: 16 - protein, 448 aa, Artificial
  Amino acid sequence of 14C12H1L1(hG1TM) heavy chain
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 1344 bp, Artificial
  Nucleic acid sequence of 14C12H1L1(hG1DM) heavy chain
gaagtgcagctggtcgagtctgggggagggctggtgcagcccggcgggtcactgcgactgagctgcgcagcttccggattcgcctttagctcctacgacatgtcctgggtgcgacaggcaccaggaaagggactggattgggtcgctactatctcaggaggcgggagatacacctactatcctgacagcgtcaagggccggttcacaatctctagagataacagtaagaacaatctgtatctgcagatgaacagcctgagggctgaggacaccgcactgtactattgtgccaaccgctacggggaagcatggtttgcctattgggggcagggaaccctggtgacagtctctagtgctagcaccaaagggcccagcgtgtttcctctcgccccctcctccaaaagcaccagcggaggaaccgctgctctcggatgtctggtgaaggactacttccctgaacccgtcaccgtgagctggaatagcggcgctctgacaagcggagtccatacattccctgctgtgctgcaaagcagcggactctattccctgtccagcgtcgtcacagtgcccagcagcagcctgggcacccagacctacatctgtaacgtcaaccacaagccctccaacaccaaggtggacaagaaagtggagcccaaatcctgcgacaagacacacacctgtcccccctgtcctgctcccgaagctgctggaggccctagcgtcttcctctttcctcccaaacccaaggacaccctcatgatcagcagaacccctgaagtcacctgtgtcgtcgtggatgtcagccatgaggaccccgaggtgaaattcaactggtatgtcgatggcgtcgaggtgcacaacgccaaaaccaagcccagggaggaacagtacaactccacctacagggtggtgtccgtgctgacagtcctccaccaggactggctgaacggcaaggagtacaagtgcaaggtgtccaacaaggctctccctgcccccattgagaagaccatcagcaaggccaaaggccaacccagggagccccaggtctatacactgcctccctccagggacgaactcaccaagaaccaggtgtccctgacctgcctggtcaagggcttttatcccagcgacatcgccgtcgagtgggagtccaacggacagcccgagaataactacaagaccacccctcctgtcctcgactccgacggctccttcttcctgtacagcaagctgaccgtggacaaaagcaggtggcagcagggaaacgtgttctcctgcagcgtgatgcacgaagccctccacaaccactacacccagaaaagcctgtccctgagccccggcaaa

SEQ ID NO: 18 - protein, 448 aa, Artificial
  Amino acid sequence of 14C12H1L1(hG1DM) heavy chain
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 8 aa, Artificial
  HCDR1
GFAFSSYD

SEQ ID NO: 20 - protein, 8 aa, Artificial
  HCDR2
ISGGGRYT

SEQ ID NO: 21 - protein, 11 aa, Artificial
  HCDR3
ANRYGEAWFAY

SEQ ID NO: 22 - protein, 6 aa, Artificial
  LCDR1
QDINTY

SEQ ID NO: 23 - protein, 3 aa, Artificial
  LCDR2
RAN

SEQ ID NO: 24 - protein, 9 aa, Artificial
  LCDR3
LQYDEFPLT